(19) United States
(12) Patent Application Publication
(10) Pub. No.: US 2025/0027165 A1
(43) Pub. Date: Jan. 23, 2025

(54) ASSAY

(71) Applicant: CAMBRIDGE ENTERPRISE LIMITED, Cambridge (GB)

(72) Inventors: Richard GILBERTSON, Cambridge (GB); Eric RAHRMANN, Cambridge (GB); David SHORTHOUSE, Cambridge (GB)

(21) Appl. No.: 18/714,695

(22) PCT Filed: Dec. 2, 2022
(86) PCT No.: PCT/GB2022/053056

§ 371 Date: May 30, 2024

(30) Foreign Application Priority Data
Dec. 3, 2021 (GB) 2117513.8

(51) Int. Cl. C12Q 1/6886; G01N 33/574; G16B 5/00; G16H 50/30

(52) U.S. Cl. CPC C12Q 1/6886; G01N 33/57492; G16B 5/00; G16H 50/30; C12Q 2600/156

(57) ABSTRACT

A method for the detection or prognosis of cancer and/or metastasis is provided. Tumour samples may be used to determine the presence of a mutation within the sodium leak channel (NALCN). A risk score of cancer and/or metastasis can be determined based on if the mutation causes a reduction in the pore size of NALCN. A computational model, a composition, and a kit are also provided.

### FIELD OF INVENTION

The invention relates to methods for the detection or prognosis of cancer and/or metastasis comprising detecting mutations and/or a reduction in activity in sodium leak channel (NALCN).

### BACKGROUND

Most patients with cancer die as a result of metastasis (Dillekås et al, 2019)—the process by which cancer cells spread from the primary tumour to other organs in the body (Ganesh, K. & Massagué, J, 2021). Cancers cells can spread throughout the body via various mechanisms and some of them are able to form new tumours in other parts of the body. Metastatic cancer cells can also remain inactive at a distant site for long periods of time before they begin to grow again, if at all. Blocking metastasis could markedly improve the survival of patients with cancer; but how this process is triggered within the complex cascade of tumourigenesis remains unclear (Massague, J. & Obenauf, A. C., 2016).

Because metastasis is thought to be a wholly abnormal process, restricted to malignant tissues, attention has focused on identifying genetic mutations as drivers of cancer metastasis. Although this research has unmasked that promote metastasis in mouse models and humans, including a variety of ion channels that induce a metastasis-like phenotype by altering the transmembrane voltage to induce changes in gene transcription (House, C. D. et al., 2010, Sheth, M. & Esfandiari, L., 2022, and Wang T. et al, 2020), so far no recurrent metastasis-specific mutations have been identified (Ganesh, K. & Massagué, J, 2021, Massagué, J. & Obenauf, A. C., 2016, and Nguyen, B. et al. 2022).

Other cell functions implicated in the metastatic cascade include ‘stem cell-like’ multipotency and plasticity. Stem cell capacity has been ascribed to metastatic cancer cells because of their ability to reconstitute heterogenous malignant cell populations as metastatic tumors (Ganesh, K. et al 2020, and Laughney, A. M. et al. 2020). Epithelial mesenchymal transition (EMT) (Ganesh, K. & Massagué, J, 2021)—a type of cellular plasticity displayed during normal gastrulation and tissue healing—is also an established feature of the metastatic cascade (Ganesh, K. & Massagué, J, 2021 and Pastushenko, I. et al. 2018). What remains unclear is how cancers ‘hijack’ these normal cell functions to enable metastasis.

As such, there is a need to develop methods to detect metastasis and cancer. In the present application, we identify a single ion channel, NALCN, as a key regulator of epithelial cell trafficking to distant tissues. NALCN is responsible for the background sodium leak conductance that maintains the resting membrane potential. It regulates key functions in excitable tissues, for example, respiration and circadian rhythms (Chua, H. C. et al 2020, Kschonsak, M. et al. 2020, and Lu, B. et al 2007) and gain-of-function mutations in the gene are associated with neurological disorders (Bend, E. G. et al. 2016). However, little is known about the role of NALCN in nonexcitable tissues. The present invention demonstrates that NALCN regulates the release of malignant and normal epithelial cells into the blood, and their trafficking to distant sites where they form metastatic cancers, or apparently normal tissues, respectively. We thereby demonstrate that the metastatic cascade can be triggered and operate independent of tumorigenesis. These observations have profound implications for understanding epithelial cell trafficking in health and disease and identify a novel target for antimetastatic therapies.

### SUMMARY OF THE INVENTION

The present inventors have identified a single ion channel, NALCN, as a key regulator of both malignant and non-malignant cell metastasis, providing important insights to the metastatic process and a novel target for anti-metastatic therapies. Among 10,022 human cancers, NALCN loss-of-function mutations were selectively enriched in advanced gastric and colorectal cancers. Deletion of Nalcn from mice susceptible to developing gastric, intestinal or pancreatic adenocarcinoma did not alter the incidence of these tumours, but markedly increased levels of circulating tumour cells (CTCs) and seeding of peritoneal, kidney, liver and lung metastases. Treatment of these mice with gadolinium-a Nalcn channel blocker—similarly increased CTCs and metastasis. Remarkably, deletion of Nalcn from mice that lacked oncogenic mutations and never developed cancer, caused similar shedding of cells into the peripheral blood at levels equivalent to those seen in tumour-bearing animals. These cells trafficked to distant organs where they formed apparently normal structures, including kidney glomeruli and tubules, rather than tumours. The transcriptomes of these circulating cells in tumour and non-tumour-bearing mice were indistinguishable and closely related to those of human CTCs. Thus, NALCN regulates cell shedding from solid tissues independent of cancer, divorcing this process from tumourigenesis and unmasking NALCN as a key mediator of metastasis.

An aspect of the invention relates to a method for the detection or prognosis of cancer and/or metastasis comprising:
analysing a tumour sample obtained from a subject,
determining the presence of at least one mutation within sodium leak channel (NALCN) in the tumour sample, compared to a reference sample,
determining whether the at least one mutation causes a reduction in the pore size of NALCN,
where the mutation causes a reduction in the pore size of NALCN, using the reduction in pore size to determine a risk score of cancer and/or metastasis.

An aspect of the invention relates to a method for the detection or prognosis of cancer and/or metastasis comprising:
analysing a biological sample obtained from a subject, to assess the activity of sodium leak channel (NALCN),
providing a risk score of cancer and/or metastasis based on the level of activity of NALCN.

An aspect of the invention relates to a method for the detection or prognosis of cancer and/or metastasis comprising:
analysing a biological sample to detect the presence of one or more mutations which correspond to a reduction of function of NALCN
providing a risk score of cancer and/or metastasis based on the presence of one or more mutations which correspond to a reduction of function of NALCN.

An aspect of the invention relates to a method for determining the activity of NALCN comprising:
analysing a biological sample to detect one or more mutations identified in Table 2
wherein the presence of one or more mutation identified in Table 2 indicates reduced activity of NALCN.

An aspect of the invention relates to a kit comprising reagents for the detection of one or more mutations in NALCN identified in Table 2 and optionally instructions for use.

An aspect of the invention relates to a composition comprising reagents for the detection of one or more mutations in NALCN identified in Table 2.

An aspect of the invention relates to a computer-implemented method for determine a risk score of cancer and/or metastasis, the method comprising obtaining data indicating presence of at least one mutation in a sodium leak channel, NALCN, in a tumour sample, inputting the data into a computational model of NALCN that simulates effects of mutations on NALCN, determining, using the computational model, whether the at least one mutation causes a reduction in a pore size of NALCN, and outputting, when the at least one mutation is determined to cause a reduction in pore size of NALCN, a risk score of cancer and/or metastasis.

### FIGURES

FIG. 1. NALCN loss-of-function characterises aggressive intestinal cancer. (a) Volcano plot of differential gene expression between Prom1⁺ cells isolated from mouse stomach epithelium and P1^{KP}-GACs: down-regulated ion channels highlighted. (b) t-Distributed Stochastic Neighbor Embedding plot of 10,022 samples from 32 human cancer types: NALCN-mutant samples and cancer-types enriched for NALCN mutations highlighted (p-value, dN/dS shown). (c) Mutant residues significantly enriched in pore turret and voltage sensing domains of NALCN. (d) Impact of 196 different NALCN mutations on pore radius determined by HOLE analysis. (e) NALCN pore closure caused by mutations in different stages of cancer (*=p<0.05, Mann-Whitney).

FIG. 2. NALCN loss-of-function increases tumour metastasis. (a) Unsupervised hierarchical clustering of 77 primary and metastatic P1^{KP}-GAC, V1^{KP}-IAC, Pdx1^{KP}-PAC tumours as well as four P1; Pten^{Flx/Flx}; Tp53^{Flx/Flx} (P1^{PtP}) primary hepatocellular carcinomas. Heatmap below reports enrichment of the indicated primary tumour transcriptome in each metastatic tumour. (b) Macroscopic images of exemplar ZsGreen⁺ (ZSG) metastatic tumours ([met] outlined). (c) Photomicrographs (left haematoxylin and eosin, right immunohistochemistry/fluorescence) of the metastases in (b). Scale bar 50 μm. (d) Left in each, cumulative total number of metastases per autopsied mouse of the indicated genotype at the indicated time post tamoxifen induction (age for Pdx1^{KP} mice; p=Mann-Whitney for total tumour burden in Nalcn-deleted versus wild-type mice, see also Supplementary Tables 5 and 6). Right in each, organ heat maps of total metastases per mouse of the indicated genotype. Numbers of male:female (M:F) and P3:P60 induced mice are shown. (e) Cumulative metastatic burden and organ metastases heatmap plots of V1KP-IAC in gadolinium or control treated Nalcn^{+/+} mice. (*=p<0.05, **=p<0.005, Mann-Whitney).

FIG. 3. NALCN loss-of-function increases shedding of circulating tumour cells. (a) Scatter plot of CZCs (expressed as % of total peripheral blood cells) identified in peripheral blood of the indicated mice that were, or were not treated with gadolinium (ns=not significant, *=p<0.05, **=p<0.005, Mann-Whitney). (b) Uniform Manifold Approximation and Projection (UMAP) of single cell RNA sequencing profiles of CZCs and mouse peripheral blood mononuclear cells. (c) Heatmap reporting geneset enrichment analysis in the UMAP clusters identified in (b). Test Genesets were derived from 2,086 different tissue and cell types including bulk RNAseq of mouse normal tissues and tumours, huCTC signatures, and normal PBMCs (Methods). (d) Exemplar co-immunofluorescence of CZCs and PBMCs in peripheral blood smears of P₁KP (top) and V1^{KP} (bottom) mice (ZsGreen [ZSG], scale bar=10 μm). (e) Top left, exemplar macroscopic direct green fluorescence imaging of a whole mouse lung showing Pdx1^{KP}-PAC CZC metastases in a recipient immunocompromised mouse. Other images show exemplar haematoxylin and eosin or co-immunofluorescence of metastases of P1^{KP}-GAC or V1^{KP}-IAC CZC metastases in immunocompromised recipient mice (scale bar=10 μm). (f) Organ heat maps of total metastases per mouse identified in recipient mice injected with the indicating CZCs.

FIG. 4. NALCN loss-of-function increases shedding of circulating non-tumour cells. (a) Scatter plot of CZCs (expressed as % of total cells) identified in peripheral blood of the indicated non-tumour bearing mice (***=p<0.0005, Mann-Whitney). (b) Uniform Manifold Approximation and Projection (UMAP) of 201,183 single cell RNA sequencing profiles (SCS) of PBMCs and tumour bearing (t) and non-tumour bearing (nt) CZCs as well as cells derived from the indicated normal and malignant mouse tissues. (c) Exemplar co-immunofluorescence of CZCs and PBMCs in peripheral blood smears of P1^{R}Nalcn^{Flx/Flx} mice (ZsGreen [ZSG], scale bar=10 μm). (d) Organ heat maps of total numbers of CZC cell clusters per mouse identified in organs of recipient mice injected with the indicated P1^{R}Nalcn^{Flx/Flx} CZCs. (e) Exemplar co-immunofluorescence of P1^{R}Nalcn^{Flx/Flx} CZCs (arrows) incorporated into the kidneys of recipient mice (arrows indicated ZSG'⁰ cells, scale bar=50 μm). (f) Confocal laser scanning microscope image of P1^{R}Nalcn^{Flx/Flx} CZCs incorporated into the renal cortex of recipient mice (scale bar=100 μm).

FIG. 5. Nalcn deletion does not impact the incidence, tumor-free survival or growth rates of P1KP, V1KP or Pdx1KP primary tumors. a-c Tumors and representative photomicrographs (H&E from all tumors (left; Supplementary Table 9) and dual immunofluorescence from five independent tumors each (right)) for lineage tracing (ZSG), epithelial (CK7, CK20) and EMT markers (CDH2, CDH1) of P1KP-GAC (a), V1KP-IAC (b) and Pdx1 KP-PAC (c). Scale bar, 50 μm. d-g, Upper: organ heatmaps of tumor incidence in P1 KP at P3 and V1 KP at mice of each Nalcn genotype recombined at P3 (d,e) or P60 (f,g). Lower: survival curves of mice in each cohort. Male to female ration (M:F) is shown. P1KP P3, P=0.6912; P1KP P60, P=0.3897; V1KP P3, P=0.1900; and V1KP P60, P=0.8301. Mantel-Cox test. h, Organ primary tumor heatmaps and survival curves of Pdx1 KP mice (P=0.1095). Mantel-Cox test. Source data for d-h are given in Supplementary Table 9. i, Growth rates of P1KP-GAC (n=38), V1KP-IAC (n=57) and Pdx1KP-PAC (n=28) tumors. Two-tailed Mann-Whitney U-tests revealed no significant difference in growth rates among tumors with different Nalcn genotypes P1KP-GAC: Nalcn+/+(n=11) versus Nalcn+/Flx (n=18; P=0.912), versus NalcnFlx/Flx (n=9; P=0.7103). V1 KP-IAC: Nalcn+/+(n=16) versus Nalcn+/Flx (n=25; P=0.5169), versus NalcnFlx/Flx (n=16; P=0.7309). Pdx1KP-PAC: Nalcn+/+(n=10) versus Nalcn+/Flx (n=13; P=0.7844), versus NalcnFlx/Flx (n=5; P=0.1292). Bar, median. Source data are given in Supplementary Table 10. j, Gene set enrichment analyses of transcriptomes of Nalcn+/Flx and NalcnFlx/Flx P1 KP-GAC, V1 KP-IAC and Pdx1 KP-PAC versus Nalcn+/+ tumors.

FIG. 6. Nalcn deletion does not affect cell proliferation, apoptosis, immune-infiltration, vasculature or ASMA expression in primary tumours in P1^{KP}, V1^{KP} or Pdx1^{KP} mice. (a) HALO-quantification of Nalcn mRNA transcripts per cell detected by RNA-scope analysis in mouse gastric (GAC), intestinal (IAC) and pancreatic (PAC) adenocarcinomas of the indicated Nalcn genotype (bar=median; *p=0.0294; ***p=0.0004; ****=p<0.0001, two-tailed Mann-Whitney test). Data are tumour fields (5-8 images pertumour) from n=5 tumours for each Nalcn genotype of P1^{KP}-GAC, V1^{KP}-IAC and Pdx1^{KP}-PAC mice (total n=45 unique tumours, 289 unique tumour fields). (b) Representative photomicrographs of Nalcn RNA in situ hybridization in GACs (n=15 biologically distinct tumours, 100 tumour fields) of the indicated Nalcn genotype (scale=50 μm). (c) Left in each is HALO-quantification (Data are mean±SD) of immunohistochemically-detected tumour cell expression of MKI67 (proliferation), cleaved Caspase-3 (CC3; apoptosis), CD45 (immune cell infiltration), CD31 (endothelial cells) and alpha-smooth muscle actin ASMA; stroma) in five complete biologically independent tumour fields for each Nalcn genotype of P1^{KP}-GAC, V1^{KP}-IAC and Pdx1^{KP}-PAC mice (total n=45 unique tumours). Two-tailed Mann-Whitney U tests revealed no significant difference in these markers among tumours with different Nalcn genotypes. P-values GAC, IAC, PAC of Nalcn^{+/+} vs Nalcn^{+/Flx}, Nalcn^{+/+} vs Nalcn^{Flx/Flx}, respectively: KI67 (0.4206, 0.4206, 0.4206, 0.5476, 0.2222, 0.5476), CC3 (0.9999, 0.5476, 0.0952, 0.5476, 0.9999, 0.2222), CD45(0.6905, 0.8413, 0.1508, 0.3095, 0.6905, 0.8413), ASMA(0.0556, 0.8413, 0.3095, 0.0556, 0.2222, 0.1508), CD31(0.9999, 0.0952, 0.0952, 0.4206, 0.8413, 0.1508). Right in each are exemplar photomicrographs of the indicated marker in the indicated tumour type (scale=50 um).

FIG. 7. NALCN loss-of-function increases tumor metastasis. a, Unsupervised hierarchical clustering of P1^{K}P(GAC, n=10; lung adenocarcinoma, n=6; prostatic adenocarcinoma, n=2), V1^{KP}(IAC, n=19), Pdx1^{KP} (PAC, n=13) and P1; Pten^{Flx/Flx}; Trp53^{Flx/Fl} (P1^{PtP}) (hepatobiliary, n=3; lung adenocarcinoma, n=1) primary tumors and metastatic (liver, n=2; peritoneum, n=11; kidney, n=1; thoracic cavity, n=4; lung, n=1; lymph node, n=2) tumors. Heatmap reports enrichment of primary tumor transcriptomes in metastatic tumors. b, Exemplar ZSG⁺ metastatic tumors (met, outlined). Scale bar, 0.5 cm. c, Photomicrographs (H&E (left) and immunohistochemistry/fluorescence(right)) of the metastases in b. Scale bar, 50 μm. All enumerated metastases were evaluated using H&E (full list is given in Rahrmann et al 2022—Supplementary Table 9; n=7,076 metastases); n=59 metastases were evaluated by ZSG for IHC and n=20 metastases were evaluated by immunofluorescence. Single-channel images are shown in FIG. 15. d, Left: cumulative total number of adenocarcinoma metastases per mouse post Cre-recombination (two-tailed Mann-Whitney U-test, total tumor burden in Nalcn-deleted versus wild-type mice; Rahrmann et al 2022—Supplementary Table 9). Right: total metastases per mouse in anatomical regions. Male/female (M:F) and P3/P60 mice are shown. V1KP IAC for individual organs: liver, *P=0.0371 (Nalcn^{Flx/Flx}); kidney, *P=0.0229 (Nalcn^{Flx/Flx}); and peritoneum, *P=0.0492 (Nalcn^{+/Flx}) and **P=0.0015 (Nalcn^{Flx/Flx}). Pdx1^{KP} PAC individual organs: lung, *P=0.0328 (Nalcn^{+/Flx}); and peritoneum, **P=0.0050 (Nalcn^{+/Flx}). P1^{KP} GAC and IAC individual organs: lung, **P=0.0085 (Nalcn^{+/Flx}) and **P=0.0048 (Nalcn^{Flx/Flx}). e, Metastatic burden and organ metastases in V1^{KP}-IAC gadolinium or control treated mice. **P=0.0090, two-tailed Mann-Whitney U-test.

FIG. 8. Metastases of P1KP-GAC, V1KP-IAC and Pdx1KP-PAC. Photomicrographs of (a) P1^{KP}-GAC, (b) V1^{KP}-IAC and (c) Pdx1^{KP}-PAC metastases to the indicated tissues. Top in each, immunohistochemistry of ZsGreen staining. Bottom in each, haematoxlin and eosin (H & E) stain (scale=100 um). All enumerated metastases were evaluated by H&E (full list Rahrmann et al 2022—Supplementary Table 7; n=7,076 metastases) n=59 metastases evaluated by ZSG for IHC.

FIG. 9. NALCN loss-of-function increases nucleated CZCs in P1KP, V1KP and Pdx1KP mice. a, FACS profiles gating CZCs in blood samples of P1KP Nalcn+/+ and NalcnFlx/Flx mice (percent nucleated cells). Gating strategy is shown in FIG. 16. b, Scatter plot of CZCs (percent of total nucleated blood cells) of Prom1CreERT2/LacZ (n=397), Villin-1CreERT2 (n=162) or Pdx1Cre (n=40) mice that did, or did not, contain a primary tumor. Data are biologically independent peripheral blood samples. Bar, median. V1-Cre: *P=0.0499, ****P<0.0001; Pdx1-Cre: not significant (NS) P=0.0513, **P=0.0033; P1-Cre: **P=0.0033, ****P<0.0001; two-tailed Mann-Whitney U-test. Source data are available in Rahrmann et al 2022—Supplementary Table 13. c, Scatter plot of CZCs according to genotype and gadolinium treatment in tumor-bearing animals. Data are biologically independent peripheral blood samples. Bar, median. P1KP (n=112): *P=0.02, NS P=0.1204; V1KP (n=64): **P=0.0088, ***P=0.0004, NS P=0.4213; Pdx1 KP (n=34): *P=0.0499, **P=0.0027; two-tailed Mann-Whitney U-test. Source data are available in Rahrmann et al 2022—Supplementary Table 13. d, Representative photomicrographs of ZSG immunohistochemistry of bone marrow of mice of the indicated genotype at a minimum of 100 d post Cre-recombination. Scale, 100 μm. Three mice were evaluated for each Cre strain. e,f, FACS quantification of CZCs in P1 KP (Nalcn+/+, n=11; Nalcn+/Flx, n=4; NalcnFlx/Flx, n=6) (e) and V1 KP (Nalcn+/+, n=9; Nalcn+/Flx, n=4; NalcnFlx/Flx, n=4) (f) mice (mean±s.e.m.) from 1-week post tamoxifen induction. Source data are given in Rahrmann et al 2022—Supplementary Table 13.

FIG. 10. Nucleated CZCs in P1KP, V1KP and Pdx1KP mice are CTCs. a, UMAP of SCS profiles of CZCs (n=1,820) and PBMCs (n=559). b, Gene set enrichment from 2,086 gene sets in UMAP clusters in a. c, Coimmunofluorescence of CZCs and PBMCs in P1 KP (upper) and V1KP (lower) mice (ZSG; scale bar, 10 μm). Representative photomicrographs of 22 cells identified across n=20 blood films assessed from n=5 tumor-bearing animals. d, Autofluorescence of Pdx1KP-PAC CZC metastases in whole lung of recipient immunocompromised mouse (upper left; scale bar, 0.5 cm). Other images show H&E (representative image of 3,061 metastases evaluated) or coimmunofluorescence of metastases (representative images of 28 metastases evaluated) of P1 KP-GAC or V1 KP-IAC CZC metastases in recipient mice (scale bar, 50 μm). Single-channel images are shown in FIG. 15. e, Total metastases per organ in recipient mice injected with 25,000 CZCs. P1 KP Nalcn+/Flx PAC, n=5 mice; P1KP Nalcn+/+ GAC, n=2 mice; P1KP Nalcn+/Flx GAC, n=3 mice; V1KP Nalcn+/Flx IAC, n=2 mice; V1 KP Nalcn+/++ GdCl3 IAC, n=5 mice. Source data are given in Rahrmann et al 2022—Supplementary Table 19. f, Metastasis-free survival of immunodeficient NOD scid gamma recipient mice injected with different numbers (10,000, 1,000, 100 or 10) of P1 KP GAC or Pdx1 KP PAC CZCs (n=3 mice for each condition). ***P=0.0002 Mantel-Cox statistic. Source data are available in Rahrmann et al 2022—Supplementary Table 19.

FIG. 11. Human circulating tumour cells (CTCs) and peripheral blood mononuclear cells (PBMCs). (a) UMAP of single cell RNA sequencing (SCS) profiles of human CTCs and PBMCs (see main text for SCS sources). Genesets enriched in the indicated SCS clusters are shown with adjusted p-value for enrichment in parenthesis. (b) Heatmap of indicated gene expression from relevant genesets enriched in each cell from each cluster in (a). (c) Feature plots of exemplar genes enriched in human CTCs in (a). (d) Mouse orthologues of human genes in (c) mapped onto the UMAP of mouse CZCs and PBMCs in main FIG. 3b. (e) UMAPs of SCS profiles of common orthologues expressed in human CTCs and mouse tCZCs. (f) Enrichment of haemoglobin gene expression in UMAP shown in (e). (g) Geneset enrichments in the dotted-line enclosed, central cluster relative to the other SCS profiles is reported in (e).

FIG. 12. NALCN loss-of-function increases shedding of ntCZCs. a, ntCZCs identified in individual nontumor-bearing P1^{R}Nalcn^{+/+} (n=87), P1^{R}Nalcn^{+/Flx} (n=50) and P1^{R}Nalcn^{Flx/Flx} (n=37) mice. Bar, median. ****P<0.0001, two-tailed Mann-Whitney U-test. Source data are available in Rahrmann et al 2022—Supplementary Table 13. b, UMAP of 201,183 SCS profiles of PBMCs, tCZCs and ntCZCs as well as cells derived from the indicated normal and malignant mouse tissues. c, Coimmunofluorescence of ntCZCs and PBMCs in peripheral blood smears of P1^{R}Nalcn^{Flx/FlX} mice (ZSG; scale bar, 10 μm). Representative photomicrographs of 11 cells identified in n=20 blood films from n=4 mice. Single-channel images are shown in FIG. 15. d-f, Direct ZSG-immunofluorescence photomicrographs of ZSG'⁰ cells in lung and kidney (scale bar, 50 μm) (d), and enumerated in lung (no Cre, n=2 mice, 5 lung lobes; Nalcn+, n=3 mice, 9 lung lobes; Nalcn^{+/Flx}, n=3 mice, 8 lung lobes; Nalcn^{Flx/Flx}, n=5 mice, 12 lung lobes; NS P=0.1312, *P=0.0168, two-tailed Mann-Whitney U-test) (e) and kidney (no Cre, n=2 mice, 4 kidney sections; Nalcn^{+/+}, n=3 mice, 11 kidney sections; Nalcn^{+/Flx}, n=3 mice, 10 kidney sections; Nalcn^{Flx/Flx} n=5 mice, 18 kidney sections; ****P<0.0001, two-tailed Mann-Whitney U-test) (f). g, Organ heatmap of total numbers of ZSG'⁰ cell clusters per mouse identified in organs of recipient mice injected with P1^{R}Nalcn^{Flx/Flx} ntCZCs. h, Coimmunofluorescence of P1^{R}Nalcn^{Flx/Flx} ntCZCs (arrows) incorporated into the kidneys of recipient mice (arrows indicated ZSG⁺ cells; scale bar, 50 μm). Representative photomicrograph of n=5 ZSG rests identified in one tissue field from n=5 mice. Single-channel images are shown in FIG. 15. GLO, glomerulus. i, Confocal laser scanning microscope image of P1^{R}Nalcn^{Flx/Flx} CZCs incorporated into the renal cortex of recipient mice. Scale bar, 100 μm. Representative image of n=2 mouse kidneys assessed.

FIG. 13. NALCN loss-of-function circulating non-tumour cells (ntCZCs) resemble human and mouse CTCs and embed in distant organs. (a) Test Genesets were derived from 2,086 different tissue and cell types including bulk RNAseq of mouse normal tissues and tumours, huCTC signatures, and mouse and human intestinal stem and mature cell signatures (see Methods). (b) ZSG immunohistochemistry of aged Pdx1^{R}Nalcn^{+/+} (top left) and Pdx1^{R}Nalcn^{Flx/Flx} (bottom left) mouse lung bronchioles (scale=100 um). Right, the number of ZSG'⁰ cells/bronchiole in the lungs of Pdx1^{R}Nalcn^{+/+} (n=2 mice, 6 lung lobes, 121 bronchiole) and Pdx1^{R}Nalcn^{Flx/Flx} (n=1 mouse, 4 lung lobes, 57 bronchioles). (bar=median; **p=0.0051 two-tailed Mann-Whitney U Test). (c) Two-photon direct ZSG⁺ cell clusters detected in entire lung section of a Pdx1^{R}Nalcn^{Flx/Flx} mouse. (d) Exemplar co-immunofluorescence of tail vein injected P1^{R}Nalcn^{Flx/Flx} ntCZCs (arrows) incorporated into the organs of recipient mice (arrows indicated ZSG⁺ cells, scale bar=50 um).

FIG. 14 Organ fibrosis following conditional deletion of Nalcn at P3 in P1R mice. Fibrosis-free survival for all organs (a) or the indicated organs (b-i). P value reports the log-rank statistic (Mantel-Cox). The numbers of animals of each genotype are shown. p-values for each graph comparing P1^{R}Nalcn/and P1^{R}Nalcn+/Flx and P1^{R}Nalcn/and P1^{R}Nalcn^{Flx/Flx}, respectively: All organs (0.0664, 0.0035), Kidneys (0.0037, 0.0022), Skin (0.1195, 0.0569), Lungs(0.3791, 0.1000), Liver(0.8846, 0.7250), Stomach(0.4938, 0.4225), Small intestine(>0.9999, 0.2348), Large intestine(0.1312, 0.2655), Pancreas(0.4764, 0.7571). (j) Photomicrographs of haematoxlin and eosin (H & E) and Picro-Sirus Red stain and co-immunofluorescence of ZsGreen, alpha-smooth muscle actin (ASMA) and Dapi in kidney from P1^{R}Nalcn^{+/+} and P1^{R}Nalcn^{Flx/Flx} mice aged >400 days. Note in P1^{R}Nalcn^{Flx/Flx} kidney: extensive fibrosis below the hashed line (H & E); marked Picro-Sirius Red staining indicating extensive fibrosis; ZsGreen recombination, gross distortion of normal kidney architecture and extensive alpha-SMA expression. (k) Photomicrographs of H & E stained skin from P1^{R}Nalcn^{+/+} and P1^{R}Nalcn^{Flx/Flx} mice aged >400 days. Note in P1^{R}Nalcn^{Flx/Flx} skin: ulceration and thickening of cornified layer, marked thickening of squamous cell layer and fibrosis of dermal layer. (scale 100 um).

FIG. 15. single channel images of immunofluorescence studies of the indicated panels. All scale bars=50 μm except for FIG. 7c that are 10 μm.

FIG. 16. Standard curves generated by two ‘spike-in’ control techniques. (a) Normal peripheral blood was harvested from adult P1-KP mice with gastric adenocarcinoma. Peripheral blood mononuclear cells (PBMCs) were isolated by ficoll gradient centrifugation. ZSG+ cells were enumerated manually and spiked into fresh peripheral blood to give the final number of actual ZSG+ cells/ml (x-axis). These samples were then subject to the same FACS protocol used in all blood isolation studies to provide the observed (y-axis) quantification. The vertical dotted lines represent the 25th and 75th percentile of observed CZCs/ml recorded in Rahrmann et al 2022—Supplementary Table 11. (b) Normal PBMCs used in (a) were also spiked at the indicated percentage of total PBMCs in buffered saline and quantified in the same manner as in (a). In both graphs, the hashed black line represents the ideal curve in which expected and observed results are the same.

FIG. 17. Exemplar FACS gating strategies for isolating ZSG+ cells from peripheral blood samples. (a) Example of a tumour-bearing animal that did not have ZSG+ cells in the peripheral blood. (b) Example of a tumour-bearing animal with ZSG+ cells in the peripheral blood.

### DETAILED DESCRIPTION

The embodiments of the invention will now be further described. In the following passages, different embodiments are described. Each aspect so defined may be combined with any other aspect or aspects unless clearly indicated to the contrary.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012).

Ion channels are crucial components of cellular excitability and are involved in many diseases. The present inventors have herein demonstrated that NALCN plays a crucial role in both malignant and non-malignant cell metastasis. NALCN is a nonselective monovalent cation channel which is a sole member of a distinct branch of voltage-gated sodium and calcium channels that regulates the resting membrane potential and excitability of neurons. NALCN is expressed most abundantly in the nervous system and conducts a persistent sodium leak current that contributes to tonic neuronal excitability. The sequence of NALCN is known and may comprise the sequence provided in ENSG00000102452 (ensemble), 259232 (NCBI Entrez Gene), 19082 (HGNC), Q8IZFO (UniProtKB/Swiss-Prot), or 611549 (OMIM). In one embodiment the sequence of NALCN comprises SEQ ID NO.1. There are multiple splice variants of NALCN and the present invention extends to these variants. NALCN forms a polypeptide chain of 24 transmembrane helices (TM) that form four homologous functional repeats, also referred to as a-subunits, connected by intracellular linkers. Each functional repeat comprises voltage sensing domain, pore helices and ion selectivity filter.

It has been shown herein that loss or reduction of function of NALCN contributes to an increase in circulating tumour cell (CTC) levels and seeding of metastases. As such by identifying mutations within NALCN that correlate to NALCN loss of function it is possible to detect and/or prognose subjects likely to exhibit metastasis.

As such, in an aspect the invention relates to a method for the detection or prognosis of cancer and/or metastasis comprising:
analysing a tumour sample obtained from a subject,
determining the presence of at least one mutation within sodium leak channel (NALCN) in the tumour sample, compared to a reference sample,
determining whether the at least one mutation causes a reduction in the pore size of NALCN,
where the mutation causes a reduction in the pore size of NALCN, using the reduction in pore size to determine a risk score of cancer and/or metastasis.

The reference sample is used for comparison with said tumour sample, in order to identify the presence of a mutation in NALCN present in the tumour sample. The reference sample may be a sample obtained from a healthy subject or a sample from the subject. Where the reference sample is obtained from the subject or a healthy subject the reference sample may comprise germline DNA. A germline DNA sample may be obtained by any reasonable means. The reference sample may be obtained from a blood sample, a tissue sample, a saliva sample of a healthy subject. The reference sample may be obtained from a blood sample, a tissue sample, a saliva sample of said subject. In an embodiment the reference sample is a sample of germline DNA obtained from said subject, or a sample of germline DNA obtained from a healthy subject.

Comparison of the NALCN sequence in the tumour sample and the reference sample may be performed by sequencing. Sequencing may be performed using whole genome, whole exome, targeted exome, transcriptome, and methylome sequencing. Techniques are known in the art for comparing sequences to identify the presence of mutations, for example sequence alignment may be used.

Once the presence of at least one mutation in NALCN in the tumour sample has been identified, computational modelling may be used to determine whether at least one mutation causes a reduction in pore size of NALCN. The term “pore size” as used herein refers to the ion conducting pore of NALCN. The “pore size” may be measured in terms of the ion-selectivity filter radius and/or the gate radius. The selectivity filter radius refers to the region of the protein that confers sodium ion specificity. It is a rigid part of the structure that is shaped to only allow sodium ions to easily pass through. The ion-selectivity filter is the narrowest portion of the ion channel where amino acids (NALCN: EEKE, EKEE or EEEE) lining the filter directly interact and discriminate between ion species. In human NALCN the selectivity filter is specifically residues E280, E554, K1115, and E1389. These residues form a ring in the channel domain of the protein that constricts the channel to the exact radius of a sodium ion. The gate in the channel pore regulates ion permeation and refers to a region at the end of each S6 helix, where the channel of the protein is constricted to be closed in a depolarised state. These helices can slide open like an iris upon polarisation in order to open the channel and allow the passage of ions. Computational modelling may be performed by generating a model of NALCN within a lipid membrane and then simulating the effect of the identified mutation on the NALCN model. Techniques and software are known in the art to generate a computational model of NALCN for example using available X-ray crystallographic or cryo-EM structures of NALCN, these structures are accessible via databases such as the PDB (Protein Data Bank). In order to determine the effect of a mutation on the pore size of NALCN suitable programs are available, for example programs such as HOLE (Smart, et al., HOLE: A program for the analysis of the pore dimensions of ion channel structural models. Journal of Molecular Graphics, doi:10.1016/S0263-7855(97)00009-X (1996) which is a program that allows the analysis and visualisation of the pore dimensions of the holes through molecular structures of ion channels. There are also additional tools that may be used to calculate pore properties including CHAP (Klesse et al. CHAP: A Versatile Tool for the Structural and Functional Annotation of Ion Channel Pores. J Mol Biol. 2019; 431(17):3353-3365), CAVER (Chovancova et al. CAVER 3.0: a tool for the analysis of transport pathways in dynamic protein structures. PLoS Comput Biol. 2012; 8(10):e1002708.) and MOLE (Sehnal et al. MOLE 2.0: advanced approach for analysis of biomacromolecular channels. J Cheminform. 2013; 5(1):39).

In an aspect, the invention relates to An aspect of the invention relates to a computer-implemented method for determine a risk score of cancer and/or metastasis, the method comprising obtaining data indicating presence of at least one mutation in a sodium leak channel, NALCN, in a tumour sample, inputting the data into a computational model of NALCN that simulates effects of mutations on NALCN, determining, using the computational model, whether the at least one mutation causes a reduction in a pore size of NALCN, and outputting, when the at least one mutation is determined to cause a reduction in pore size of NALCN, a risk score of cancer and/or metastasis.

There may also be a computer device comprising at least one processor coupled to memory and arranged to perform the computer-implemented method described herein. There may also be a computer-readable storage medium comprising instructions which, when executed by a processor, causes the processor to carry out the obtaining, inputting, determining and outputting steps of the computer-implemented method described herein. The computer-readable storage medium may be, for example, but is not limited to, an electronic, magnetic, optical, electromagnetic, infrared, or semiconductor system, apparatus, or device, or any suitable combination of the foregoing.

In an aspect, the invention relates to a method for the detection or prognosis of cancer and/or metastasis comprising:
analysing a biological sample obtained from a subject, to assess the activity of sodium leak channel (NALCN),
providing a risk score of cancer and/or metastasis based on the level of activity of NALCN.

The biological sample may be a tumour sample, a blood sample, or a tissue sample. A tumour or tissue sample may be obtained via a biopsy.

As NALCN is an ion channel responsible for the resting Na+ permeability of cells the activity of NALCN may be assessed using a variety of techniques. Activity may be assessed by whole-cell electrophysiology, a fluorescence assay, a membrane potential sensing dye, and/or an ion flux assay.

In order to determine whether the activity of NALCN in the biological sample is altered the method may further comprise a step of comparing the level of activity of NALCN in the biological sample with a reference value. The reference value may be an activity measurement of NALCN obtained from a healthy subject As used herein, a “healthy subject” is defined as a subject that does not have a diagnosable cancer disease state.

In an aspect the invention relates to a method for the detection or prognosis of cancer and/or metastasis comprising:
analysing a biological sample to detect the presence of one or more mutations which correspond to a reduction of function of NALCN
providing a risk score of cancer and/or metastasis based on the presence of one or more mutations which correspond to a reduction of function of NALCN.

The NALCN protein comprises multiple domains, as such the method may detect mutations in one of the following domains; pore turret domains, voltage sensing domains or linker domains of NALCN. The linker domains may be linker domains that extend either extracellularly or intracellularly. The method may detect a mutation in one or more of the domains comprising any one of the amino acid sequences set out in SEQ ID NO. 2 to 23. The domains of NALCN and their sequences are set out in the following table:

TABLE 1

| NALCN | |
|---|---|
| Domain | Amino acid sequence |
| Full length | MLKRKQSSRVEAQPVTDFGPDESLSDN |
| NALCN (SEQ | ADILWINKPWVHSLLRICAIISVISVCMNTP |
| ID NO. 1) | MTFEHYPPLQYVTFTLDTLLMFLYTAEMIA |
| | KMHIRGIVKGDSSYVKDRWCVFDGFMVF |
| | CLWVSLVLQVFEIADIVDQMSPWGMLRIPR |
| | PLIMIRAFRIYFRFELPRTRITNILKRSGEQIWS |
| | VSIFLLFFLLLYGILGVQMFGTFTYHCVVNDT |
| | KPGNVTWNSLAIPDTHCSPELEEGYQCPPG |
| | FKCMDLEDLGLSRQELGYSFNEIGTSIFTV |
| | YEAASQEGWVFLMYRAIDSFPRWRSYFYFIT |
| | LIFFLAWLVKNVFIAVIIETFAEIRVQFQQMW |
| | GSRSSTTSTATTQMFHEDAAGGWQLVAVDV |
| | NKPQGRAPACLQKMMRSSVFHMFILSMVTV |
| | DVIVAASNYYKGENFRRQYDEFYLAEVAFTVL |
| | FDLEALLKIWCLGFTGYISSSLHKFELLLVIGTT |
| | LHVYPDLYHSQFTYFQVLRVVRLIKISPALEDF |
| | VYKIFGPGKKLGSLVVFTASLLIVMSAISLQMFC |
| | FVEELDRFTTFPRAFMSMFQILTQEGWVDVMD |
| | QTLNAVGHMWAPVVAIYFILYHLFATLILLSLFV |
| | AVILDNLELDEDLKKLKQLKQSEANADTKEKLP |
| | LRLRIFEKFPNRPQMVKISKLPSDFTVPKIRESF |
| | MKQFIDRQQQDTCCLLRSLPTTSSSSCDHSKR |
| | SAIEDNKYIDQKLRKSVFSIRARNLLEKETAVT |
| | KILRACTRQRMLSGSFEGQPAKERSILSVQHH |
| | IRQERRSLRHGSNSQRISRGKSLETLTQDHSNT |
| | VRYRNAQREDSEIKMIQEKKEQAEMKRKVQEE |
| | ELRENHPYFDKPLFIVGREHRFRNFCRVVVRAR |
| | FNASKTDPVTGAVKNTKYHQLYDLLGLVTYLDW |
| | VMIIVTICSCISMMFESPFRRVMHAPTLQIAEYV |
| | FVIFMSIELNLKIMADGLFFTPTAVIRDFGGVMDIF |
| | IYLVSLIFLCWMPQNVPAESGAQLLMVLRCLRPL |
| | RIFKLVPQMRKVVRELFSGFKEIFLVSILLLTLMLV |
| | FASFGVQLFAGKLAKCNDPNIIRREDCNGIFRI |
| | NVSVSKNLNLKLRPGEKKPGFWVPRVWANPR |
| | NFNFDNVGNAMLALFEVLSLKGWVEVRDVIIHR |
| | VGPIHGIYIHVFVFLGCMIGLTLFVGVVIANFNEN |
| | KGTALLTVDQRRWEDLKSRLKIAQPLHLPPRPDN |
| | DGFRAKMYDITQHPFFKRTIALLVLAQSVLLSVKW |
| | DVEDPVTVPLATMSVVFTFIFVLEVTMKIIAMSPA |
| | GFWQSRRNRYDLLVTSLGVVWVVLHFALLNAYT |
| | YMMGACVIVFRFFSICGKHVTLKMLLLTVVVSM |
| | YKSFFIIVGMFLLLLCYAFAGVVLFGTVKYGENIN |
| | RHANFSSAGKAITVLFRIVTGEDWNKIMHDCMVQ |
| | PPFCTPDEFTYWATDCGNYAGALMYFCSFYVIIAY |
| | IMLNLLVAIIVENFSLFYSTEEDQLLSYNDLRHFQIIW |
| | NMVDDKREGVIPTFRVKFLLRLLRGRLEVDLDKD |
| | KLLFKHMCYEMERLHNGGDVTFHDVLSMLSYRSV |
| | DIRKSLQLEELLAREQLEYTIEEEVAKQTIRMWLK |
| | KCLKRIRAKQQQSCSIIHSLRESQQQELSRFLNPPS |
| | IETTQPSEDTNANSQDNSMQPETSSQQQLLSPTL |
| | SDRGGSRQDAADAGKPQRKFGQWRLPSAP |
| | KPISHSVSSVNLRFGGRTTMKSVVCKMNPMTDAAS |
| | CGSEVKKWWTRQLTVESDESGDDLLDI |
| Voltage | INKPWVHSLLRICAIISVISVCMNTPMTFEHYPPLQYV |
| sensing | TFTLDTLLMFLYTAEMIAKMHIRGIVKGDSSYVKDRW |
| domain 1 | CVFDGFMVFCLWVSLVLQVFEIADIVDQMSPWGMLR |
| (SEQ ID NO. | IPRPLIMIRAFRIYFRFELPRTRITNILKRSGEQIWS |
| 2) | VSIFLLFFLLLYGILGVQMFGTFTY |
| Extracellular | HCVVNDTKPGNVTWNSLAIPDTHCSPELEEGYQCP |
| linker 1 (SEQ | PGFKCMDLEDLGLSRQELGYSG |
| ID NO. 3) | |
| Pore turret | FNEIGTSIFTVYEAASQEGWVFLMYRAIDSFPRWRSYF |
| domain 1 | YFITLIFFLAWLVKNVFIAVIIETFAEIRVQFQQMW |
| (SEQ ID NO. | |
| 4) | |
| Selective filter | QEG |
| (SEQ ID NO. | |
| 5) | |
| Voltage | ACLQKMMRSSVFHMFILSMVTVDVIVAASNYYKGEN |
| sensing | FRRQYDEFYLAEVAFTVLFDLEALLKIWCLGFTGYIS |
| domain 2 | SSLHKFELLLVIGTTLHVYPDLYHSQFTYFQVLRVVRL |
| (SEQ ID NO. | IKIS |
| 6) | |
| S4-S5 linker | PALEDFVYKIF |
| (SEQ ID NO. 7) | |
| Pore turret | GPGKKLGSLVVFTASLLIVMSAISLQMFCFVEELDRFTTF |
| domain 2 | PRAFMSMFQILTQEGWVDVMDQTLNAVGHMWAPVVA |
| (SEQ ID NO. | IYFILYHLFATLILLSLFVAVILDNLELD |
| 8) | |
| Selective filter | QEG |
| (SEQ ID NO. | |
| 9) | |
| DII-DIII linker | EDLKKLKQLK |
| (SEQ ID NO. | |
| 10) | |
| Voltage | EHRFRNFCRVVVRARFNASKTDPVTGAVKNTKYH |
| sensing | QLYDLLGLVTYLDWVMIIVTICSCISMMFESPFRRVMH |
| domain 3 | APTLQIAEYVFVIFMSIELNLKIMADGLFFTPTAVIRDFG |
| (SEQ ID NO. 11) | GVMDIFIYLVSLIFLCWMPQNVPAESGAQLLMVLRCL |
| | RPLRIFKLV |
| S4-S5 linker | PQMRKVVRELFS |
| (SEQ ID NO. | |
| 12) | |
| Extracellular | KLAKCNDPNIIRREDCNGIFRINVSVSKNLNLKLRPGEK |
| linker 2 (SEQ | KPGFWVPRVWANPRNFNFD |
| ID NO. 13) | |
| Pore turret | NVGNAMLALFEVLSLKGWVEVRDVIIHRVGPIHGIYIH |
| domain 3 | VFVFLGCMIGLTLFVGVVIANFNENK |
| (SEQ ID NO. | |
| 14) | |
| Selective filter | LKG |
| (SEQ ID NO. | |
| 15) | |
| DIII-DIV linker | GTALLTVDQRRWEDLKSRLKIAQPLHLPPRPDN |
| (SEQ ID NO. | |
| 16) | |
| Voltage | DGFRAKMYDITQHPFFKRTIALLVLAQSVLLSVKWDV |
| sensing | EDPVTVPLATMSVVFTFIFVLEVTMKIIAMSPAGFWQ |
| domain 4 | SRRNRYDLLVTSLGVVWVVLHFALLNAYTYMMGAC |
| (SEQ ID NO. | VIVFRFFSICGKHV |
| 17) | |
| S4-S5 linker | TLKMLLLTVVVSMYK |
| (SEQ ID NO. | |
| 18) | |
| Extracellular | FGTVKYGENINRHANFSSA |
| linker 3 (SEQ | |
| ID NO. 19) | |
| Pore turret | KAITVLFRIVTGEDWNKIMHDCM |
| domain 4 | |
| (SEQ ID NO. 20) | |
| Selective filter | GED |
| (SEQ ID NO. | |
| 21) | |
| Extracellular | VQPPFCTPDEFTYWATDCGN |
| linker 4 (SEQ | |
| ID NO. 22) | |
| C-terminal | LLSYNDLRHFQIIWNMVDDKREGVIPTFRVKFLLRL |
| domain (SEQ | LRGRLEVDLDKDKLLFKHMCYEMERLHNGGDVTF |
| ID NO. 23) | HDVLSMLSYRSVDIRKSLQLEELLAREQLEYTIEEE |
| | VAKQTIRMWLKKCLKRIRAKQQQSCSIIHSLRESQQ |
| Nalcn forward | GCCCTCAGCCCCCAAAC |
| qRTPCR | |
| oligonucleotide | |
| (SEQ ID NO. 24) | |
| Nalcn reverse | GGAAGCTGTGTCTGGCATGG |
| qRTPCR | |
| oligonucleotide | |
| (SEQ ID NO. 25) | |
| Gapdh forward | AGGTCGGTGTGAACGGATTTG |
| qRTPCR | |
| oligonucleotide | |
| (SEQ ID NO. 26) | |
| Gapdh reverse | TGTAGACCATGTAGTTGAGGTCA |
| qRTPCR | |
| oligonucleotide | |
| (SEQ ID NO. 27) | |
| Nalcn forward | ATTGTCCGTGAGATTGCTCATCACC |
| 3-primer PCR | |
| (SEQ ID NO. | |
| 28) | |
| Nalcn reverse | GCACCAGCTATATGTCCCTCTCACG |
| 3-primer PCR | |
| (SEQ ID NO. | |
| 29) | |
| Nalcn floxed | GGAAAATGACCACTTCCTAGCAGAAGC |
| allele reverse | |
| 3-primer PCR | |
| (SEQ ID NO. | |
| 30) | |

NALCN protein forms a channelosome complex within the cell membrane. The channelosome includes various proteins associated with NALCN including G-protein-coupled receptors, UNC-79, UNC-80, SL02.1, NCA localization factor-1, FAM155A and src family tyrosine kinases. As such the methods described herein may comprise further detecting a mutation in one or more of the proteins associated with NALCN. The proteins associated with NALCN in which a mutation may be detected include: M3 muscarinic receptor (M3R), UNC80, UNC79, FAM155A, Fam155B, SLO2.1, NCA localization factor-1, src family tyrosine kinases. Where a mutation is identified in a protein associated with NALCN the mutation may be identified by determining the presence of a known mutation or by determining the presence of at least one mutation within a protein associated with NALCN, compared to a reference sample, The method of the present invention may detect specific mutations within NALCN. The method may detect one or more specific mutation which correlates to a reduction in the activity of NALCN or a reduction of the pore size of NALCN. The one or more of the mutations within NALCN may be present at positions selected from, but not limited to; L588M, P573, R855, K1213, T71, P225, D1527, D416, C1348, R297, V1386, A1091, V1229, D134, T272, R43, A1157, V1036, M520, R1500, V320, V53, W1085, E1458, N1274, V1542, Y1300, R1174, H1523, F332, Q549, L999, F540, A1421, R1384, H569, Ai435, M55, R1495, C245, F110, V510, C970, E454, V273, R1556, S174, S1068, V385, S384, A401, S902, R1495, A276, R1540, L517, R295, R382, H876, F300, R164, E257, R995, G1526, D291, V1239, E1552, N1475, M55, L1553, Y1349, E323, A1044, T1281, V1007, L253, L564, F1427, V949, Q279, T539, R159, K452, R1127, V1490, G555, E62, L1461, L942, R166, P65, D952, 1322, F154, K1163, L305, R152, W1085, R143, A1444, R989, R143, R1193, D1466, M520, V1285, S52,151, E1518, E532, L1279, V1329, T57, A1378, S121, K498, R1094, V120, A88, A401, L1548, G1303, M150, D1277, E432, L1442, P1082, T1165, G1316, R1273, E128, E906, F1311, R1481, T204, T552, F389, D1527, P908, A1166, 1577, G954, G1013, P65, E1016, N1070, S980, A1217, V1503, T1320, A223, A310, R1127, D1504, D1277, E128, K1491, Q553, V511, F1250, S1374, D211, T1149, D1099, M1425, M1003, P467, R43, L222, V400, M1244, A424, F1410, G193, H39, W219, F1018, R1193, K1069, V50, R1498, K1230, S403, S1264, R995, Q238, 11433, P66, L428, D1171, A1107, S1033, 11017, K1259, M986. It has been demonstrated herein that mutations at each of these positions can result in the closure of the NALCN pore i.e. a reduction in the size of the NALCN pore and therefore a reduction in NALCN activity. It is hypothesised that these amino acid residues may be involved in regulating the opening of the NALCN pore as such mutations at one or more of these positions may result in a reduction in the pore diameter and therefore a reduction in NALCN activity. In an embodiment the method may detect one or more mutations selected from the mutations identified in Table 2. Table 6 provides further details on the mutations listed in Table 2 and how the metastatic risk was assessed.

TABLE 2
Mutations identified in NALCN and their overall metastasis risk

| | | overall |
|---|---|---|
| | nucelotide | metastasis risk |
| Mutation | change | score |
| R1481S | c.4443A>T | high |
| G1316V | c.3947G>T | high |
| A401V | c.1202C>T | high |
| L253H | c.758T>A | high |
| N1475K | c.4425C>G | high |
| V273I | c.817G>A | high |
| D134Y | c.400G>T | high |
| S1264L | c.3791C>T | low |
| K1230N | c.3690G>T | low |
| V50I | c.148G>A | low |
| M1425L | c.4273A>C | low |
| A223D | c.668C>A | low |
| V1503A | c.4508T>C | low |
| P66L | c.197C>T | medium |
| H39P | c.116A>C | medium |
| F1250S | c.3749T>C | medium |
| F389L | c.1167C>A | medium |
| L1442P | c.4325T>C | medium |
| A401T | c.1201G>A | medium |
| K498T | c.1493A>C | medium |
| V1329I | c.3985G>A | medium |
| S52P | c.154T>C | medium |
| L942S | c.2825T>C | medium |
| R1193H | c.3578G>A | high |
| T1165M | c.3494C>T | high |
| R166I | c.497G>T | high |
| K452E | c.1354A>G | high |
| R1500S | c.4500G>T | high |
| F1427L | c.4281C>A | high |
| H876R | c.2627A>G | high |
| R295H | c.884G>A | high |
| A1421V | c.4262C>T | high |
| V1386I | c.4156G>A | high |
| L564V | c.1690C>G | high |
| V1007A | c.3020T>C | high |
| L1553P | c.4658T>C | high |
| R995H | c.2984G>A | high |
| R382W | c.1144C>T | high |
| S902F | c.2705C>T | high |
| S384F | c.1151C>T | high |
| V385I | c.1153G>A | high |
| R1556G | c.4666A>G | high |
| L999V | c.2995C>G | high |
| R1174I | c.3521G>T | high |
| V1542M | c.4624G>A | high |
| V320A | c.959T>C | high |
| S403G | c.1207A>G | low |
| L222S | c.665T>C | low |
| V400M | c.1198G>A | low |
| R43C | c.127C>T | low |
| Q553L | c.1658A>T | low |
| D1277A | c.3830A>C | low |
| T1320M | c.3959C>T | low |
| A1217T | c.3649G>A | low |
| S980L | c.2705C>T | low |
| A424D | c.1271C>A | medium |
| D211Y | c.631G>T | medium |
| E1518K | c.4552G>A | medium |
| E128G | c.383A>G | medium |
| R1273I | c.3818G>T | medium |
| E432D | c.1296A>C | medium |
| D1277N | c.3829G>A | medium |
| S121C | c.362C>G | medium |
| I51S | c.152T>G | medium |
| R989Q | c.2966G>A | medium |
| I322F | c.964A>T | medium |
| D952N | c.2854G>A | medium |
| K1069N | c.3207G>C | high |
| D1099N | c.3295G>A | high |
| A310T | c.928G>A | high |
| F1311L | c.3933T>G | high |
| G1303D | c.3908G>A | high |
| R152Q | c.455G>A | high |
| E62K | c.184G>A | high |
| G1526S | c.4576G>A | high |
| S1068A | c.3202T>G | high |
| V1229F | c.3685G>T | high |
| L588M | c.1762C>A | high |
| Q279H | c.837G>T | high |
| E257G | c.770A>G | high |
| E1458K | c.4372G>A | high |
| A1044V | c.3131C>T | high |
| Y1349H | c.4045T>C | high |
| F300S | c.899T>C | high |
| E454K | c.1360G>A | high |
| C970Y | c.734G>T | high |
| V510F | c.1528G>T | high |
| R1495Q | c.4484G>A | high |
| R1384Q | c.4151G>A | high |
| V53D | c.158T>A | high |
| M520V | c.1558A>G | high |
| T272I | c.815C>T | high |
| A1091V | c.3272C>T | high |
| C1348W | c.4044T>G | high |
| F1018I | c.3052T>A | low |
| M986I | c.2958G>T | low |
| I1017N | c.3050T>A | low |
| D1171N | c.3511G>A | low |
| Q238H | c.714G>C | low |
| R1498C | c.4492C>T | low |
| G193E | c.578G>A | low |
| M1244T | c.3731T>C | low |
| P467R | c.1400C>G | low |
| K1491T | c.4472A>C | low |
| R1127C | c.3379C>T | low |
| N1070K | c.3210C>A | medium |
| L305V | c.913C>G | medium |
| G954S | c.2860G>A | medium |
| P908L | c.2723C>T | medium |
| L1548F | c.4644G>T | medium |
| A88T | c.262G>A | medium |
| V120A | c.359T>C | medium |
| T57R | c.170C>G | medium |
| V1285I | c.3853G>A | medium |
| F154S | c.461T>C | medium |
| G555R | c.1663G>A | medium |
| R159Q | c.476G>A | medium |
| R143W | c.427C>T | high |
| L1461F | c.4381C>T | high |
| R1540W | c.4618C>T | high |
| F540S | c.1619T>C | high |
| T1281M | c.3842C>T | high |
| E327K | c.979G>A | high |
| E323K | c.967G>A | high |
| E1552K | c.4654G>A | high |
| V1239A | c.3716T>C | high |
| R1495W | c.4483C>T | high |
| S174L | c.521C>T | high |
| M55I | c.165G>A | high |
| Y1300S | c.3899A>C | high |
| R43H | c.128G>A | high |
| D416N | c.1246G>A | high |
| R855Q | c.2564G>A | high |
| V511A | c.1532T>C | low |
| D1527N | c.4579G>A | medium |
| R995C | c.2983C>T | medium |
| R146Q | c.437G>A | medium |
| G1013S | c.3037G>A | medium |
| R1193C | c.3577C>T | medium |
| R143Q | c.428G>A | medium |
| P65S | c.193C>T | medium |
| V1490I | c.4468G>A | medium |
| T539M | c.1616C>T | medium |
| WT | | low |

In an embodiment the method comprises a further step of identifying the stage of the cancer based on the one or more mutations that are identified. The method may comprises a further step of identifying the risk of metastasis based on the one or more mutations that are identified. In an embodiment the method comprises a further step of determining/selecting a treatment. Thus, we also describe a method for determining a treatment for a subject the method comprising one or more of the methods described above and further comprising the further step of determining a treatment. The treatment may be selected from any suitable anti-cancer treatment and/or anti-metastatic treatment. The treatment may be selected from chemotherapy, hormone therapy, immunotherapy, radiation therapy, stem cell therapy, surgery or targeted therapies such as small molecule therapy, antibody therapy, checkpoint inhibitors or CAR-T therapy. Such treatments are known in the art. It will be appreciated that there are various types of immunotherapies such as immune checkpoint inhibitors, oncolytic virus therapy, T cell therapy and cancer vaccines. The appropriate therapy may be selected.

The method of the present invention allows the detection or prognosis of cancer. In an embodiment the cancer is selected from gastric cancer, gastric adenocarcinoma, colorectal cancer, lung cancer, non-small cell lung cancer, lung adenocarcinoma, lung squamous cell carcinoma, bone cancer, pancreatic cancer, colon cancer, colorectal cancer, skin cancer, cancer of the head or neck, head and neck squamous cell carcinoma, melanoma, uterine cancer, ovarian cancer, rectal cancer, cancer of the anal region, stomach cancer, testicular cancer, breast cancer, brain cancer, hepatocellular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, kidney cancer, sarcoma of soft tissue, cancer of the urethra, cancer of the bladder, renal cancer, thymoma, urothelial carcinoma leukemia, prostate cancer, prostatic adenocarcinoma mesothelioma, adrenocortical carcinoma, lymphomas, such as such as Hodgkin's disease, non-Hodgkin's, and multiple myelomas. In an embodiment the cancer is selected from gastric, intestinal or pancreatic cancer.

The methods of detection or prognosis of cancer and/or metastasis comprise a step of determining a risk score of cancer/metastasis. The risk score may be based on determining the reduction in NALCN pore size due to the mutation that has been identified within NALCN. The inventors have shown herein that it is possible to determine the reduction in pore size caused by mutation via computational modelling. A larger reduction in the NALCN pore size correlates to a larger reduction in NALCN activity and therefore a higher risk of cancer and/or metastasis. The reduction in NALCN pore size may be calculated by determining the difference in size of the ion-selectivity filter radius in the NALCN variant comprising a mutation compared to the wild-type NALCN filter radius. The reduction in NALCN pore size may be calculated by determining the difference in size of the gate radius in the NALCN variant comprising a mutation compared to the wild-type NALCN gate radius. The risk score may also be determined based on the presence of specific mutations identified wherein a risk of cancer and/or metastasis has been associated with the specific mutation. Where the method for the prognosis of cancer and/or metastasis comprises determining a risk score based on the activity of NALCN a higher risk score is associated with a larger reduction in activity of NALCN

As an example, the risk score may be calculated using the following steps:
the wildtype NALCN structure is taken and mutated to induce the amino acid changes observed in the subject. This mutation process may be performed multiple times for example three times
the mutant structure is minimized using a small molecular dynamics simulation, wherein Newtonian physics is applied to the structure to allow atoms to adapt to the induced change. This same process is applied to each of the mutant structures if more than one is used.
the pore radius is calculated for the minimized structure. Where multiple structures are used, the pore radius is calculated and averaged to provide an estimate of the pore closure. Pore radius is calculated using a program such as HOLE or another suitable program. As an example, the program HOLE calculates pore radius by growing spheres incrementally along the channel axis until they contact the protein, resulting in an estimation of the profile of the channel.

The risk score of cancer can then be used to determine a likelihood of a cancer or metastatic disease state. A “likelihood of a cancer or metastatic disease state” means that the probability that the cancer disease state exists in the subject specimen is about 50% or more, for example 60%, 70%, 80% or 90%.

“Prognosis” refers, e.g., to overall survival, long term mortality, and disease free survival. In one embodiment, long term mortality refers to death within 5 years after diagnosis of lung cancer.

In an aspect the invention relates to a method for determining the activity of NALCN comprising:
analysing a biological sample to detect one or more mutations identified in Table 2,
wherein the presence of one or more mutation identified in Table 2 indicates reduced activity of NALCN.

In an embodiment the method of the invention may comprise analysing a biological sample to detect one or more mutations identified in any one of Tables 3, 4, or 5.

The methods of the invention comprise detecting mutations within NALCN. In an embodiment the mutations are detected via whole genome, whole genome, whole exome, targeted exome, transcriptome, and methylome sequencing. In an embodiment the mutations may be detected using one or more techniques selected from; allele-specific polymerase chain reaction (PCR), high resolution melting curve analysis, genomic sequencing fluorescence in situ hybridization (FISH); comparative genomic hybridization (CGH), Restriction fragment length polymorphism RELP), amplification refractory mutation system (ARMS), reverse transcriptase PCR (RT-PCR), real-time PCR, multiplex ligation-dependent probe amplification (MLPA), denaturing gradient gel electrophoresis (DGGE), single strand conformational polymorphism (SSCP), chemical cleavage of mismatch (CCM), protein truncation test (PTT), or oligonucleotide ligation assay (OLA).

The methods of detection or prognosis of cancer and/or metastasis, or the methods for determining NALCN activity are generally performed in vitro or ex vivo. The methods require a biological sample that has been obtained from a subject which is then analysed. As such, the steps of analysis are performed outside the human body i.e. in vitro or ex vivo. The biological sample may be a tumour sample. The sample may be obtained from a subject via a biopsy or during surgery to remove said tumour. The biological sample may have been processed after removal from the subject, for example the sample may be cyro-preserved.

The biological sample obtained from the subject may be a subject comprising somatic mutation for example the sample may be a tissue sample or a tumour sample.

An aspect the invention relates to a kit comprising reagents for the detection of one or more mutations in NALCN, wherein the mutation correlates to a reduction in the activity of NALCN and/or a reduction in the pore size of NALCN and optionally instructions for use. In an embodiment the kit comprises reagents for the detection of one or more mutations in NALCN identified in Table 2. In an embodiment kit comprises reagents for the detection of one or more mutations in NALCN identified in Table 6, identified either by the amino acid change or the nucleotide mutation.

In an aspect the invention relates to a composition comprising reagents for the detection of one or more mutations in NALCN, wherein the mutation correlates to a reduction in the activity of NALCN and/or a reduction in the pore size of NALCN. In an embodiment the composition comprises reagents for the detection of one or more mutations in NALCN identified in Table 2. In an embodiment composition comprises reagents for the detection of one or more mutations in NALCN identified in Table 6, identified either by the amino acid change or the nucleotide mutation.

In an embodiment the kit or composition of the invention comprises reagents suitable for carrying out whole genome, whole exome, targeted exome, transcriptome, and methylome sequencing. Preferably the reagents are suitable for performing allele-specific polymerase chain reaction (PCR), high resolution melting curve analysis, genomic sequencing fluorescence in situ hybridization (FISH); comparative genomic hybridization (CGH), Restriction fragment length polymorphism RELP), amplification refractory mutation system (ARMS), reverse transcriptase PCR (RT-PCR), real-time PCR, multiplex ligation-dependent probe amplification (MLPA), denaturing gradient gel electrophoresis (DGGE), single strand conformational polymorphism (SSCP), chemical cleavage of mismatch (CCM), protein truncation test (PTT), or oligonucleotide ligation assay (OLA).

In an embodiment the kit comprises reagents for the detection of one or more of the mutations in NALCN that have been identified as high-risk mutations for metastasis. NALCN mutations identified as being a high risk of metastasis are set out in the below table:

TABLE 3
Mutations in NALCN associated with a high-risk of metastasis

| | | overall |
|---|---|---|
| | nucelotide | metastasis risk |
| Mutation | change | score |
| R1481S | c.4443A>T | high |
| G1316V | c.3947G>T | high |
| A401V | c.1202C>T | high |
| L253H | c.758T>A | high |
| N1475K | c.4425C>G | high |
| V273I | c.817G>A | high |
| D134Y | c.400G>T | high |
| R1193H | c.3578G>A | high |
| T1165M | c.3494C>T | high |
| R166I | c.497G>T | high |
| K452E | c.1354A>G | high |
| R1500S | c.4500G>T | high |
| F1427L | c.4281C>A | high |
| H876R | c.2627A>G | high |
| R295H | c.884G>A | high |
| A1421V | c.4262C>T | high |
| V1386I | c.4156G>A | high |
| L564V | c.1690C>G | high |
| V1007A | c.3020T>C | high |
| L1553P | c.4658T>C | high |
| R995H | c.2984G>A | high |
| R382W | c.1144C>T | high |
| S902F | c.2705C>T | high |
| S384F | c.1151C>T | high |
| V385I | c.1153G>A | high |
| R1556G | c.4666A>G | high |
| L999V | c.2995C>G | high |
| R1174I | c.3521G>T | high |
| V1542M | c.4624G>A | high |
| V320A | c.959T>C | high |
| K1069N | c.3207G>C | high |
| D1099N | c.3295G>A | high |
| A310T | c.928G>A | high |
| F1311L | c.3933T>G | high |
| G1303D | c.3908G>A | high |
| R152Q | c.455G>A | high |
| E62K | c.184G>A | high |
| G1526S | c.4576G>A | high |
| S1068A | c.3202T>G | high |
| V1229F | c.3685G>T | high |
| L588M | c.1762C>A | high |
| Q279H | c.837G>T | high |
| E257G | c.770A>G | high |
| E1458K | c.4372G>A | high |
| A1044V | c.3131C>T | high |
| Y1349H | c.4045T>C | high |
| F300S | c.899T>C | high |
| E454K | c.1360G>A | high |
| C970Y | c.734G>T | high |
| V510F | c.1528G>T | high |
| R1495Q | c.4484G>A | high |
| R1384Q | c.4151G>A | high |
| V53D | c.158T>A | high |
| M520V | c.1558A>G | high |
| T272I | c.815C>T | high |
| A1091V | c.3272C>T | high |
| C1348W | c.4044T>G | high |
| R143W | c.427C>T | high |
| L1461F | c.4381C>T | high |
| R1540W | c.4618C>T | high |
| F540S | c.1619T>C | high |
| T1281M | c.3842C>T | high |
| E327K | c.979G>A | high |
| E323K | c.967G>A | high |
| E1552K | c.4654G>A | high |
| V1239A | c.3716T>C | high |
| R1495W | c.4483C>T | high |
| S174L | c.521C>T | high |
| M55I | c.165G>A | high |
| Y1300S | c.3899A>C | high |
| R43H | c.128G>A | high |
| D416N | c.1246G>A | high |
| R855Q | c.2564G>A | high |

In an embodiment the kit comprises reagents for detecting one or more of the mutations in NALCN that have been identified as medium-risk mutations for metastasis. NALCN mutations identified as being a medium risk of metastasis are set out in the below table:

TABLE 4
Mutations in NALCN associated with a medium-risk of metastasis

| | | overall |
|---|---|---|
| | nucelotide | metastasis risk |
| Mutation | change | score |
| P66L | c.197C>T | medium |
| H39P | c.116A>C | medium |
| F1250S | c.3749T>C | medium |
| F389L | c.1167C>A | medium |
| L1442P | c.4325T>C | medium |
| A401T | c.1201G>A | medium |
| K498T | c.1493A>C | medium |
| V1329I | c.3985G>A | medium |
| S52P | c.154T>C | medium |
| L942S | c.2825T>C | medium |
| A424D | c.1271C>A | medium |
| D211Y | c.631G>T | medium |
| E1518K | c.4552G>A | medium |
| E128G | c.383A>G | medium |
| R1273I | c.3818G>T | medium |
| E432D | c.1296A>C | medium |
| D1277N | c.3829G>A | medium |
| S121C | c.362C>G | medium |
| I51S | c.152T>G | medium |
| R989Q | c.2966G>A | medium |
| I322F | c.964A>T | medium |
| D952N | c.2854G>A | medium |
| N1070K | c.3210C>A | medium |
| L305V | c.913C>G | medium |
| G954S | c.2860G>A | medium |
| P908L | c.2723C>T | medium |
| L1548F | c.4644G>T | medium |
| A88T | c.262G>A | medium |
| V120A | c.359T>C | medium |
| T57R | c.170C>G | medium |
| V1285I | c.3853G>A | medium |
| F154S | c.461T>C | medium |
| G555R | c.1663G>A | medium |
| R159Q | c.476G>A | medium |
| D1527N | c.4579G>A | medium |
| R995C | c.2983C>T | medium |
| R146Q | c.437G>A | medium |
| G1013S | c.3037G>A | medium |
| R1193C | c.3577C>T | medium |
| R143Q | c.428G>A | medium |
| P65S | c.193C>T | medium |
| V1490I | c.4468G>A | medium |
| T539M | c.1616C>T | medium |

In an embodiment the kit comprises reagents for detecting one or more of the mutations in NALCN that have been identified as low-risk mutations for metastasis. NALCN mutations identified as being a low risk of metastasis are set out in the below table:

TABLE 5
Mutations in NALCN associated with a low-risk of metastasis

| | | overall |
|---|---|---|
| | nucelotide | metastasis risk |
| Mutation | change | score |
| S1264L | c.3791C>T | low |
| K1230N | c.3690G>T | low |
| V50I | c.148G>A | low |
| M1425L | c.4273A>C | low |
| A223D | c.668C>A | low |
| V1503A | c.4508T>C | low |
| S403G | c.1207A>G | low |
| L222S | c.665T>C | low |
| V400M | c.1198G>A | low |
| R43C | c.127C>T | low |
| Q553L | c.1658A>T | low |
| D1277A | c.3830A>C | low |
| T1320M | c.3959C>T | low |
| A1217T | c.3649G>A | low |
| S980L | c.2705C>T | low |
| F1018I | c.3052T>A | low |
| M986I | c.2958G>T | low |
| I1017N | c.3050T>A | low |
| D1171N | c.3511G>A | low |
| Q238H | c.714G>C | low |
| R1498C | c.4492C>T | low |
| G193E | c.578G>A | low |
| M1244T | c.3731T>C | low |
| P467R | c.1400C>G | low |
| K1491T | c.4472A>C | low |
| R1127C | c.3379C>T | low |
| V511A | c.1532T>C | low |

The kit of the present invention may be provided as a panel of reagents designed to detect one or more of the NALCN mutations set out in Table 2. The panel of reagents may be designed to detect one or more of the mutations identified as indicating a high risk of metastasis as set out in Table 3. The panel of reagents may be designed to detect one or more of the mutations identified as indicating a medium risk of metastasis as set out in Table 4. The panel of reagents may be designed to detect one or more of the mutations identified as indicating a low risk of metastasis as set out in Table 5.

In an embodiment the tumour sample or biological sample is obtained from a subject such as a mammal, preferably a human.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present disclosure, including methods, as well as the best mode thereof, of making and using this disclosure, the following examples are provided to further enable those skilled in the art to practice this disclosure. However, those skilled in the art will appreciate that the specifics of these examples should not be read as limiting on the invention, the scope of which should be apprehended from the claims and equivalents thereof appended to this disclosure. Various further aspects and embodiments of the present disclosure will be apparent to those skilled in the art in view of the present disclosure.

All documents mentioned in this specification are incorporated herein by reference in their entirety, including references to gene accession numbers, scientific publications and references to patent publications.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

The term “comprising” or “comprises” where used herein means including the component(s) specified but not to the exclusion of the presence of other components. The term “consisting essentially of” or “consists essentially of” means including the components specified but excluding other components except for materials present as impurities, unavoidable materials present as a result of processes used to provide the components and the like.

The term “consisting of” or “consists of” means including the components specified but excluding other components.

Whenever appropriate, depending upon the context, the use of the term “comprises” or “comprising” may also be taken to include the meaning “consists essentially of” or “consisting essentially of”, and also may also be taken to include the meaning “consists of” or “consisting of”.

The optional features set out herein may be used either individually or in combination with each other where appropriate and particularly in the combinations as set out in the accompanying claims. The optional features for each aspect or exemplary embodiment of the invention, as set out herein are also applicable to all other aspects or exemplary embodiments of the invention, where appropriate. In other words, the skilled person reading this specification should consider the optional features for each aspect or exemplary embodiment of the invention as interchangeable and combinable between different aspects and exemplary embodiments.

The invention is further illustrated in the following non-limiting examples.

### EXAMPLES

Intestinal cancers, including those of the stomach, are thought to arise from stem cells⁷-9; but how oncogenic mutations transform intestinal stem cells to produce invasive cancer remains unclear. The inventors have shown previously that Prominin1 (Prom1) marks basal stem cells in gastric antral glands, and that their lineage forms adenocarcinomas in Prom1^{CreERT2/LacZ}; Kras^{G12D}; Trp53^{Flx/Flx} (P1^{KP}) mice following expression of mutant-Kras^{G12D} and deletion of Trp53⁷. Prom1⁺, but not Prom1⁻, cells isolated from P1^{KP} gastric adenocarcinomas (P1^{KP}-GAC) propagated these tumours readily as allografts in immunocompromised mice; suggesting that Prom1⁺ P1^{KP}-GAC cells are the malignant counterparts of antral gland basal stem cells.

### Example 1. Nalcn Loss-of-Function is a Feature of Advanced Cancer

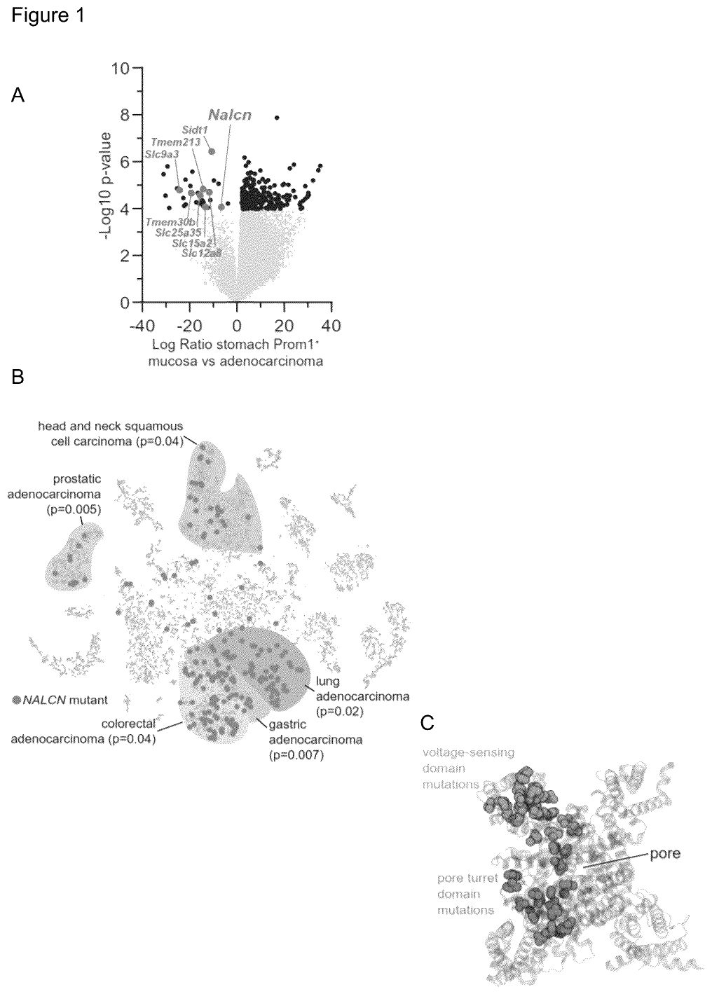

To understand better how antral gland basal stem cells are corrupted during transformation, we compared their transcriptomes with those of Prom1⁺ P1^{KP}-GAC cells. Ion channels and solute carriers were enriched among genes downregulated in Prom1⁺ P1^{KP}-GAC cells (adjusted p-value=1.7e⁻³; FIG. 1a. Review of 10,022 human cancers within The Cancer Genome Atlas showed that non-synonymous mutations in NALCN are enriched among gastric (n=43/422; dN/dS ratio, p=0.007) and colorectal adenocarcinomas (n=45/528, p=0.04; FIG. 1b)^{5,6}. Mapping these mutated residues on the cryo-electron microscope structure of NALCN, embedded and relaxed within a 575-POPC lipid bilayer in silico^{3,10,11}, revealed significant spatial-clustering within the pore turret and voltage sensing domains that regulate channel opening (p=0.03; FIG. 1c). HOLE analysis¹²—that estimates ion channel pore radius size-performed on the end frame of an equilibrium molecular dynamics simulation of membrane embedded NALCN, predicts that 76% (n=224/295) of these mutations occlude the NALCN selectivity filter, and so will close the channel^{2,3}(FIG. 1d). Among 221 patients for whom both disease stage and NALCN mutation status were available^{13,14}, NALCN mutations predicted to cause the greatest pore closure were enriched in the most advanced cancers (FIG. 1e). Further, human GACs in which NALCN was mutated, upregulated genes expressed during epithelial-mesenchymal transition (EMT, p-value=1.26e⁻⁹)—a feature of invasive cancer¹⁵.

As a first step to test if Nalcn regulates cancer progression, we altered its function in P1^{KP}-GAC cells using genetic (Nalcn-shRNA and NALCN-cDNA lentiviral transduction) or chemical (Nalcn channel blocker, gadolinium chloride [GdCl₃]⁴) approaches. Whole-cell voltage-clamp analysis of P1^{KP}-GAC cells showed a linear GdCl₃ sensitive current to voltage steps in the ±80 mV range as previously reported⁴. This current was eliminated in Nalcn^{shRNA} transduced P1^{KP}-GAC cells. Decreasing Nalcn function in P1^{KP}-GAC cells increased their proliferation in vitro and conferred an EMT morphology and transcriptome (adjusted p-value=5.29e⁻⁶) on orthotopic tumour allografts of these cells¹⁶. Conversely, increasing P1^{KP}-GAC cell NALCN expression, increased the GdCl3-sensitive current, decreased proliferation, and produced a striking hyper-epithelialized morphology in allografts.

### Example 2. Loss of Nalcn Promotes Cancer Metastasis

To study how Nalcn loss-of-function impacts cancer initiation and progression in intact tissues, we generated mice harboring a conditional Nalcn allele in which exons 5 and 6 of the gene were flanked by loxP sites (Nalcn^{Flx};). These mice were bred with P1^{KP}, Vilin1-Cre^{ERT2}; Kras^{G12D}; Trp53^{Flx/Flx} (V1^{KP}) or Pdx1-Cre; Kras^{G12D}; Trp53^{Flx/+} (Pdx1^{KP}) mice to produce equivalent numbers of male and female mice that were either Nalcn-wild-type (Nalcn+), Nalcn^{+/Flx} or Nalcn^{Flx/Flx} (total n=551;). All mice carried the Rosa26-ZsGreen (Rosa26^{ZSG}) lineage tracing allele. Cancers in V1^{KP} and Pdx1^{KP} mice are restricted by Cre expression to the intestine^{17,18} and pancreas^{19,20}, respectively. Prom1^{CreERT2/LacZ} is expressed by a variety of stem/progenitor cells and induces tumours of the small intestine, liver, lung, salivary glands, prostate, uterus, skin, and stomach in P1^{KP} mice^{7,8}. Since tissues can display age-dependent susceptibility to transformation⁷ we activated Cre-recombination in P1^{KP} and V1^{KP} mice using tamoxifen at postnatal day (P) 3 or P60. Mice displaying signs of tumour development were euthanised and subject to whole-body macro- and microscopic autopsy. As expected, V1KP (n=127/141) and Pdx1^{KP} (n=55/55) mice developed intestinal and pancreatic tumours, respectively. P1^{KP} mice developed tumours in the stomach (n=49/269), small intestine (n=59/269) and other sites (n=108/269)^{7,18,20}: 99% (n=212/214) of P1^{KP} mice developed a single primary cancer. Neither age of induction, sex or Nalcn status altered significantly the site, type, size or incidence of primary tumours, or tumour-free survival in these mouse models. Thus, Nalcn function does not appear to impact the capacity of Kras and Trp53 oncogenic mutations to transform tissues.

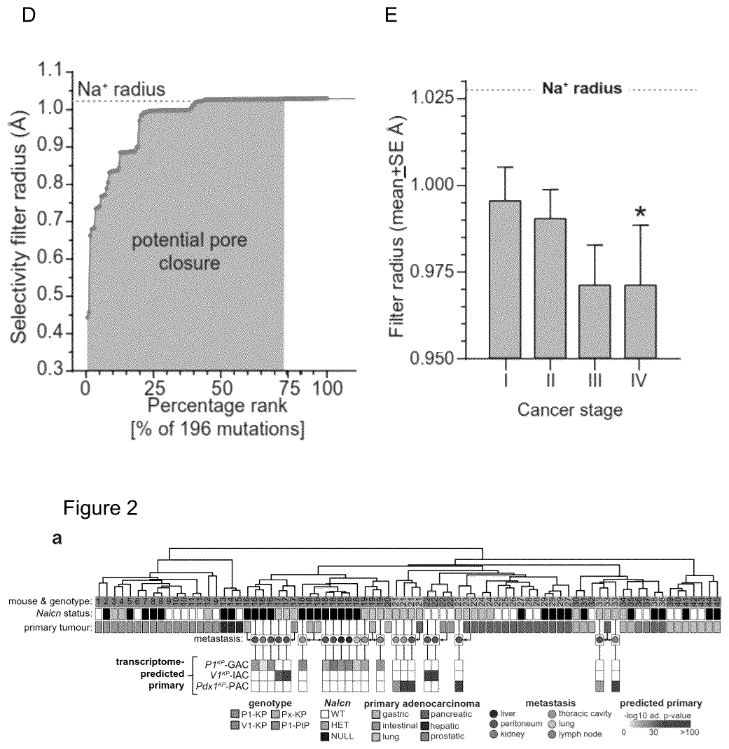

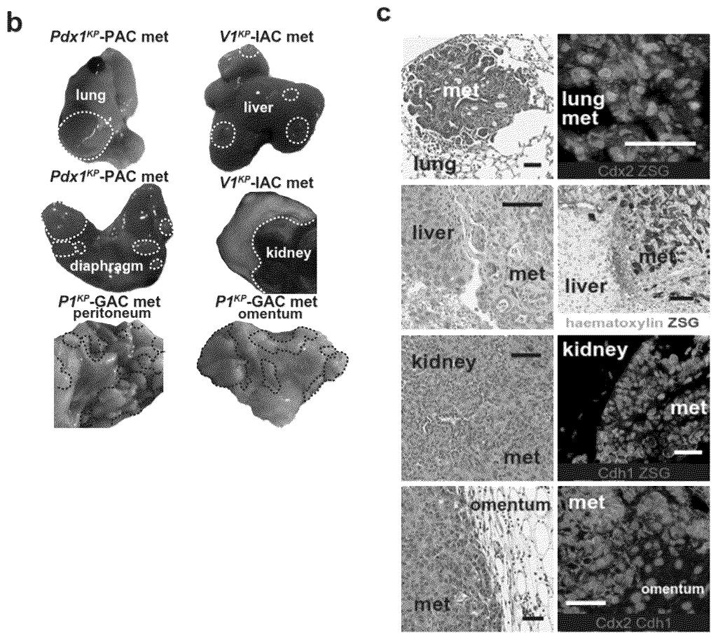

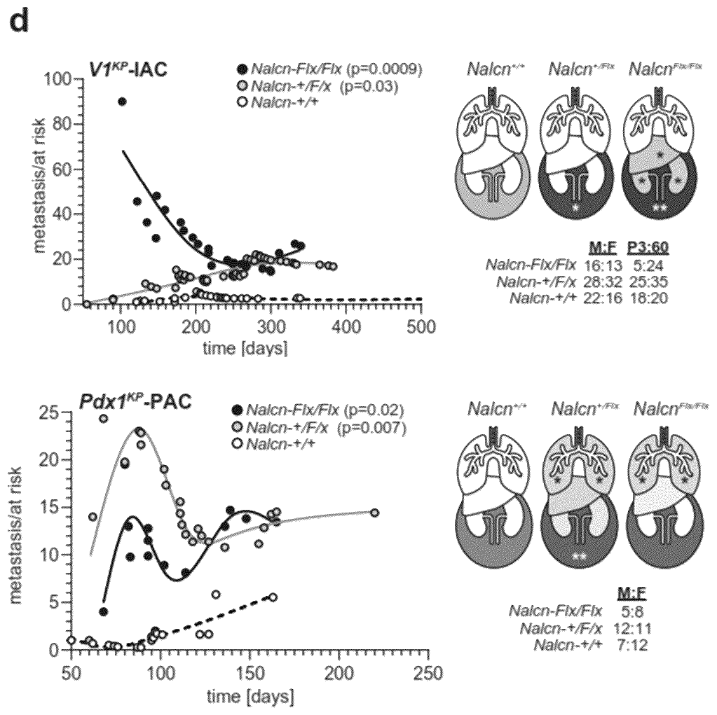

However, hetero- or homozygous deletion of Nalcn dramatically increased tumour metastasis to the peritoneum, retroperitoneum, liver, lymph nodes, lungs and/or kidneys in P1^{KP}, V1^{KP} and Pdx1^{KP} mice (FIG. 2a-c). Metastatic and primary tumours were readily distinguished from one another by: expert pathologist, blinded histology review; co-segregation of ‘matched’ primary and secondary tumour transcriptomes by unsupervised hierarchical clustering; and highly-selective enrichment within metastatic tumour transcriptomes of histology-predicted primary tumour genesets (FIGS. 2a and c). Intestinal adenocarcinomas (IACs) in V1KP Nalcn^{+/+} mice (n=27 mice) and pancreatic adenocarcinomas (PACs) in Pdx1^{KP} Nalcn^{+/+} mice (n=19 mice), produced 2.8±4.9SE and 5.5±4.0SE metastases/mouse, respectively (FIG. 2d). In stark contrast, these same tumours in V1^{KP} Nalcn^{+/Flx} (n=51), V1^{KP} Nalcn^{Flx/Flx} (n=26), Pdx1^{KP} Nalcn^{+/Flx} (n=23), and Pdx1^{KP} Nalcn^{Flx/Flx} (n=13) mice produced 16.2±5.7SE (Mann-Whitney, p=0.03 relative to Nalcn^{+/+}), 26.0±10.18SE (p=0.0009), 15.0±3.62SE (p=0.007), and 13.5±5.01SE (p=0.02) metastases/mouse, respectively. Nalcn deletion from V1^{KP}-IACs increased metastasis in particular to the peritoneum, kidneys and liver, while Nalcn deletion from Pdx1^{KP}-PACs increased metastasis to the peritoneum and lungs (FIG. 2d). Similar patterns of IAC and GAC metastatic burden were observed among 80 P1^{KP} mice that developed these, but not other, cancers: P1^{KP} Nalcn^{+/+} (11.6±3.45SE metastases/mice), P1^{KP} Nalcn^{+/Flx} (42.2±11.23SE metastases/mice) and P1^{KP} Nalcn^{Flx/Flx} (40.24.0±15.51SE metastases/mice): Nalcn loss significantly increased metastasis to the lungs and peritoneum in these mice (FIG. 2d). To validate further Nalcn-loss-of-function as a driver of cancer metastasis, we treated additional cohorts of V1^{KP} Nalcn^{+/+} (n=37), V1^{KP} Nalcn^{Flx/+} (n=17) and V1^{KP} Nalcn^{Fxl/Flx} (n=8) mice with the Nalcn channel blocker gadolinium chloride (2pg/kg/week up to 30 weeks). IACs developing in gadolinium treated V1^{KP} Nalcn^{+/+} mice (n=28) produced 18.3±5.94SE metastases/mice relative to 2.8±4.9SE in controls (p=0.02; FIG. 2e). Notably, gadolinium did not increase IAC metastasis in either V1^{KP} Nalcn^{Flx/+} or V1^{KP} Nalcn^{Flx/Flx} mice, confirming that the agent induced metastasis by blocking Nalcn-mediated currents.

### Example 3. Loss of Nalcn Increases the Number of Circulating Tumour Cells

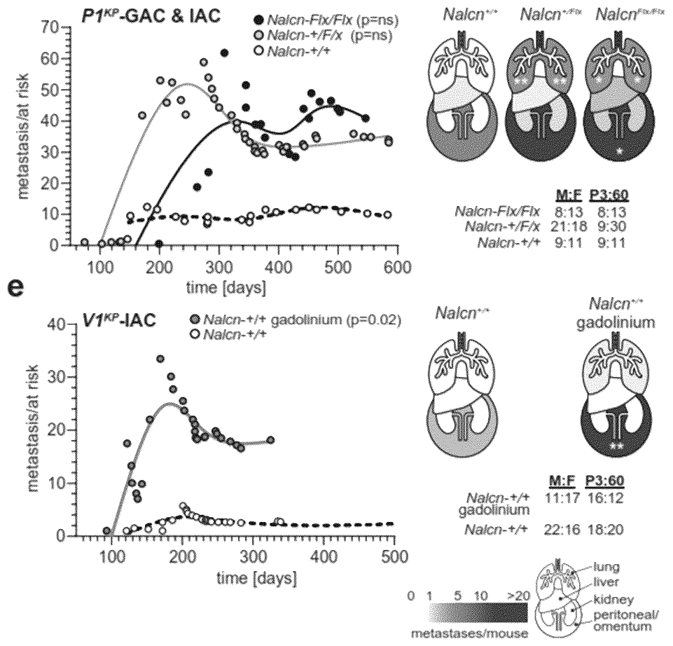

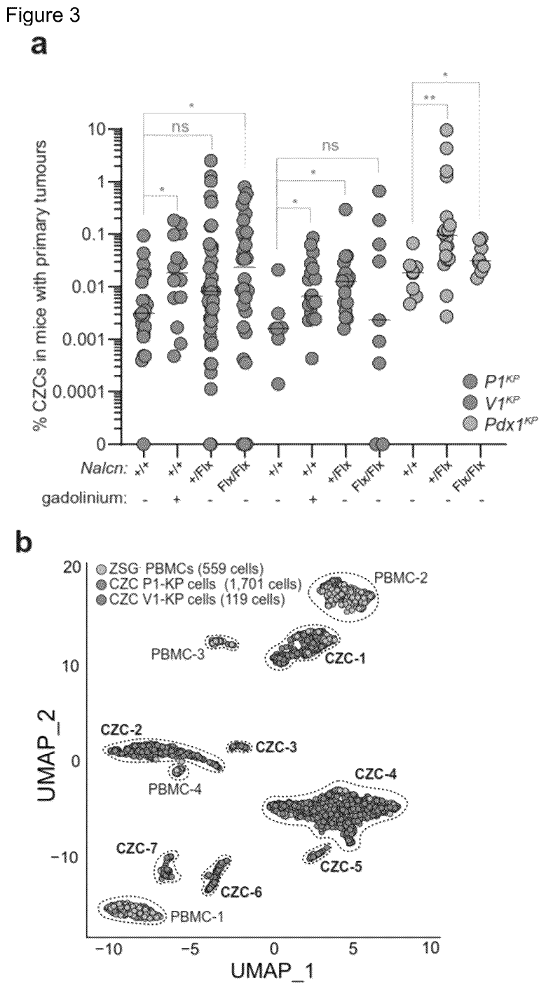

Since loss of Nalcn function increased metastasis and enriched primary tumour transcriptomes with genesets expressed by human circulating tumour cells (CTCs;), we reasoned that loss of Nalcn function might increase the release of CTCs from primary tumours into the peripheral blood: CTCs are shed from tumours as precursors of metastatic disease²¹. Nucleated circulating ZSG⁺ cells (CZCs) were quantified by fluorescence-activated cell sorting (FACS) from the peripheral blood of Prom1^{CreERT2/LacZ} (n=₃₃₇ mice), Villin-1^{CreER} (n=121 mice), or Pdx1^{Cre} (n=40 mice) mice carrying the ROSA^{ZSG} allele and various combinations of oncogenic and Nalcn^{Flx} alleles. Following blood sampling, all mice underwent whole body autopsy. An average of 4.5e³±1.1 SE CZCs/ml of blood (0.078%±0.02SE total cells) were isolated from all mice after an average of 296±9.8SE days following Cre-recombination( ). Across all three Cre-lines, the number of CZCs was highly correlated with both the presence of a primary tumour and the total number of metastases (multiple linear regression, T=10.43, p<0.0001;), independent of mouse sex or age of induction. Nalcn deletion, or gadolinium treatment, increased significantly the level of CZCs in tumour bearing P1^{KP}, V1^{KP} and Pdx1^{KP} mice (FIG. 3a). Since neither Prom1^{CreERT2/LacZ}, Villin-1^{CreERT2}. or Pdx1^{Cre} recombine haematopoeitic cells in the bone marrow, then these data suggest strongly that CZCs are CTCs shed from primary tumours through a process regulated by Nalcn.

To better understand the origin of CZCs, we generated single cell RNA sequence (SCS) profiles of CZCs isolated from mice with P1^{KP}-GAC (n=1,701 cells) or V1KP-IAC (n=119), as well as peripheral blood mononuclear cells (PBMCs, n=559), and compared these with published SCS profiles of human breast, lung, pancreatic and prostate CTCs (n=360) and PBMCs (n=500)²²⁻²⁷. Human CTCs comprised three overlapping clusters, that were readily resolved from PBMCs: ‘huCTC1’ (enriched with epithelial [adjusted p-value=1.0e⁻²⁶] and dendritic cell [adjusted p-value=0.003] genesets); huCTC3 (CD71⁺ erythroid cell enriched [adjusted p-value=1.9e⁻⁴³]); and huCTC2 (sharing profiles of huCTC1 and 3). huCTC1-3 expressed p-globin (HBB)—a survival factor for human CTCs²⁴—as well as HBA1, HBA2, and HBD. Mouse CZCs formed seven clusters whose transcriptomes closely matched huCTC1 (mCZC2-5), huCTC2 (mCZC2-7) and huCTC3 (mCZC6 and 7), and included orthologues of HBA1, HBA2 (Hba-a1, Hba-a2), HBB (Hbb-bs, Hbb-bt), ANXA2 and LGALS3, as well as genes expressed in normal and malignant stomach and small intestine (FIG. 3c). Co-immunofluorescence of peripheral blood smears taken from mice with V1KP-IAC and P1^{KP}-GAC confirmed CZC expression of Hba-a2, Lgals3, and epithelial cell markers (Krt80, Cdh1) and Cdx2 that marks intestinal epithelium (FIG. 3d). PBMCs did not express these markers but did express markers of PBMCs e.g., Cd45.

To test directly if CZCs are CTCs, we injected separate aliquots of 25,000 CZCs isolated from mice with Pdx1^{KP}-PAC, P1^{KP}-GAC or V1^{KP}-IAC into the tail veins of eight immunocompromised mice. Within 75 days, all mice developed respiratory distress and contained numerous ZSG⁺ metastases in the lungs, liver, kidneys and peritoneum (FIGS. 3e and f). Thus, CZCs include CTCs that recapitulate the transcriptome of human CTCs and are shed into the peripheral blood through a process regulated by Nalcn.

### Example 4. Nalcn Regulates Solid Tissue Cell-Shedding Independent of Cancer

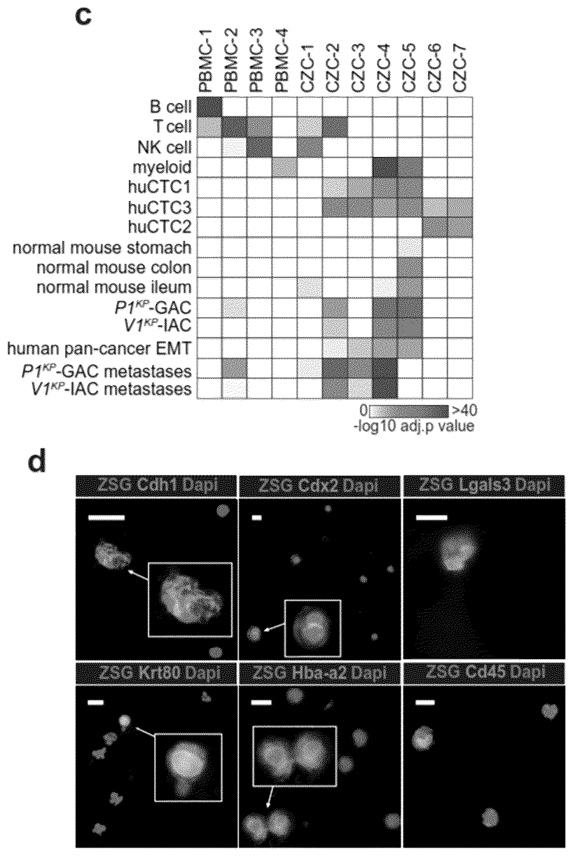

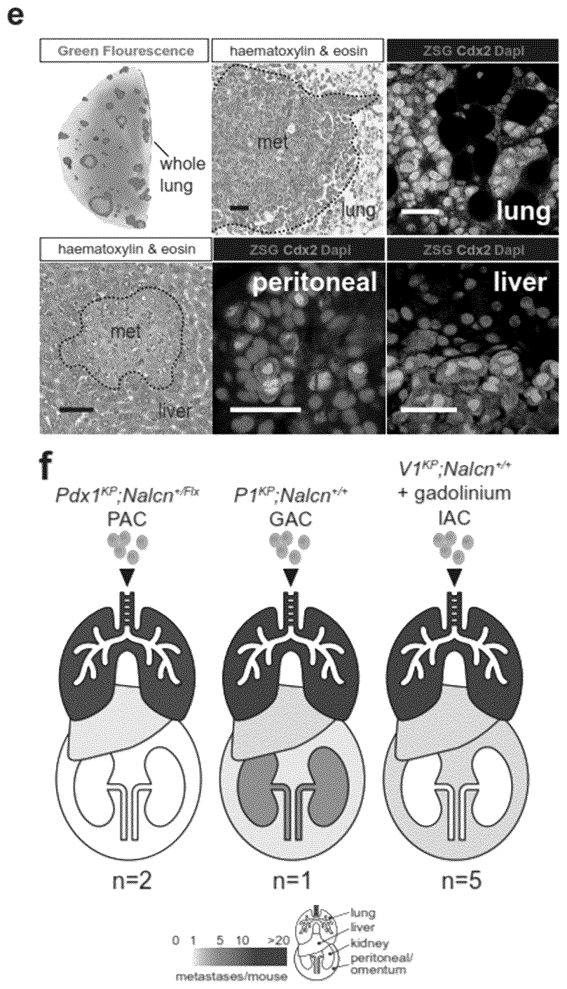

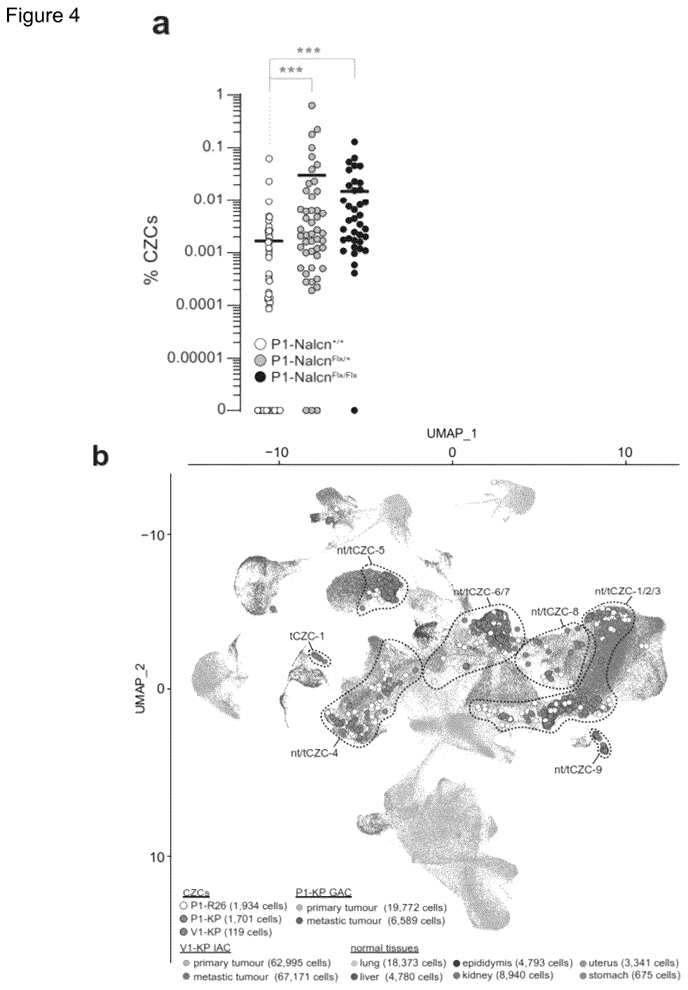

Preventing CTC shedding into the peripheral blood could stop metastasis; but disentangling this process from the complex cascade of tumourigenesis has proved challenging. To test if Nalcn regulates cell shedding from solid tissues independent of tumourigenesis, we looked for CZCs in the peripheral blood of Prom1^{creERT2/LacZ}; Rosa26^{ZSG}; Nalcn^{+/+} (P1^{R}Nalcn^{+/+}n=87), P1^{R}Nalcn^{+/Flx} (n=48) and P1^{R}Nacn^{Flx/Flx} (n=37) mice that lacked oncogenic alleles and never developed tumours ( ). Remarkably, CZCs were readily isolated from the peripheral blood of these mice, and deletion of Nalcn increased the numbers of these cells significantly—to a degree similar to that seen in tumour bearing animals (FIGS. 3a and 4a). SCS profiles of CZCs isolated from non-tumour-bearing (ntCZCs) mice co-clustered with CZCs from tumour bearing animals (tCZCs) and with IAC and GAC metastases SCSs (FIG. 4b). The great majority of tCZCs and ntCZCs SCSs did not cluster with profiles generated from primary IACs, GACs, or normal lung, liver, small intestine, stomach, kidney, uterus or epididymis cells (FIG. 4b). Similar to human CTCs¹, the SCS profiles of tCZCs and ntCZCs were highly-enriched for genesets expressed by gastric and small intestinal stem/progenitor cells (tCZC1 nt/tCZC1-4), huCTC-1 (tCZC1, nt/tCZC8 and 9), huCTC-2 (nt/tCZC4-9) and huCTC-3 (nt/tCZC8 and 9). Co-immunofluorescence of blood smears confirmed that both ntCZCs and tCZCs share markers of huCTCs, including Hba-a1 (FIGS. 3d and 4c).

To understand the fate of CZCs in non-tumour bearing mice, we injected separate aliquots of 25,000 CZCs isolated from P1^{R}Nalcn^{Flx/Flx} mice into the tail veins of six immunocompromised mice. All recipient mice remained clinically well after an average of 100 days but contained numerous ZSG⁺/Cdh1⁺/Icam1⁺ donor-cell clusters within their lungs, liver, kidneys and peritoneum at a frequency similar to metastatic tumours formed by tail vein injections of tCZCs (FIG. 3f, 4d-f). ntCZCs appeared to incorporate into, and/or form component parts of, apparently normal recipient organs—the most extreme example being their incorporation into glomeruli, vessels and/or tubules of the kidney (FIG. 4d-f). Thus, Nalcn regulates cell shedding from solid tissues independent of cancer, divorcing this process from tumourigenesis and unmasking an oncogene-independent metastatic pathway.

### Example 5. Nalcn-Blockade Causes Gadolinium-Induced Systemic Fibrosis

While P1^{R}Nalcn^{+/Flx} (n=118) and P1^{R}Nalcn^{Flx/Flx} (n=112) mice did not develop tumours, whole body autopsy of these mice revealed increasing fibrosis of the kidneys and skin—that are sites of Prom1^{CreERT2/LacZ} driven recombination⁷—relative to P1^{R}Nalcn^{+/+} (n=65) mice. Nalcn deletion did not increase fibrosis of the liver, lungs, pancreas, stomach or intestines. This pathology arose after ≥400 days and replicated that of gadolinium-induced systemic fibrosis (GISF, previously called nephrogenic systemic fibrosis)—a debilitating condition manifested by the development of severe cutaneous and systemic fibrosis following the administration of gadolinium-based contrast agents (GBCA)²⁸. Thus, our data directly implicate gadolinium-blockade of NALCN as the mechanism underpinning GISF.

### Discussion

Most patients with cancer die as a result of metastasis—the process by which cancer cells spread from the primary tumour to other organs in the body. Current understanding of metastasis is predicated on the idea that oncogenic mutations drive a cascade of events in which stem-cell like cancer cells leave the primary tumour, enter the blood stream, and travel to distant sites where they form new malignant growths^{1,29}. If correct, this model requires the presence of a primary tumour at some stage in the disease history, and assumes that the process is abnormal and unique to malignancy. By demonstrating that a single ion channel, NALCN, regulates cell trafficking from both non-malignant and malignant tissues to distant organs, we provide important new insights to the metastatic process and possible explanations for long-standing enigmatic observations.

Developing anti-metastatic therapies has proven difficult since potential therapeutic targets in primary tumours that drive metastases e.g., mutant oncoproteins, have proved hard to find¹. By divorcing the process of CTC shedding from ‘upstream’ tumourigenesis, our data unmask Nalcn function, and thereby the manipulation (depolarization) of resting membrane potential, as a promising new approach to block metastasis. Gadolinium-blockade of Nalcn increased the abundance of tCZCs in our mice; therefore, drugs capable of re-opening the channel might be effective anti-metastatic drugs. Precedent for this approach is provided by drugs that open the chloride-ion channel mutated in the disease cystic fibrosis³⁰ .

A model in which metastases always descend from a primary tumour is hard to reconcile with the observation that metastases can emerge many years after removal of a localised cancer³¹ and that up to 5% of patients with metastases lack an apparent primary tumour³². Loss of Nalcn function in our mice caused an abundant and persistent shedding of cells that embed in distant organs, even in the absence of a primary tumour. Since human epithelial tissues contain fields of phenotypically normal cells that harbour oncogenic mutations^{33,34}, then loss of NALCN function in these cells could provide a source of CTCs that form metastases in the absence of a primary tumour, or long after a primary tumour has been removed from within the field of mutant cells. It is likely that such cells would need to acquire additional mutations to form tumours at the metastatic site, compatible with the relative rarity of these phenomena. Our data may also explain why CTCs have been found in the bone marrow of patients who lack metastases. While these cells could represent ‘dormant’ CTCs as previously suggested²⁹, equivalent to ntCZCs in our mice, they may be shed from non-transformed epithelia that have lost NALCN function, but not gained the ability to form metastatic tumours.

Our observations also raise important questions: ‘How does loss of Nalcn function promote cell shedding?’ And, since we observed CZCs in P1^{R}Nalcn^{+/+} mice, albeit at lower levels than in Nalcn deleted animals, ‘Is epithelial cell trafficking a normal phenomenon that is corrupted in cancer?’ Since Nalcn loss-of-function promoted an EMT phenotype and transcriptome in tumours and CTCs in our mice, Nalcn may regulate gene transcription in a manner similar to that of calcium-ion channels³⁵: the calcium pump PMCA4 was reported to regulate an EMT transcriptome in gastric cancer cells³⁶. Further work will uncover the role of epithelial cell trafficking in normal tissue maintenance or other disease states.

Our observation that deletion of Nalcn replicated GISF in the kidneys and skin of aged animals pinpoint Nalcn-channel blockade as the likely mechanism underpinning this debilitating condition. Since P1^{KP} mice succumbed to cancer well before the onset of organ fibrosis in P1^{R} mice, and Nalcn deletion in P1^{R} mice did not induce stomach, intestine, pancreas, lung or liver fibrosis-principal sites of primary and metastatic tumours in P1^{KP} mice-then fibrosis is unlikely to contribute to metastasis in Nalcn-deleted mice. However, since limited exposure to gadolinium can induce GISF in humans, it is a note of concern that gadolinium-contrast imaging of cancer patients could accelerate metastasis.

### REFERENCES FOR EXAMPLES 1 TO 5

1 Ganesh, K. & Massagué, J. Targeting metastatic cancer. Nature medicine 27, 34-44, doi:10.1038/s41591-020-01 195-4 (2021).
2 Chua, H. C., Wulf, M., Weidling, C., Rasmussen, L. P. & Pless, S. A. The NALCN channel complex is voltage sensitive and directly modulated by extracellular calcium. Science Advances, doi:10.1126/sciadv.aaz3154 (2020).
3 Kschonsak, M. et al. Structure of the human sodium leak channel NALCN. Nature, doi:10.1038/s41586-020-2570-8 (2020).
4 Lu, B. et al. The Neuronal Channel NALCN Contributes Resting Sodium Permeability and Is Required for Normal Respiratory Rhythm. Cell 129, 371-383, doi:10.1016/j.cell.2007.02.041 (2007).
5 Martincorena, I. et al. Universal Patterns of Selection in Cancer and Somatic Tissues. Cell, doi:10.1016/j.cell.2017.09.042 (2017).
6 Weinstein, J. N. et al. The Cancer Genome Atlas Pan-Cancer analysis project. Nature Genetics 45, 1113-1120, doi:10.1038/ng.2764 (2013).
7 Zhu, L. et al. Multi-organ Mapping of Cancer Risk The generative capacity of an organ's stem cells determines the life-long risk for developing cancer in that organ. Multi-organ Mapping of Cancer Risk. Cell, doi:10.1016/j.cell.2016.07.045 (2016).
8 Zhu, L. et al. Prominin 1 marks intestinal stem cells that are susceptible to neoplastic transformation. Nature, doi:10.1038/nature07589 (2009).
9 Barker, N. et al. Crypt stem cells as the cells-of-origin of intestinal cancer. Nature 457, 608-611, doi:10.1038/nature07602 (2009).
Newport, T. D., Sansom, M. S. P. & Stansfeld, P. J. The MemProtMD database: A resource for membrane-embedded protein structures and their lipid interactions. Nucleic acids research, doi:10.1093/nar/gky1047 (2019).
11 Stansfeld, P. J. et al. MemProtMD: Automated Insertion of Membrane Protein Structures into Explicit Lipid Membranes. Structure, doi:10.1016/j.str.2015.05.006 (2015).
12 Smart, O. S., Neduvelil, J. G., Wang, X., Wallace, B. A. & Sansom, M. S. P. HOLE: A program for the analysis of the pore dimensions of ion channel structural models. Journal of Molecular Graphics, doi:10.1016/S0263-7855(97)00009-X (1996).
13 Gao, J. et al. Integrative analysis of complex cancer genomics and clinical profiles using the cBioPortal. Science Signaling, doi:10.1126/scisignal.2004088 (2013).
14 Tate, J. G. et al. COSMIC: the Catalogue Of Somatic Mutations In Cancer. Nucleic acids research 47, D941-d947, doi:10.1093/nar/gky1015 (2019).
15 Koplev, S., Lin, K., Dohlman, A. B. & Ma'ayan, A. Integration of pan-cancer transcriptomics with RPPA proteomics reveals mechanisms of epithelial-mesenchymal transition. PLoS computationalbiology 14, e1005911, doi:10.1371/journal.pcbi.1005911 (2018).
16 Cristescu, R. et al. Molecular analysis of gastric cancer identifies subtypes associated with distinct clinical outcomes. Nature medicine 21, 449-456, doi:10.1038/nm.3850 (2015).
17 El Marjou, F. et al. Tissue-specific and inducible Cre-mediated recombination in the gut epithelium. Genesis, doi:10.1002/gene.20042 (2004).
18 Jackstadt, R. et al. Epithelial NOTCH Signaling Rewires the Tumor Microenvironment of Colorectal Cancer to Drive Poor-Prognosis Subtypes and Metastasis. Cancer Cell, doi:10.1016/j.ccell.2019.08.003 (2019).
19 Magnuson, M. A. & Osipovich, A. B. Pancreas-specific Cre driver lines and considerations for their prudent use. Cell metabolism 18, 9-20, doi:10.1016/j.cmet.2013.06.011 (2013).
20 Hingorani, S. R. et al. Preinvasive and invasive ductal pancreatic cancer and its early detection in the mouse. Cancer Cell, doi:10.1016/S1535-6108(03)00309-X (2003).
21 Pantel, K. & Alix-Panabieres, C. in Nature Reviews Clinical Oncology (2019).
22 Miyamoto, D. T. et al. RNA-Seq of single prostate CTCs implicates noncanonical Wnt signaling in antiandrogen resistance. Science (New York, N.Y.) 349, 1351-1356, doi:10.1126/science.aab0917 (2015).
23 Ting, D. T. et al. Single-cell RNA sequencing identifies extracellular matrix gene expression by pancreatic circulating tumor cells. Cell reports 8, 1905-1918, doi:10.1016/j.celrep.2014.08.029 (2014).
24 Zheng, Y. et al. Expression of p-globin by cancer cells promotes cell survival during blood-borne dissemination. Nature Communications 8, 14344, doi:10.1038/ncomms14344 (2017).
25 Ebright, R. Y. et al. Deregulation of ribosomal protein expression and translation promotes breast cancer metastasis. Science (New York, N.Y.) 367, 1468-1473, doi:10.1126/science.aay0939 (2020).
26 Jordan, N. V. et al. HER2 expression identifies dynamic functional states within circulating breast cancer cells. Nature 537, 102-106, doi:10.1038/nature19328 (2016).
27 Dimitrov-Markov, S. et al. Discovery of New Targets to Control Metastasis in Pancreatic Cancer by Single-cell Transcriptomics Analysis of Circulating Tumor Cells. Molecular cancer therapeutics 19, 1751-1760, doi:10.1158/1535-7163.Mct-19-1166 (2020).
28 Todd, D. J. & Kay, J. Gadolinium-Induced Fibrosis. Annual review of medicine 67, 273-291, doi:10.1146/annurev-med-063014-124936 (2016).
29 Massagué, J. & Obenauf, A. C. Metastatic colonization by circulating tumour cells. Nature 529, 298-306, doi:10.1038/nature17038 (2016).
30 Habib, A. R. et al. A Systematic Review of the Clinical Efficacy and Safety of CFTR Modulators in Cystic Fibrosis. Scientific reports 9, 7234, doi:10.1038/s41598-019-43652-2 (2019).
31 Pantel, K., Brakenhoff, R. H. & Brandt, B. Detection, clinical relevance and specific biological properties of disseminating tumour cells. Nature Reviews Cancer 8, 329-340, doi:10.1038/nrc2375 (2008).
32 Kolling, S. et al. “Metastatic Cancer of Unknown Primary” or “Primary Metastatic Cancer” ?Frontiers in Oncology 9, doi:10.3389/fonc.2019.01546 (2020).
33 Martincorena, I. et al. Tumor evolution. High burden and pervasive positive selection of somatic mutations in normal human skin. Science (New York, N.Y.) 348, 880-886, doi:10.1126/science.aaa6806 (2015).
34 Martincorena, I. et al. Somatic mutant clones colonize the human esophagus with age. Science (New York, N.Y.) 362, 911-917, doi:10.1126/science.aau3879 (2018).
35 Barbado, M., Fablet, K., Ronjat, M. & De Waard, M. Gene regulation by voltage-dependent calcium channels. Biochimica et biophysica acta 1793, 1096-1104, doi:10.1016/j.bbamcr.2009.02.004 (2009).
36 Wang, T. et al. The calcium pump PMCA4 prevents epithelial-mesenchymal transition by inhibiting NFATc1-ZEB1 pathway in gastric cancer. Biochimica et biophysica acta. Molecular cell research 1867, 118833, doi:10.1016/j.bbamcr.2020.118833 (2020).

TABLE 6

| | | | | | | tumour stage | | |
|---|---|---|---|---|---|---|---|---|
| | | | | | | (Neoplasm | Filter | |
| | | | | | | Disease Stage | radius | |
| | | | | | | American Joint | (WT Filet | % of WT |
| | nucleotide | NALCN | Domain | tumour | | Committee on | radius = | selective |
| Mutation | change | domain | position | type | cancer | Cancer Code | 1.0279) | filter |
| R1481S | c.4443A > T | CTD | CTD | STAD | Papillary Stomach | STAGE I | 1.02765 | 99.9756786 |
| | | | | | Adenocarcinoma | | | |
| G1316V | c.3947G > T | VSD4 | 4 | COADREAD | Colorectal | STAGE I | 1.02758 | 99.9688686 |
| | | | | | Adenocarcinoma | | | |
| A401V | c.1202C > T | VSD2 | 2 | COADREAD | Colorectal | STAGE I | 0.99638 | 96.9335539 |
| | | | | | Adenocarcinoma | | | |
| L253H | c.758T > A | ECL | ECL | EAD | Esophageal | STAGE I | 1.00017 | 97.3022668 |
| | | | | | Adenocarcinoma | | | |
| N1475K | c.4425C > G | CTD | CTD | STAD | Diffuse Type | STAGE I | 0.99794 | 97.0853196 |
| | | | | | Stomach | | | |
| | | | | | Adenocarcinoma | | | |
| V273I | c.817G > A | PD1 | 1 | USC | Uterine Serous | STAGE I | 0.99361 | 96.6640724 |
| | | | | | Carcinoma/Uterine | | | |
| | | | | | Papillary Serous | | | |
| | | | | | Carcinoma | | | |
| D134Y | c.400G > T | VSD1 | 1 | COADREAD | Colorectal | STAGE I | 0.77086 | 74.9936764 |
| | | | | | Adenocarcinoma | | | |
| S1264L | c.3791C > T | VSD4 | 4 | COADREAD | Colorectal | STAGE I | 1.02855 | 100.063236 |
| | | | | | Adenocarcinoma | | | |
| K1230N | c.3690G > T | VSD4 | 4 | COADREAD | Colorectal | STAGE I | 1.02852 | 100.060317 |
| | | | | | Adenocarcinoma | | | |
| V50I | c.148G > A | S4-S5 | 1 | UEC | Uterine | STAGE I | 1.02848 | 100.056426 |
| | | LINKER | | | Endometriod | | | |
| | | | | | Carcinoma | | | |
| M1425L | c.4273A > C | PD4 | 4 | COADREAD | Colorectal | STAGE I | 1.02829 | 100.037941 |
| | | | | | Adenocarcinoma | | | |
| A223D | c.668C > A | ECL | ECL | COADREAD | Colorectal | STAGE I | 1.02804 | 100.01362 |
| | | | | | Adenocarcinoma | | | |
| V1503A | c.4508T > C | CTD | CTD | COADREAD | Colorectal | STAGE I | 1.028 | 100.009729 |
| | | | | | Adenocarcinoma | | | |
| P66L | c.197C > T | VSD1 | 1 | EAD | Esophageal | STAGE I | 1.02862 | 100.070045 |
| | | | | | Adenocarcinoma | | | |
| H39P | c.116A > C | VSD1 | 1 | EAD | Esophageal | STAGE I | 1.0284 | 100.048643 |
| | | | | | Adenocarcinoma | | | |
| F1250S | c.3749T > C | VSD4 | 4 | STAD | Stomach | STAGE I | 1.02818 | 100.02724 |
| | | | | | Adenocarcinoma | | | |
| F389L | c.1167C > A | VSD2 | 2 | LIVER | liver | STAGE I | 1.0277 | 99.9805429 |
| L1442P | c.4325T > C | PD4 | 4 | EAC | Esophageal | STAGE I | 1.02752 | 99.9630314 |
| | | | | | Adenocarcinoma | | | |
| A401T | c.1201G > A | VSD2 | 2 | COADREAD | Colorectal | STAGE I | 1.02734 | 99.94552 |
| | | | | | Adenocarcinoma | | | |
| K498T | c.1493A > C | S4-S5 | 1 | ESCC | Esophageal | STAGE I | 1.02721 | 99.9328729 |
| | | LINKER | | | Squamous Cell | | | |
| | | | | | Carcinoma | | | |
| V1329I | c.3985G > A | S4-S5 | 3 | COADREAD | Colorectal | STAGE I | 1.02717 | 99.9289814 |
| | | LINKER | | | Adenocarcinoma | | | |
| S52P | c.154T > C | VSD1 | 1 | EAD | Esophageal | STAGE I | 1.02707 | 99.9192529 |
| | | | | | Adenocarcinoma | | | |
| L942S | c.2825T > C | VSD3 | 3 | COADREAD | Colorectal | STAGE I | 1.02591 | 99.8064014 |
| | | | | | Adenocarcinoma | | | |
| R1193H | c.3578G > A | DIII-IV | 3 | COADREAD | Colorectal | STAGE II | 1.02843 | 100.051561 |
| | | LINKER | | | Adenocarcinoma | | | |
| T1165M | c.3494C > T | PD3 | 3 | USC | Uterine Serous | STAGE II | 1.02755 | 99.96595 |
| | | | | | Carcinoma/Uterine | | | |
| | | | | | Papillary Serous | | | |
| | | | | | Carcinoma | | | |
| R166I | c.497G > T | VSD1 | 1 | BC | Breast Invasive | STAGE II | 1.02598 | 99.8132114 |
| | | | | | Ductal Carcinoma | | | |
| K452E | c.1354A > G | VSD2 | 2 | COADREAD | Colorectal | STAGE II | 1.02161 | 99.3880728 |
| | | | | | Adenocarcinoma | | | |
| R1500S | c.4500G > T | CTD | CTD | EAD | Esophageal | STAGE II | 0.83552 | 81.2841716 |
| | | | | | Adenocarcinoma | | | |
| F1427L | c.4281C > A | PD4 | 4 | COADREAD | Colorectal | STAGE II | 1.01214 | 98.4667769 |
| | | | | | Adenocarcinoma | | | |
| H876R | c.2627A > G | S1NA- | 2 | COADREAD | Colorectal | STAGE II | 0.99746 | 97.0386224 |
| | | S1NB | | | Adenocarcinoma | | | |
| R295H | c.884G > A | PD1 | 1 | COADREAD | Colon | STAGE II | 0.99742 | 97.034731 |
| | | | | | Adenocarcinoma | | | |
| A1421V | c.4262C > T | PD4 | 4 | COADREAD | Colorectal | STAGE II | 0.88747 | 86.3381652 |
| | | | | | Adenocarcinoma | | | |
| V1386I | c.4156G > A | PD4 | 4 | COADREAD | Colorectal | STAGE II | 0.76691 | 74.6093978 |
| | | | | | Adenocarcinoma | | | |
| L564V | c.1690C > G | PD2 | 2 | ESCC | Esophageal | STAGE II | 1.00509 | 97.7809125 |
| | | | | | Squamous Cell | | | |
| | | | | | Carcinoma | | | |
| V1007A | c.3020T > C | S4-S5 | 3 | COADREAD | Colorectal | STAGE II | 0.99851 | 97.150501 |
| | | LINKER | | | Adenocarcinoma | | | |
| L1553P | c.4658T > C | CTD | CTD | STAD | Intestinal | STAGE II | 0.99804 | 97.0950482 |
| | | | | | Type Stomach | | | |
| | | | | | Adenocarcinoma | | | |
| R995H | c.2984G > A | VSD3 | 3 | COADREAD | Colorectal | STAGE II | 0.99771 | 97.0629439 |
| | | | | | Adenocarcinoma | | | |
| R382W | c.1144C > T | VSD2 | 2 | COADREAD | Mucinous | STAGE II | 0.99745 | 97.0376496 |
| | | | | | Adenocarcinoma | | | |
| | | | | | of the Colon | | | |
| | | | | | and Rectum | | | |
| S902F | c.2705C > T | VSD3 | 3 | COADREAD | Colorectal | STAGE II | 0.9965 | 96.9452281 |
| | | | | | Adenocarcinoma | | | |
| S384F | c.1151C > T | VSD2 | 2 | STAD | Stomach | STAGE II | 0.99637 | 96.932581 |
| | | | | | Adenocarcinoma | | | |
| V385I | c.1153G > A | VSD2 | 2 | COADREAD | Colon | STAGE II | 0.99614 | 96.9102053 |
| | | | | | Adenocarcinoma | | | |
| R1556G | c.4666A > G | CTD | CTD | COADREAD | Colorectal | STAGE II | 0.9949 | 96.789571 |
| | | | | | Adenocarcinoma | | | |
| L999V | c.2995C > G | VSD3 | 3 | STAD | Diffuse Type | STAGE II | 0.88673 | 86.2661738 |
| | | | | | Stomach | | | |
| | | | | | Adenocarcinoma | | | |
| R1174I | c.3521G > T | DIII-IV | 3 | STAD | Gastric | STAGE II | 0.88526 | 86.1231537 |
| | | LINKER | | | Adenocarcinoma | | | |
| V1542M | c.4624G > A | CTD | CTD | COADREAD | Colorectal | STAGE II | 0.88483 | 86.0813309 |
| | | | | | Adenocarcinoma | | | |
| V320A | c.959T > C | PD1 | 1 | COADREAD | Colon | STAGE II | 0.83589 | 81.3201673 |
| | | | | | Adenocarcinoma | | | |
| S403G | c.1207A > G | VSD2 | 2 | COADREAD | Colorectal | STAGE II | 1.02854 | 100.052263 |
| | | | | | Adenocarcinoma | | | |
| L222S | c.665T > C | ECL | ECL | ESCC | Esophageal | STAGE II | 1.02832 | 100.04086 |
| | | | | | Squamous Cell | | | |
| | | | | | Carcinoma | | | |
| V400M | c.1198G > A | VSD2 | 2 | COADREAD | Rectal | STAGE II | 1.02832 | 100.04086 |
| | | | | | Adenocarcinoma | | | |
| R43C | c.127C > T | VSD1 | 1 | COADREAD | Colorectal | STAGE II | 1.0283 | 100.038914 |
| | | | | | Adenocarcinoma | | | |
| Q553L | c.1658A > T | PD2 | 2 | COADREAD | Colorectal | STAGE II | 1.02817 | 100.026267 |
| | | | | | Adenocarcinoma | | | |
| D1277A | c.3830A > C | VSD4 | 4 | COADREAD | Colorectal | STAGE II | 1.02814 | 100.023349 |
| | | | | | Adenocarcinoma | | | |
| T1320M | c.3959C > T | S4-S5 | 3 | COADREAD | Colorectal | STAGE II | 1.02802 | 100.011574 |
| | | LINKER | | | Adenocarcinoma | | | |
| A1217T | c.3649G > A | VSD4 | 4 | STAD | Tubular Stomach | STAGE II | 1.02796 | 100.005837 |
| | | | | | Adenocarcinoma | | | |
| S980L | c.2705C > T | VSD3 | 3 | COADREAD | Colon | STAGE II | 1.02791 | 100.000973 |
| | | | | | Adenocarcinoma | | | |
| A424D | c.1271C > A | VSD2 | 2 | ESCC | Esophageal | STAGE II | 1.02838 | 100.046697 |
| | | | | | Squamous Cell | | | |
| | | | | | Carcinoma | | | |
| D211Y | c.631G > T | ECL | ECL | COADREAD | Colon | STAGE II | 1.02826 | 100.035023 |
| | | | | | Adenocarcinoma | | | |
| E1518K | c.4552G > A | CTD | CTD | COADREAD | Colorectal | STAGE II | 1.02712 | 99.9241171 |
| | | | | | Adenocarcinoma | | | |
| E128G | c.383A > G | VSD1 | 1 | COADREAD | Colorectal | STAGE II | 1.02762 | 99.97276 |
| | | | | | Adenocarcinoma | | | |
| R1273I | c.3818G > T | VSD4 | 4 | COADREAD | Colorectal | STAGE II | 1.0276 | 99.9708143 |
| | | | | | Adenocarcinoma | | | |
| E432D | c.1296A > C | VSD2 | 2 | COADREAD | Colorectal | STAGE II | 1.02752 | 99.9630314 |
| | | | | | Adenocarcinoma | | | |
| D1277N | c.3829G > A | VSD4 | 4 | STAD | Diffuse Type | STAGE II | 1.02748 | 99.95914 |
| | | | | | Stomach | | | |
| | | | | | Adenocarcinoma | | | |
| S121C | c.362C > G | VSD1 | 1 | COADREAD | Colorectal | STAGE II | 1.0272 | 99.9319 |
| | | | | | Adenocarcinoma | | | |
| I51S | c.152T > G | VSD1 | 1 | COADREAD | Mucinous | STAGE II | 1.0271 | 99.9221714 |
| | | | | | Adenocarcinoma | | | |
| | | | | | of the Colon | | | |
| | | | | | and Rectum | | | |
| R989Q | c.2956G > A | VSD3 | 3 | COADREAD | Colorectal | STAGE II | 1.02671 | 99.88423 |
| | | | | | Adenocarcinoma | | | |
| I322F | c.964A > T | PD1 | 1 | COADREAD | Colorectal | STAGE II | 1.02604 | 99.8190486 |
| | | | | | Adenocarcinoma | | | |
| D952N | c.2854G > A | VSD3 | 3 | COADREAD | Colorectal | STAGE II | 1.02603 | 99.8180757 |
| | | | | | Adenocarcinoma | | | |
| K1069N | c.3207G > C | ECL | ECL | HNSCC | Head and Neck | STAGE III | 1.02845 | 100.053507 |
| | | | | | Squamous Cell | | | |
| | | | | | Carcinoma | | | |
| D1099N | c.3295G > A | ECL | ECL | COADREAD | Colorectal | STAGE III | 1.02828 | 100.036969 |
| | | | | | Adenocarcinoma | | | |
| A310T | c.928G > A | PD1 | 1 | BUC | Bladder Urothelial | STAGE III | 1.02808 | 100.017511 |
| | | | | | Carcinoma | | | |
| F1311L | c.3933T > G | VSD4 | 4 | STAD | Intestinal | STAGE III | 1.02762 | 99.97276 |
| | | | | | Type Stomach | | | |
| | | | | | Adenocarcinoma | | | |
| G1303D | c.3908G > A | VSD4 | 4 | COADREAD | Colorectal | STAGE III | 1.02741 | 99.95233 |
| | | | | | Adenocarcinoma | | | |
| R152O | c.455G > A | VSD1 | 1 | STAD | Mucinous Stomach | STAGE III | 1.02656 | 99.8696371 |
| | | | | | Adenocarcinoma | | | |
| E62K | c.184G > A | VSD1 | 1 | UEC | Uterine | STAGE III | 1.02566 | 99.78208 |
| | | | | | Endometrioid | | | |
| | | | | | Carcinoma | | | |
| G1526S | c.4576G > A | CTD | CTD | STAD | Mucinous Stomach | STAGE III | 0.99777 | 97.068781 |
| | | | | | Adenocarcinoma | | | |
| S1068A | c.3202T > G | ECL | ECL | EAD | Esophageal | STAGE III | 0.99612 | 96.9082596 |
| | | | | | Adenocarcinoma | | | |
| V1229F | c.3685G > T | VSD4 | 4 | COADREAD | Colorectal | STAGE III | 0.76993 | 74.9032007 |
| | | | | | Adenocarcinoma | | | |
| L588M | c.1762C > A | PD2 | 2 | STAD | Gastric | STAGE III | 0.44201 | 43.0790933 |
| | | | | | Adenocarcinoma | | | |
| O279H | c.837G > T | PD1 | 1 | COADREAD | Colorectal | STAGE III | 1.01837 | 99.072867 |
| | | | | | Adenocarcinoma | | | |
| E257G | c.770A > G | ECL | ECL | EAD | Esophageal | STAGE III | 0.99759 | 97.0512696 |
| | | | | | Adenocarcinoma | | | |
| E1458K | c.4372G > A | PD4 | 4 | STAD | Stomach | STAGE III | 0.8436 | 82.0702403 |
| | | | | | Adenocarcinoma | | | |
| A1044V | c.3131C > T | ECL | ECL | STAD | Tubular Stomach | STAGE III | 0.99838 | 97.1281253 |
| | | | | | Adenocarcinoma | | | |
| Y1349H | c.4045T > C | VSD4 | 4 | EAC | Esophageal | STAGE III | 0.99825 | 97.1154782 |
| | | | | | Adenocarcinoma | | | |
| F300S | c.899T > C | PD1 | 1 | STAD | Stomach | STAGE III | 0.99755 | 97.0473782 |
| | | | | | Adenocarcinoma | | | |
| E454K | c.1360G > A | VSD2 | 2 | STAD | Tubular Stomach | STAGE III | 0.99312 | 96.6164024 |
| | | | | | Adenocarcinoma | | | |
| C970Y | c.734G > T | VSD3 | 3 | COADREAD | Colorectal | STAGE III | 0.99157 | 96.4656095 |
| | | | | | Adenocarcinoma | | | |
| V510F | c.1528G > T | VSD2 | 2 | ESCC | Esophageal | STAGE III | 0.9909 | 96.4004281 |
| | | | | | Squamous Cell | | | |
| | | | | | Carcinoma | | | |
| R1495O | c.4484G > A | CTD | CTD | COADREAD | Colorectal | STAGE III | 0.9693 | 94.2990563 |
| | | | | | Adenocarcinoma | | | |
| R1384Q | c.4151G > A | PD4 | 4 | SCC | Cutaneous | STAGE III | 0.88754 | 86.3449752 |
| | | | | | Squamous Cell | | | |
| | | | | | Carcinoma | | | |
| V53D | c.158T > A | VSD1 | 1 | COADREAD | Colorectal | STAGE III | 0.83607 | 81.3376788 |
| | | | | | Adenocarcinoma | | | |
| M520V | c.1558A > G | VSD2 | 2 | ESCC | Esophageal | STAGE III | 0.83543 | 81.2754159 |
| | | | | | Squamous Cell | | | |
| | | | | | Carcinoma | | | |
| T272I | c.815C > T | PD1 | 1 | COADREAD | Colon | STAGE III | 0.78829 | 76.6893667 |
| | | | | | Adenocarcinoma | | | |
| A1091V | c.3272C > T | ECL | ECL | COADREAD | Colorectal | STAGE III | 0.76732 | 74.549285 |
| | | | | | Adenocarcinoma | | | |
| C1348W | c.4044T > G | VSD4 | 4 | EAC | Esophageal | STAGE III | 0.73727 | 71.7258488 |
| | | | | | Adenocarcinoma | | | |
| F1018I | c.3052T > A | VSD3 | 3 | EAC | Esophageal | STAGE III | 1.02841 | 100.049616 |
| | | | | | Adenocarcinoma | | | |
| M986I | c.2958G > T | VSD3 | 3 | COADREAD | Colorectal | STAGE III | 1.02883 | 100.090476 |
| | | | | | Adenocarcinoma | | | |
| I1017N | c.3050T > A | VSD3 | 3 | COADREAD | Colorectal | STAGE III | 1.02871 | 100.078801 |
| | | | | | Adenocarcinoma | | | |
| D1171N | c.3511G > A | DIII-IV | 3 | COADREAD | Colorectal | STAGE III | 1.02865 | 100.072964 |
| | | LINKER | | | Adenocarcinoma | | | |
| O238H | c.714G > C | ECL | ECL | EAD | Esophageal | STAGE III | 1.02858 | 100.066154 |
| | | | | | Adenocarcinoma | | | |
| R1496C | c.4492C > T | CTD | CTD | MESO | Pleural | STAGE III | 1.02849 | 100.057399 |
| | | | | | Mesothelioma | | | |
| | | | | | Epithelioid Type | | | |
| G193E | c.578G > A | VSD1 | 1 | SCC | Cutaneous | STAGE III | 1.02839 | 100.04767 |
| | | | | | Squamous Cell | | | |
| | | | | | Carcinoma | | | |
| M1244T | c.3731T > C | VSD4 | 4 | ESCC | Esophageal | STAGE III | 1.02835 | 100.043779 |
| | | | | | Squamous Cell | | | |
| | | | | | Carcinoma | | | |
| P467R | c.1400C > G | VSD2 | 2 | ESCC | Esophageal | STAGE III | 1.0283 | 100.038914 |
| | | | | | Squamous Cell | | | |
| | | | | | Carcinoma | | | |
| K1491T | c.4472A > C | CTD | CTD | STAD | Diffuse Type | STAGE III | 1.02817 | 100.026267 |
| | | | | | Stomach | | | |
| | | | | | Adenocarcinoma | | | |
| R1127C | c.3379C > T | PD3 | 3 | COADREAD | Colorectal | STAGE III | 1.02811 | 100.02043 |
| | | | | | Adenocarcinoma | | | |
| N1070K | c.3210C > A | ECL | ECL | ESCC | Esophageal | STAGE III | 1.02787 | 99.9970814 |
| | | | | | Squamous Cell | | | |
| | | | | | Carcinoma | | | |
| L305V | c.913C > G | PD1 | 1 | EAC | Esophageal | STAGE III | 1.02656 | 99.8696371 |
| | | | | | Adenocarcinoma | | | |
| G954S | c.2860G > A | VSD3 | 3 | COADREAD | Rectal | STAGE III | 1.02782 | 99.9922171 |
| | | | | | Adenocarcinoma | | | |
| P908L | c.2723C > T | VSD3 | 3 | COADREAD | Colorectal | STAGE III | 1.02772 | 99.9824886 |
| | | | | | Adenocarcinoma | | | |
| L1548F | c.4644G > T | CTD | CTD | ESCC | Esophageal | STAGE III | 1.0274 | 99.9513571 |
| | | | | | Squamous Cell | | | |
| | | | | | Carcinoma | | | |
| A88T | c.262G > A | VSD1 | 1 | ESCC | Esophageal | STAGE III | 1.02732 | 99.9435743 |
| | | | | | Squamous Cell | | | |
| | | | | | Carcinoma | | | |
| V120A | c.359T > C | VSD1 | 1 | STAD | Diffuse Type | STAGE III | 1.02725 | 99.9367643 |
| | | | | | Stomach | | | |
| | | | | | Adenocarcinoma | | | |
| T57R | c.178C > G | VSD1 | 1 | COADREAD | Colorectal | STAGE III | 1.02718 | 99.9299543 |
| | | | | | Adenocarcinoma | | | |
| V1285I | c.3853G > A | VSD4 | 4 | RCCC | Rectal Clear | STAGE III | 1.02706 | 99.91828 |
| | | | | | Cell Carcinoma | | | |
| F154S | c.461T > C | VSD1 | 1 | STAD | Diffuse Type | STAGE III | 1.0261 | 99.8248857 |
| | | | | | Stomach | | | |
| | | | | | Adenocarcinoma | | | |
| Q555R | c.1663G > A | PD2 | 2 | COADREAD | Colorectal | STAGE III | 1.02518 | 99.7353828 |
| | | | | | Adenocarcinoma | | | |
| R159Q | c.476G > A | VSD1 | 1 | UEC | Uterine | STAGE III | 1.02025 | 99.2557642 |
| | | | | | Endometrioid | | | |
| | | | | | Carcinoma | | | |
| R143W | c.427C > T | VSD1 | 1 | COADREAD | Colorectal | STAGE IV | 1.02682 | 99.8949314 |
| | | | | | Adenocarcinoma | | | |
| L1461F | c.4381C > T | CTD | CTD | STAD | Stomach | STAGE IV | 1.02577 | 99.7927814 |
| | | | | | Adenocarcinoma | | | |
| R1540W | c.4618C > T | CTD | CTD | PRAD | Prostate | STAGE IV | 0.9972 | 97.0133281 |
| | | | | | Adenocarcinoma | | | |
| F540S | c.1619T > C | PD2 | 2 | COADREAD | Colorectal | STAGE IV | 0.8871 | 86.3021695 |
| | | | | | Adenocarcinoma | | | |
| T1281M | c.3842C > T | VSD4 | 4 | UEC | Uterine | STAGE IV | 0.99845 | 97.1349363 |
| | | | | | Endometrioid | | | |
| | | | | | Carcinoma | | | |
| E327K | c.979G > A | PD1 | 1 | Bladder | Bladder | STAGE IV | 0.99872 | 97.1213153 |
| | | | | Urothelial | Urothelial | | | |
| | | | | Cancer | Cancer | | | |
| E323K | c.967G > A | PD1 | 1 | Melanoma | Cutaneous | STAGE IV | 0.99831 | 97.1213153 |
| | | | | | Melanoma | | | |
| E1552K | c.4654G > A | CTD | CTD | Melanoma | Cutaneous | STAGE IV | 0.99785 | 97.0765639 |
| | | | | | Melanoma | | | |
| V1239A | c.3716T > C | VSD4 | 4 | STAD | Tubular Stomach | STAGE IV | 0.99784 | 97.075591 |
| | | | | | Adenocarcinoma | | | |
| R1495W | c.4483C > T | CTD | CTD | GBM | Glioblastoma | STAGE IV | 0.99562 | 96.9569024 |
| | | | | | Multiforme | | | |
| S174L | c.521C > T | VSD1 | 1 | COADREAD | Colorectal | STAGE IV | 0.99569 | 96.8664267 |
| | | | | | Adenocarcinoma | | | |
| M55I | c.165G > A | VSD1 | 1 | ESCC | Esophageal | STAGE IV | 0.99003 | 87.5600739 |
| | | | | | Squamous Cell | | | |
| | | | | | Carcinoma | | | |
| Y1300S | c.3899A > C | VSD4 | 4 | COADREAD | Rectal | STAGE IV | 0.88485 | 86.0832755 |
| | | | | | Adenocarcinoma | | | |
| R43H | c.128G > A | VSD1 | 1 | GBM | Glioblastoma | STAGE IV | 0.80355 | 78.1739469 |
| | | | | | Multiforme | | | |
| D416N | c.1246G > A | VSD2 | 2 | Melanoma | Melanoma | STAGE IV | 0.7356 | 71.5633817 |
| R855Q | c.2564G > A | S1NA- | 2 | COADREAD | Colorectal | STAGE IV | 0.66229 | 64.4313649 |
| | | S1NB | | | Adenocarcinoma | | | |
| V511A | c.1532T > C | VSD2 | 2 | COADREAD | Colorectal | STAGE IV | 1.02817 | 100.026267 |
| | | | | | Adenocarcinoma | | | |
| D1527N | c.4579G > A | CTD | CTD | Melanoma | Melanoma | STAGE IV | 1.02772 | 99.9824886 |
| R995C | c.2983C > T | VSD3 | 3 | COADREAD | Colorectal | STAGE IV | 1.02857 | 100.065181 |
| | | | | | Adenocarcinoma | | | |
| R146Q | c.437G > A | VSD1 | 1 | SSC | squamous cell | STAGE IV | 1.02803 | 100.012647 |
| | | | | | carcinoma | | | |
| G1013S | c.3037G > A | S4-S5 | 3 | COADREAD | Colorectal | STAGE IV | 1.02783 | 99.99319 |
| | | LINKER | | | Adenocarcinoma | | | |
| R1193C | c.3577C > T | DIII-IV | 3 | Melanoma | Cutaneous | STAGE IV | 1.02701 | 99.9134157 |
| | | LINKER | | | Melanoma | | | |
| R143Q | c.428G > A | VSD1 | 1 | STAD | Stomach | STAGE IV | 1.02664 | 99.87742 |
| | | | | | Adenocarcinoma | | | |
| P65S | c.193C > T | VSD1 | 1 | STAD | Stomach | STAGE IV | 1.02602 | 99.8171028 |
| | | | | | Adenocarcinoma | | | |
| V1490I | c.4458G > A | CTD | CTD | COADREAD | Colon | STAGE IV | 1.02212 | 99.4376885 |
| | | | | | Adenocarcinoma | | | |
| T539M | c.1616C > T | PD2 | 2 | IHC | Intrahepatic | STAGE IV | 1.02022 | 99.2526456 |
| | | | | | Cholangio- | | | |
| | | | | | carcinoma | | | |
| WT | | | | | | | 1.0279 | 100 |

| | predicted | met risk | Gate radius | | | met risk | |
|---|---|---|---|---|---|---|---|
| | effect on | based on | (WT Gate | % of WT | predicted | based on | overall |
| | selective | filter | radius = | gate | effect on | gate | met risk |
| Mutation | filter | radius | 0.61617 | radius | gate | radius | score |
| R1481S | LOF | medium | 0.21756 | 35.3084376 | LOF | high | high |
| G1316V | LOF | medium | −0.27819 | −45.148255 | LOF | high | high |
| A401V | LOF | high | 0.24188 | 39.2554003 | LOF | high | high |
| L253H | LOF | high | 0.77898 | 126.422903 | GOF | unknown | high |
| N1475K | LOF | high | 0.77938 | 126.48782 | GOF | unknown | high |
| V273I | LOF | high | 0.78049 | 126.667965 | GOF | unknown | high |
| D134Y | LOF | high | 0.77086 | 125.105085 | GOF | unknown | high |
| S1264L | no effect | low | 0.77838 | 126.325527 | GOF | unknown | low |
| K1230N | no effect | low | 0.77095 | 125.119691 | GOF | unknown | low |
| V50I | no effect | low | 0.73344 | 119.032085 | GOF | unknown | low |
| M1425L | no effect | low | 0.7794 | 126.491066 | GOF | unknown | low |
| A223D | no effect | low | 0.77964 | 126.530016 | GOF | unknown | low |
| V1503A | no effect | low | 0.78005 | 126.596556 | GOF | unknown | low |
| P66L | no effect | low | 0.60949 | 98.9158836 | LOF | medium | medium |
| H39P | no effect | low | 0.61148 | 99.2388464 | LOF | medium | medium |
| F1250S | no effect | low | 0.60627 | 98.3933006 | LOF | medium | medium |
| F389L | LOF | medium | 0.72951 | 118.394274 | GOF | unknown | medium |
| L1442P | LOF | medium | 0.67098 | 108.895272 | GOF | unknown | medium |
| A401T | LOF | medium | 0.77769 | 126.213545 | GOF | unknown | medium |
| K498T | LOF | medium | 0.77968 | 126.536508 | GOF | unknown | medium |
| V1329I | LOF | medium | 0.7801 | 126.604671 | GOF | unknown | medium |
| S52P | LOF | medium | 0.66961 | 108.672931 | GOF | unknown | medium |
| L942S | LOF | medium | 0.7789 | 126.409919 | GOF | unknown | medium |
| R1193H | no effect | low | 0.1366 | 22.1692055 | LOF | high | high |
| T1165M | LOF | medium | 0.40517 | 65.7562037 | LOF | high | high |
| R166I | LOF | medium | 0.16694 | 27.0931723 | LOF | high | high |
| K452E | LOF | medium | 0.08592 | 13.9442037 | LOF | high | high |
| R1500S | LOF | high | 0.50323 | 81.6706428 | LOF | high | high |
| F1427L | LOF | high | 0.61688 | 100.115228 | no effect | low | high |
| H876R | LOF | high | 0.6169 | 100.118474 | no effect | low | high |
| R295H | LOF | high | 0.61675 | 100.09413 | no effect | low | high |
| A1421V | LOF | high | 0.61682 | 100.10549 | no effect | low | high |
| V1386I | LOF | high | 0.61709 | 100.149309 | no effect | low | high |
| L564V | LOF | high | 0.77991 | 126.573835 | GOF | unknown | high |
| V1007A | LOF | high | 0.67035 | 108.793028 | GOF | unknown | high |
| L1553P | LOF | high | 0.7717 | 125.241411 | GOF | unknown | high |
| R995H | LOF | high | 0.76687 | 124.457535 | GOF | unknown | high |
| R382W | LOF | high | 0.77968 | 126.536508 | GOF | unknown | high |
| S902F | LOF | high | 0.76882 | 124.774007 | GOF | unknown | high |
| S384F | LOF | high | 0.77196 | 125.286853 | GOF | unknown | high |
| V385I | LOF | high | 0.70717 | 114.768652 | GOF | unknown | high |
| R1556G | LOF | high | 0.66976 | 108.697275 | GOF | unknown | high |
| L999V | LOF | high | 0.73451 | 119.205739 | GOF | unknown | high |
| R1174I | LOF | high | 0.77924 | 126.465099 | GOF | unknown | high |
| V1542M | LOF | high | 0.77956 | 126.517033 | GOF | unknown | high |
| V320A | LOF | high | 0.77854 | 126.351494 | GOF | unknown | high |
| S403G | no effect | low | 0.78001 | 126.590064 | GOF | unknown | low |
| L222S | no effect | low | 0.70015 | 126.612785 | GOF | unknown | low |
| V400M | no effect | low | 0.75609 | 122.708019 | GOF | unknown | low |
| R43C | no effect | low | 0.74609 | 121.08509 | GOF | unknown | low |
| Q553L | no effect | low | 0.78033 | 126.641998 | GOF | unknown | low |
| D1277A | no effect | low | 0.73372 | 119.077527 | GOF | unknown | low |
| T1320M | no effect | low | 0.77847 | 126.340133 | GOF | unknown | low |
| A1217T | no effect | low | 0.77958 | 126.536508 | GOF | unknown | low |
| S980L | no effect | low | 0.77917 | 126.453738 | GOF | unknown | low |
| A424D | no effect | low | 0.60924 | 98.8753104 | LOF | medium | medium |
| D211Y | no effect | low | 0.60725 | 98.5523476 | LOF | medium | medium |
| E1518K | LOF | medium | 0.60905 | 98.8444747 | LOF | medium | medium |
| E128G | LOF | medium | 0.77891 | 126.411542 | GOF | unknown | medium |
| R1273I | LOF | medium | 0.78017 | 126.616031 | GOF | unknown | medium |
| E432D | LOF | medium | 0.77901 | 126.427772 | GOF | unknown | medium |
| D1277N | LOF | medium | 0.77956 | 126.517033 | GOF | unknown | medium |
| S121C | LOF | medium | 0.77984 | 126.562475 | GOF | unknown | medium |
| I51S | LOF | medium | 0.77969 | 126.538131 | GOF | unknown | medium |
| R989Q | LOF | medium | 0.77855 | 126.353117 | GOF | unknown | medium |
| I322F | LOF | medium | 0.92196 | 149.627538 | GOF | unknown | medium |
| D952N | LOF | medium | 0.73512 | 119.304737 | GOF | unknown | medium |
| K1069N | no effect | low | 0.07649 | 12.4137819 | LOF | high | high |
| D1099N | no effect | low | 0.4456 | 72.3177045 | LOF | high | high |
| A310T | no effect | low | 0.0949 | 15.4015937 | LOF | high | high |
| F1311L | LOF | medium | 0.29577 | 48.0013633 | LOF | high | high |
| G1303D | LOF | medium | 0.55616 | 90.2608047 | LOF | high | high |
| R152O | LOF | medium | 0.40526 | 65.77081 | LOF | high | high |
| E62K | LOF | medium | 0.23854 | 38.7133421 | LOF | high | high |
| G1526S | LOF | high | 0.05238 | 8.50090073 | LOF | high | high |
| S1068A | LOF | high | 0.60169 | 97.6499992 | LOF | high | high |
| V1229F | LOF | high | 0.03539 | 5.7435448 | LOF | high | high |
| L588M | LOF | high | 0.44281 | 71.8649074 | LOF | high | high |
| O279H | LOF | high | 0.61657 | 100.064917 | no effect | low | high |
| E257G | LOF | high | 0.60959 | 98.9321129 | LOF | medium | high |
| E1458K | LOF | high | 0.61609 | 99.9870165 | no effect | medium | high |
| A1044V | LOF | high | 0.77978 | 126.552737 | GOF | unknown | high |
| Y1349H | LOF | high | 0.77955 | 126.51541 | GOF | unknown | high |
| F300S | LOF | high | 0.77947 | 126.502426 | GOF | unknown | high |
| E454K | LOF | high | 0.76548 | 124.231949 | GOF | unknown | high |
| C970Y | LOF | high | 0.77955 | 126.51541 | GOF | unknown | high |
| V510F | LOF | high | 0.73544 | 119.356671 | GOF | unknown | high |
| R1495O | LOF | high | 0.77958 | 126.520279 | GOF | unknown | high |
| R1384Q | LOF | high | 0.77791 | 126.249249 | GOF | unknown | high |
| V53D | LOF | high | 0.77892 | 126.413165 | GOF | unknown | high |
| M520V | LOF | high | 0.77898 | 126.422903 | GOF | unknown | high |
| T272I | LOF | high | 0.77969 | 126.538131 | GOF | unknown | high |
| A1091V | LOF | high | 0.76716 | 124.504601 | GOF | unknown | high |
| C1348W | LOF | high | 0.73727 | 119.653667 | GOF | unknown | high |
| F1018I | no effect | low | 0.51675 | 100.09413 | no effect | low | low |
| M986I | no effect | low | 0.77932 | 126.478082 | GOF | unknown | low |
| I1017N | no effect | low | 0.68484 | 111.144652 | GOF | unknown | low |
| D1171N | no effect | low | 0.64802 | 105.169028 | GOF | unknown | low |
| O238H | no effect | low | 0.77899 | 126.424526 | GOF | unknown | low |
| R1496C | no effect | low | 0.7791 | 126.442378 | GOF | unknown | low |
| G193E | no effect | low | 0.77223 | 125.327426 | GOF | unknown | low |
| M1244T | no effect | low | 0.77995 | 126.580327 | GOF | unknown | low |
| P467R | no effect | low | 0.66879 | 108.539851 | GOF | unknown | low |
| K1491T | no effect | low | 0.66972 | 108.690783 | GOF | unknown | low |
| R1127C | no effect | low | 0.78029 | 126.635506 | GOF | unknown | low |
| N1070K | no effect | medium | 0.61129 | 99.2080108 | LOF | medium | medium |
| L305V | LOF | medium | 0.61517 | 99.8377071 | no effect | medium | medium |
| G954S | no effect | medium | 0.77181 | 125.259263 | GOF | unknown | medium |
| P908L | no effect | medium | 0.65998 | 107.110051 | GOF | unknown | medium |
| L1548F | LOF | medium | 0.6709 | 108.882289 | GOF | unknown | medium |
| A88T | LOF | medium | 0.66963 | 108.676177 | GOF | unknown | medium |
| V120A | LOF | medium | 0.77772 | 126.218414 | GOF | unknown | medium |
| T57R | LOF | medium | 0.67032 | 108.788159 | GOF | unknown | medium |
| V1285I | LOF | medium | 0.77908 | 126.439132 | GOF | unknown | medium |
| F154S | LOF | medium | 0.77913 | 126.447247 | GOF | unknown | medium |
| Q555R | LOF | medium | 0.77932 | 126.478082 | GOF | unknown | medium |
| R159Q | LOF | medium | 0.78042 | 126.656605 | GOF | unknown | medium |
| R143W | LOF | medium | 0.09045 | 14.6793904 | LOF | high | high |
| L1461F | LOF | medium | 0.16899 | 27.4258727 | LOF | high | high |
| R1540W | LOF | high | 0.60909 | 98.8509665 | LOF | medium | high |
| F540S | LOF | high | 0.61599 | 99.9707873 | no effect | medium | high |
| T1281M | LOF | high | 0.67055 | 108.825487 | GOF | unknown | high |
| E327K | LOF | high | 0.78038 | 126.650113 | GOF | unknown | high |
| E323K | LOF | high | 0.77981 | 126.557606 | GOF | unknown | high |
| E1552K | LOF | high | 0.66874 | 108.531736 | GOF | unknown | high |
| V1239A | LOF | high | 0.77972 | 126.543 | GOF | unknown | high |
| R1495W | LOF | high | 0.78011 | 126.606294 | GOF | unknown | high |
| S174L | LOF | high | 0.77212 | 125.309574 | GOF | unknown | high |
| M55I | LOF | high | 0.7708 | 125.095347 | GOF | unknown | high |
| Y1300S | LOF | high | 0.77981 | 126.557606 | GOF | unknown | high |
| R43H | LOF | high | 0.73429 | 119.170034 | GOF | unknown | high |
| D416N | LOF | high | 0.67056 | 108.827109 | GOF | unknown | high |
| R855Q | LOF | high | 0.66229 | 107.484947 | GOF | unknown | high |
| V511A | no effect | low | 0.77934 | 126.481328 | GOF | unknown | low |
| D1527N | LOF | medium | 0.61708 | 100.147687 | no effect | low | medium |
| R995C | no effect | low | 0.60933 | 98.8899167 | LOF | medium | medium |
| R146Q | no effect | low | 0.6093 | 98.885048 | LOF | medium | medium |
| G1013S | no effect | medium | 0.74182 | 120.3921 | GOF | unknown | medium |
| R1193C | LOF | medium | 0.73679 | 119.575766 | GOF | unknown | medium |
| R143Q | LOF | medium | 0.73393 | 119.111609 | GOF | unknown | medium |
| P65S | LOF | medium | 0.64825 | 105.206355 | GOF | unknown | medium |
| V1490I | LOF | medium | 0.6695 | 108.655079 | GOF | unknown | medium |
| T539M | LOF | medium | 0.74119 | 120.289855 | GOF | unknown | medium |
| WT | no effect | low | 0.61617 | 100 | no effect | low | low |

Examples 6 to 10 relate to inventor's publication Rahrmann et al. The NALCN channel regulates metastasis and nonmalignant cell dissemination. Nature Genetics, doi.org/10.1038/s41588-022-01182-0, 2022. Extended data is available at https://doi.org/10.1038/s41588-022-01182-0. Supplementary information is available at https://doi.org/10.1038/s41588-022-01182-0.

### Example 6—NALCN Loss-of-Function in Cancer

In a related study to examples 1 to 5, it was demonstrated that the NALCN channel regulates metastasis and nonmalignant cell dissemination.

To determine how nonsynonymous mutations might affect NALCN function in cancer, we used HOLE analysis21 to predict their impact on the ion channel pore radius of NALCN embedded and relaxed within a 575-lipid 1-palmitoyl-2-oleoyl-sn-glycero-3-phosphocholine bilayer in silico12,22,23. This model correctly predicted opening of the NALCN channel by 22 mutations known to confer gain-of-functioni12, and closure of the channel by two mutations that cause loss-of-function11 (Rahrmann et al 2022—Supplementary Table 3 (reproduced below as Table 6)).

TABLE 6
(Supplementary Table 3 from Rahrmann et al 2022) In silico modeling
of electrophysiologically validated NALCN mutations

| | | | | | | NALCN | predicted |
|---|---|---|---|---|---|---|---|
| | | | electrophys- | Predicted | wild-type | gate | effect on |
| | | | iological | gate | gate_radius | radius % of | gate |
| publication | Disease | Mutation | effect | radius (Å) | (Å) | wild-type | radius |
| Chua H. E. et al 2020 | Cancer | R146Q | LOF | 0.6093 | 0.61617 | 98.88505 | LOF |
| Chua H. E. et al 2020 | Cancer | R152Q | LOF | 0.40526 | 0.61617 | 65.77081 | LOF |
| Kschonsak M et al 2020 | CLIFAHDD syndrome | E327K | GOF | 0.78038 | 0.61617 | 126.6501 | GOF |
| Kschonsak M et al 2020 | CLIFAHDD syndrome | A319V | GOF | 0.7783 | 0.61617 | 126.3125 | GOF |
| Kschonsak M et al 2020 | CLIFAHDD syndrome | T1165P | GOF | 0.66982 | 0.61617 | 108.707 | GOF |
| Kschonsak M et al 2020 | CLIFAHDD syndrome | R1181Q | GOF | 0.66993 | 0.61617 | 108.7249 | GOF |
| Kschonsak M et al 2020 | CLIFAHDD syndrome | I1446M | GOF | 0.67011 | 0.61617 | 108.7541 | GOF |
| Kschonsak M et al 2020 | CLIFAHDD syndrome | V1006A | GOF | 0.67034 | 0.61617 | 108.7914 | GOF |
| Kschonsak M et al 2020 | CLIFAHDD syndrome | F317C | GOF | 0.67037 | 0.61617 | 108.7963 | GOF |
| Kschonsak M et al 2020 | CLIFAHDD syndrome | L590F | GOF | 0.68208 | 0.61617 | 110.6967 | GOF |
| Kschonsak M et al 2020 | CLIFAHDD syndrome | V595F | GOF | 0.71162 | 0.61617 | 115.4909 | GOF |
| Kschonsak M et al 2020 | CLIFAHDD syndrome | V1020F | GOF | 0.7156 | 0.61617 | 116.1368 | GOF |
| Kschonsak M et al 2020 | CLIFAHDD syndrome | S524N | GOF | 0.73188 | 0.61617 | 118.7789 | GOF |
| Kschonsak M et al 2020 | CLIFAHDD syndrome | Y578C | GOF | 0.73273 | 0.61617 | 118.9169 | GOF |
| Kschonsak M et al 2020 | CLIFAHDD syndrome | L509S | GOF | 0.7671 | 0.61617 | 124.4949 | GOF |
| Kschonsak M et al 2020 | CLIFAHDD syndrome | R1181G | GOF | 0.76738 | 0.61617 | 124.5403 | GOF |
| Kschonsak M et al 2020 | CLIFAHDD syndrome | L312V | GOF | 0.77721 | 0.61617 | 126.1356 | GOF |
| Kschonsak M et al 2020 | CLIFAHDD syndrome | V597I | GOF | 0.77902 | 0.61617 | 126.4294 | GOF |
| Kschonsak M et al 2020 | CLIFAHDD syndrome | Q177P | GOF | 0.77956 | 0.61617 | 126.517 | GOF |
| Kschonsak M et al 2020 | CLIFAHDD syndrome | I1017T | GOF | 0.77963 | 0.61617 | 126.5284 | GOF |
| Kschonsak M et al 2020 | CLIFAHDD syndrome | Y578S | GOF | 0.77968 | 0.61617 | 126.5365 | GOF |
| Kschonsak M et al 2020 | CLIFAHDD syndrome | Y582S | GOF | 0.77974 | 0.61617 | 126.5462 | GOF |
| Kschonsak M et al 2020 | CLIFAHDD syndrome | F512V | GOF | 0.77981 | 0.61617 | 126.5576 | GOF |
| Kschonsak M et al 2020 | CLIFAHDD syndrome | V313G | GOF | 0.77988 | 0.61617 | 126.569 | GOF |

Nonsynonymous, cancer-associated mutations were clustered within the pore turret and voltage-sensing domains that regulate NALCN channel opening11,12: 75% (n=147/196) of these mutations were predicted to close the channel (FIG. 1c,d and Rahrmann et al 2022—Supplementary Table 4). Mutations predicted to cause the greatest pore closure were enriched in the most advanced cancers (FIG. 1e). Furthermore, human GACs in which NALCN was mutated, upregulated genes associated with EMT24, metastasis and cell migration (Rahrmann et al 2022—Supplementary Tables 5 and 6).

As a first step to test whether Nalcn regulates cancer progression, we altered its function in P1^{KP}-GAC cells using genetic (Nalcn-short hairpin RNA and NALCN-complementary DNA lentiviral transduction) or chemical (gadolinium chloride; GdCl₃)13 approaches. Whole-cell voltage-clamp analysis of P1^{KP}-GAC cells showed a linear GdCl3-sensitive current to voltage steps in the ±80 mV range, as previously reported13. Decreasing Nalcn expression in P1KP-GAC cells eliminated the NALCN current, increased cell proliferation and conferred an EMT morphology and transcriptome on P1^{KP}-GAC orthotopic allografts (Rahrmann et al 2022—Supplementary Tables 7,8). Conversely, increased Nalcn expression increased the GdCl₃-sensitive current in P1^{KP}-GAC cells, decreased cell proliferation and conferred a hyperepithelialized morphology on allografts.

### Example 7—Loss of Nalcn Promotes Cancer Metastasis

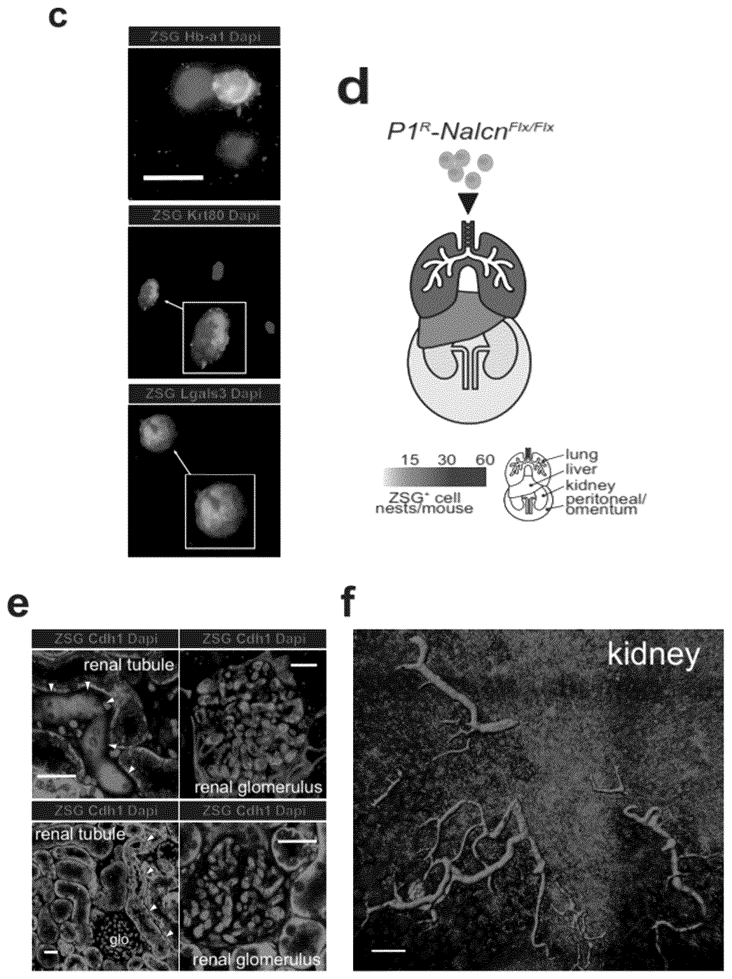

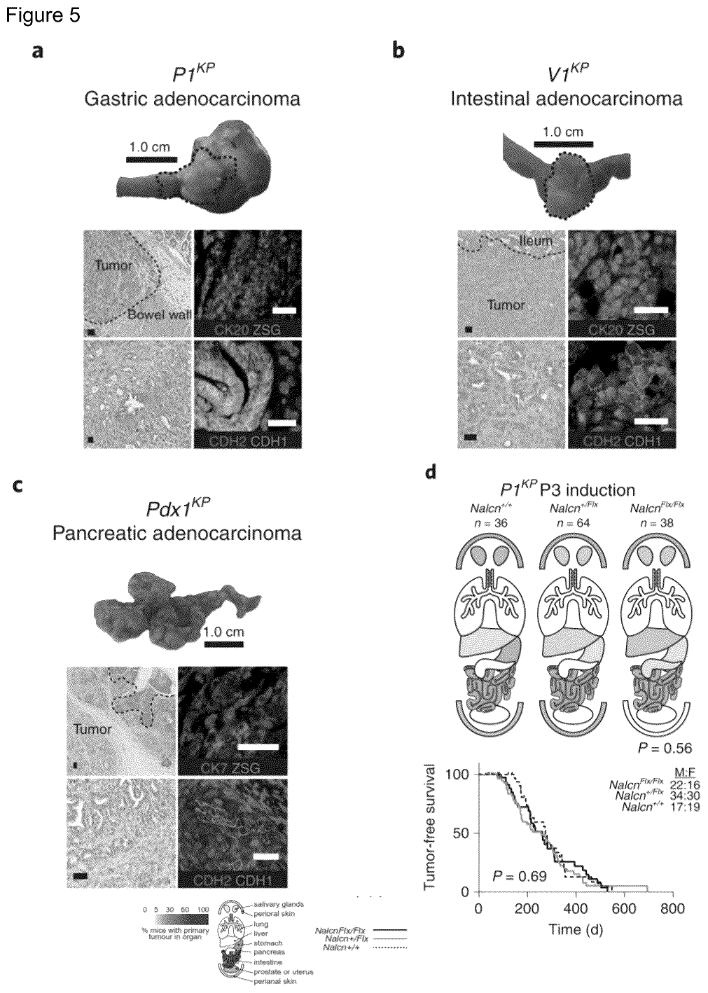

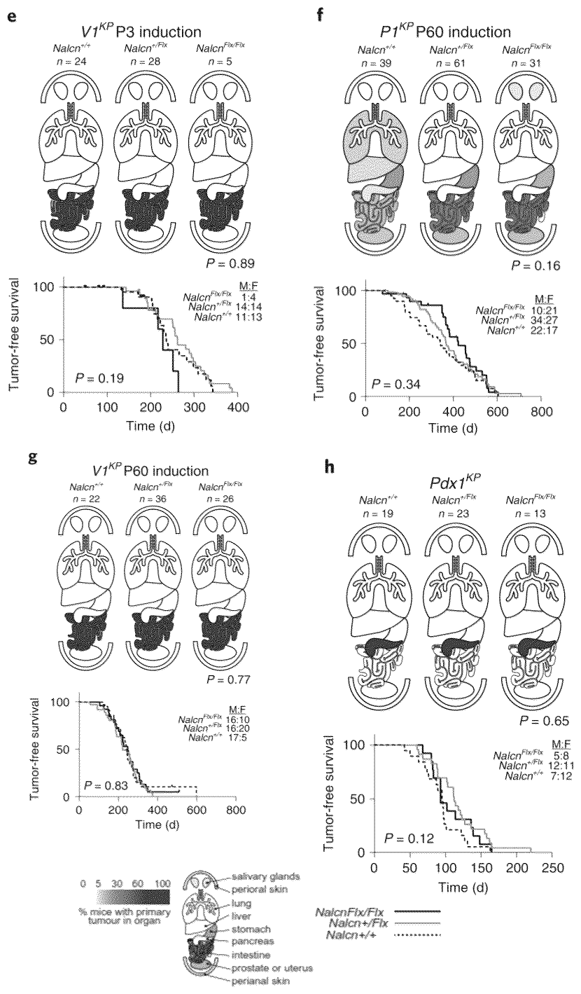

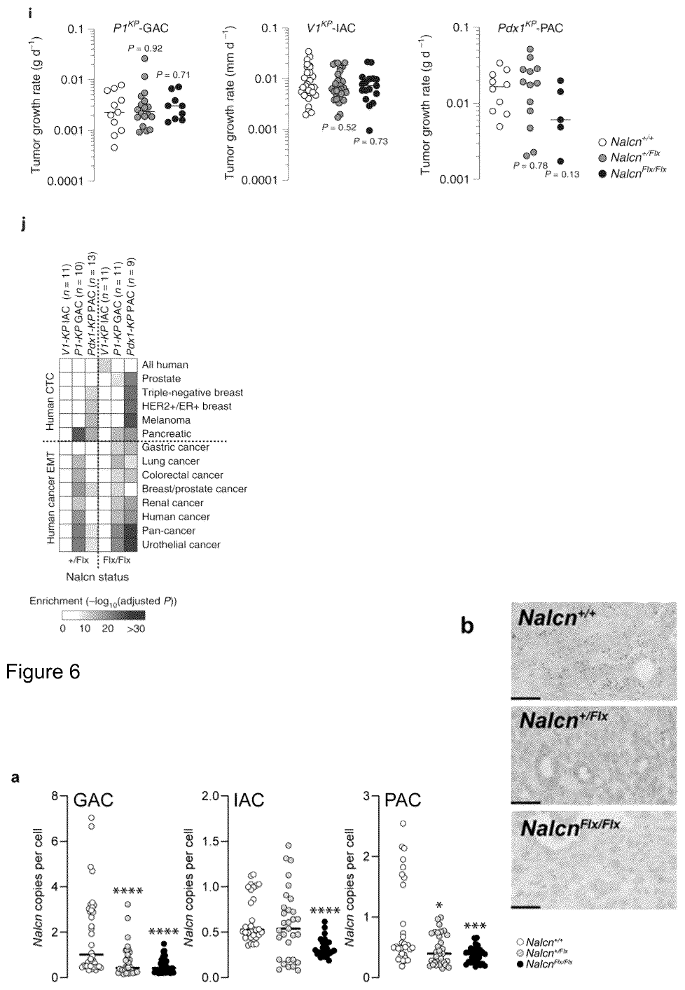

To study how Nalcn loss-of-function impacts cancer initiation and progression in intact tissues, we generated mice harboring a conditional Nalcn allele (Nalcn^{Flx}). These mice were bred with P1^{KP} Villin 1_Cre^{ERT2}; Kras^{G12D}; Trp53^{Flx/Flx} (V1^{KP}) or Pdx1-Cre; Kras^{G12D}; Trp53^{Flx/+} (Pdx1^{KP}) mice to produce equivalent numbers of male and female mice that were either Nalcn wild-type (Nalcn/), Nalcn/Flx or Nalcn^{Flx/Flx} (total n=551; Rahrmann et al 2022—Supplementary Table 9). All mice carried the Rosa26-ZsGreen (Rosa26^{ZSG}) lineage-tracing allele. Cancers in V1^{KP} and Pdx1^{KP} mice are restricted by Cre expression to the intestine25,26 and pancreas27,28, respectively. Prom1^{CreERT2/LacZ} is expressed by a variety of stem/progenitor cells and induces tumors of the small intestine, liver, lung, salivary glands, prostate, uterus, skin and stomach in P1^{KP} mice15,29. Because tissues can display age-dependent susceptibility to transformation15 we activated Cre-recombination in P1^{KP} and V1^{KP} mice using tamoxifen at postnatal day 3 (P3) or P60. As expected, V1^{KP} (n=127/141) and Pdx1^{KP} (n=55/55) mice developed intestinal and pancreatic tumors, respectively, whereas P1^{KP} mice developed tumors in the stomach (n=49/269), small intestine (n=59/269) and other sites (n=108/269)15,26,28; 99% (n=2121214) of tumors in P1^{KP} mice occurred as single primary tumor (FIG. 5a-g and Supplementary Table 9). Detailed macro- and microscopic analysis of tumors revealed no significant impact of age of induction, sex and/or Nalcn status on tumor incidence, type, tumor-free survival, tumor growth rate, immune cell infiltration, proliferation or other key primary tumor characteristics (FIG. 5, FIG. 6a-c and Tables 13-15). However, the transcriptomes of P1^{KP}-GAC and Pdx1^{KP} pancreatic adenocarcinomas (Pdx1^{KP}-PACs) were enriched for genes associated with human CTCs and EMT (Fig.).

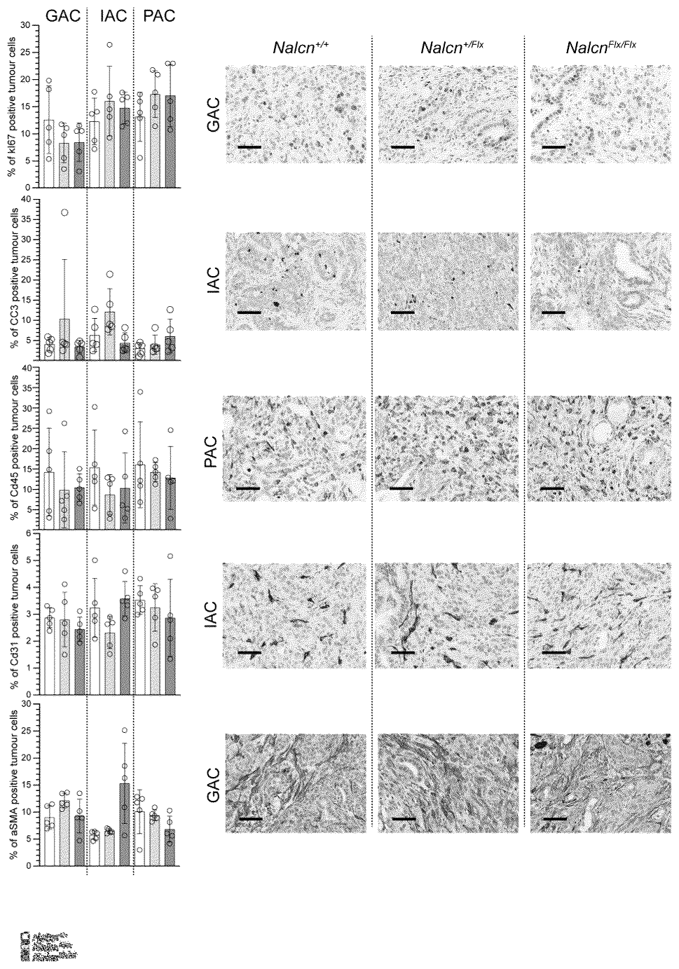

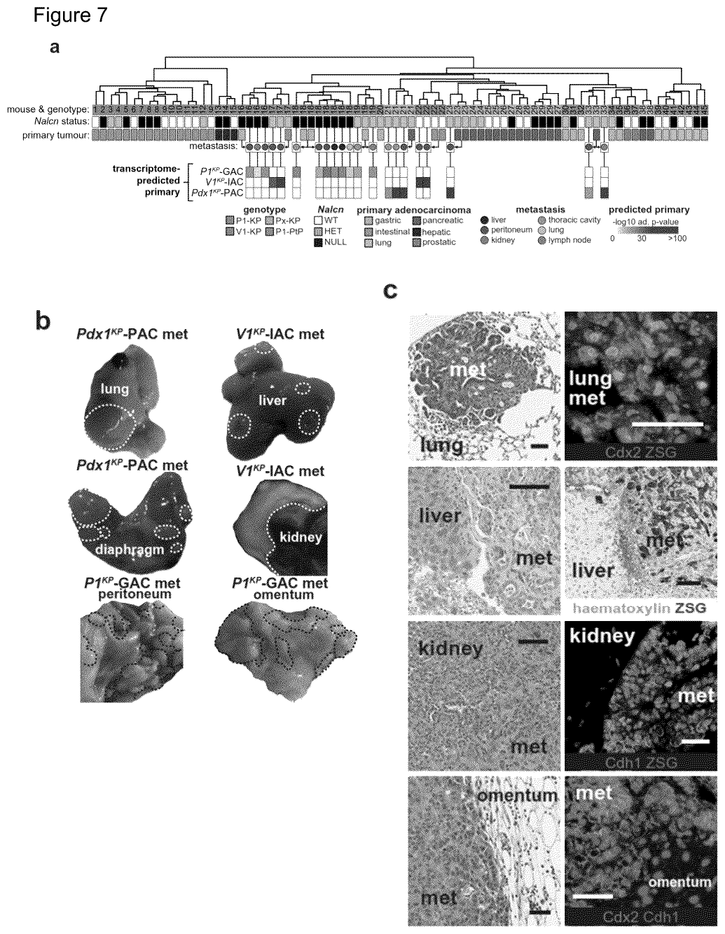

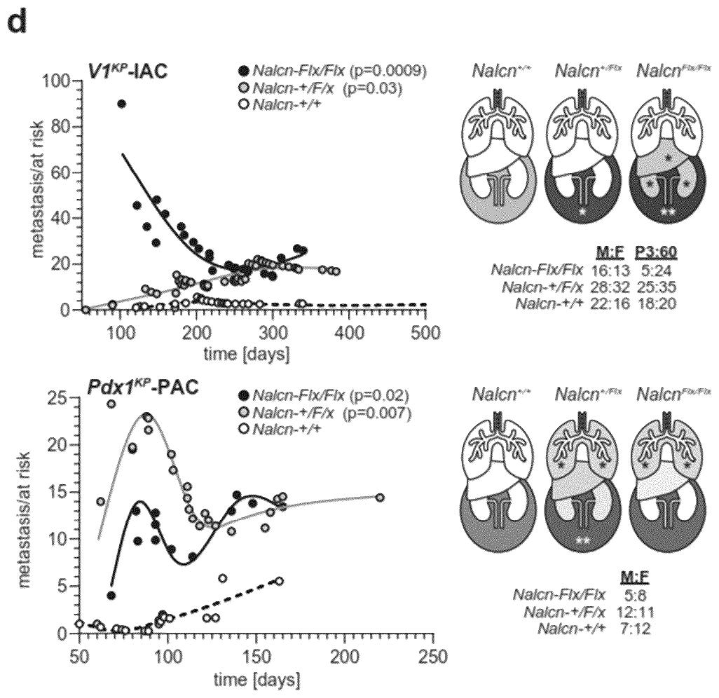

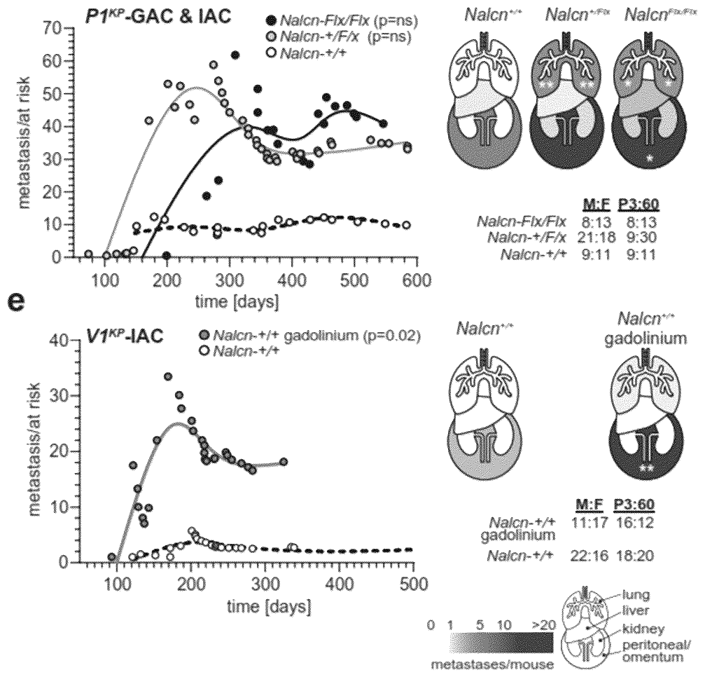

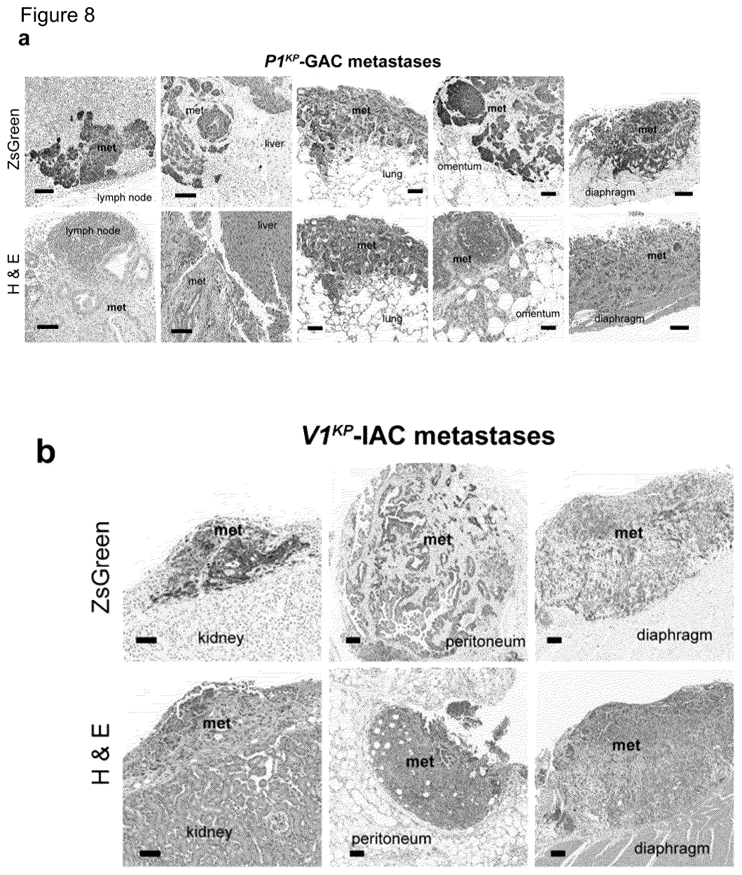

In keeping with these transcriptomic changes, deletion of Nalcn dramatically increased cancer metastasis in P1^{KP} V1^{KP} and Pdx1^{KP} mice (FIG. 7a-d and FIG. 8 and Rahrmann et al 2022—Supplementary Table 12). Metastatic and primary tumors were distinguished from one another by combined histology review, cosegregation of ‘matched’ primary and secondary tumor transcriptomes by unsupervised hierarchical clustering, and enrichment of histology-predicted primary tumor gene sets within metastatic tumor transcriptomes (FIG. 7a,c and FIG. 8). V1^{KP} intestinal adenocarcinomas (V1^{KP}-IACs, n=27 mice) and Pdx1^{KP}-PACs (n=19 mice) in Nalcn^{+/+} mice, produced 2.82±4.88 (mean±s.e.m.) and 5.53±4.02 metastases per mouse, respectively (FIG. 7d and Rahrmann et al 2022—Supplementary Tables 9 and 12). In stark contrast, these same tumors in V1^{KP}; Nalcn^{+/Flx} (n=51), V1^{KP}; Nalcn^{Flx/Flx} (n=26), Pdx1^{KP}; Nalcn^{+/Flx} (n23) and Pdx1^{KP}; Nalcn^{Flx/Flx} (n=13) mice, produced 16.82±5.69 (two-tailed Mann-Whitney U-test, P=0.03 relative to Nalcn/), 26.04±10.18 (P=0.0009), 15.04±3.62 (P=0.007) and 13.46±5.01 (P=0.02) metastases per mouse, respectively. Nalcn deletion from V1^{KP}-IACs increased metastasis in particular to the peritoneum, kidneys and liver: Nalcn deletion from Pdx1^{KP}-PACs increased metastasis to the peritoneum and lungs (FIG. 7d). Nalcn deletion also increased metastasis of IAC and GAC in P1^{KP} mice (n=80) from 11.60±3.45 metastases per P1^{KP}; Nalcn^{+/+} mouse to 42.21±11.23 metastases per P1^{KP}; Nalcn^{+/Flx} mouse and 40.24.0±15.51 metastases per P1^{KP}; Nalcn^{Flx/Flx} mouse (FIG. 7d and Rahrmann et al 2022—Supplementary Tables 9 and 12).

To further validate Nalcn loss-of-function as a driver of cancer metastasis, we treated additional cohorts of V1^{KP}; Nalcn^{+/+} (n=37), V1^{KP}; Nalcn^{+/Flx} (n=17) and V1^{KP}; Nalcn^{Flx/Flx} (n=8) mice with GdCl₃ (2 μg per kg (body weight) per week). IACs in GdCl3-treated V1^{KP}; Nalcn^{+/+} mice (n=28) produced 18.32±5.95 metastases per mouse compared with only 2.82±4.88 in controls (P=0.02; FIG. 7e and Rahrmann et al 2022—Supplementary Table 12). However, GdCl₃ did not increase metastasis in either V1^{KP}; Nalcn^{+/Flx} or V1^{KP}; Nalcn^{Flx/Flx} mice, confirming that the agent phenocopied the Nalcn-deletion metastatic phenotype, specifically.

### Example 8—NALCN Regulates CTCs

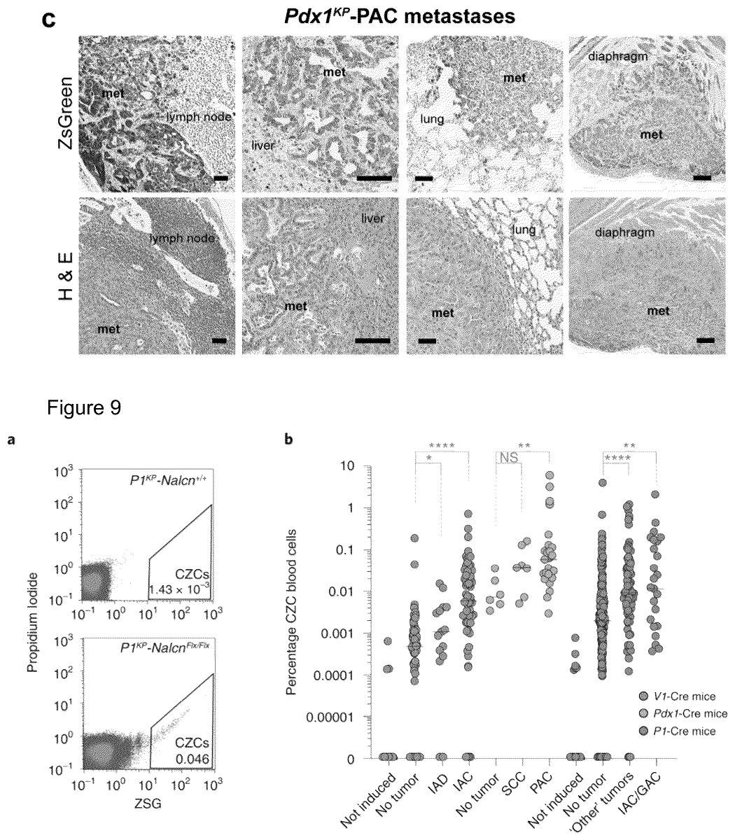

Because Nalcn deletion increased tumor metastasis and the expression by GACs, IACs and PACs of genes enriched in human CTC transcriptomes (FIG. 5j), we reasoned that Nalcn might regulate the release of CTCs from primary tumors: CTCs are shed from tumors into the blood as precursors of metastasis30. To test this, nucleated, GAC, IAC and PAC cells that had been genetically tagged by recombination of the Rosa26^{ZSG} lineage-tracing allele in the corresponding epithelium were isolated from whole blood and quantified using ZsGreen (ZSG)-fluorescence-activated cell sorting (FACS). Serial, peripheral blood samples taken from Prom1^{CreERT2/LacZ} (n=397), Villin-1^{CreERT2} (n=162) or Pdx1^{Cre} (n=40) mice that carried the Rosa26^{ZSG} allele and various combinations of oncogenic and Nalcn^{Flx} alleles were analyzed (Rahrmann et al 2022—Supplementary Table 13). An average (±s.e.m.) of 3.8×10^{3±0.9×10}³ circulating ZSG⁺ cells (CZCs) per ml of blood (0.066%±0.02% total cells) were isolated from all mice after an average of 254±9.1 d following Cre-recombination (FIG. 9a-c and Rahrmann et al 2022—Supplementary Table 13). Across all three Cre-lines, the number of CZCs was highly correlated with both the presence of a primary tumor (FIG. 9b) and the total number of metastases (multiple linear regression, T=10.43, P=0.000043; Rahrmann et al 2022—Supplementary Table 13), independent of mouse sex or age of induction. Nalcn deletion, or GdCl₃ treatment, significantly increased the level of CZCs in tumor-bearing P1^{KP}, V1^{KP} and Pdx1^{KP} mice (FIG. 9c). Because9neither Prom1^{CreERT2/LacZ}, Villin-1^{CreERT2} nor Pdx1^{Cre} recombine hematopoeitic cells in the bone marrow (FIG. 9d), these data strongly suggest that CZCs are CTCs shed from primary tumors through a process regulated by NALCN. In the immediate 5-week period following tamoxifen recombination, similar levels of circulating CZCs were observed among P1^{KP} and V1^{KP} mice that were Nalcn^{+/+}, Nalcn^{+/Flx} or Nalcn^{Flx/Flx}, suggesting that Nalcn regulates cell shedding as a late event (FIG. 9e,f and Rahrmann et al 2022—Supplementary Table 13); however, the time taken for lineage tracing to reach steady state in our mice may underestimate CZC numbers at early time points.

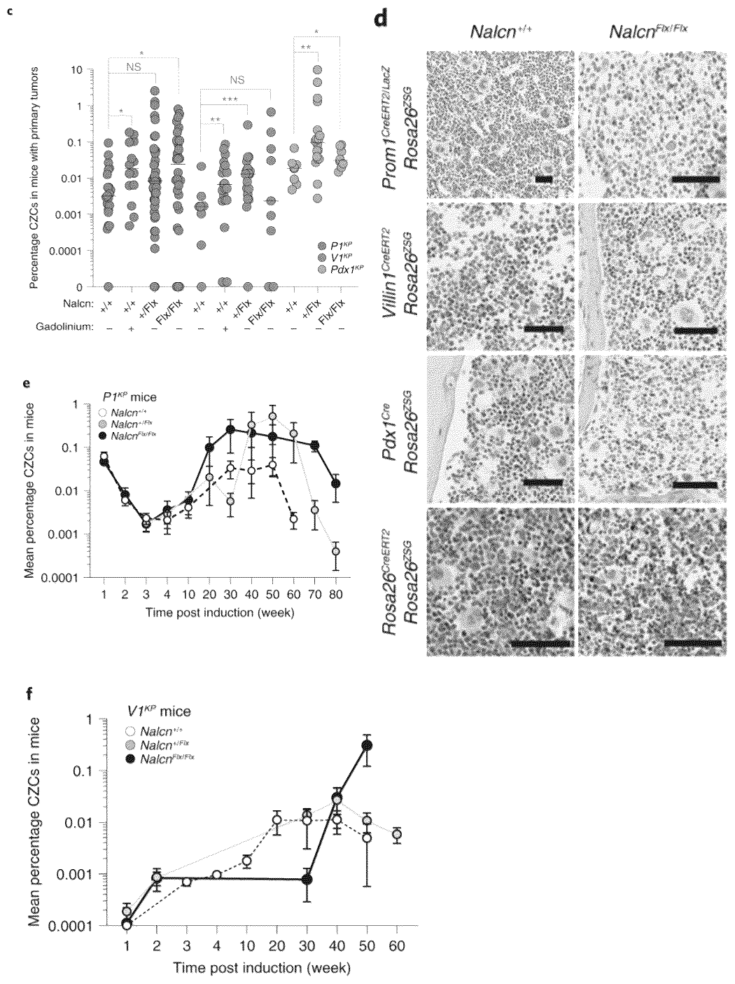

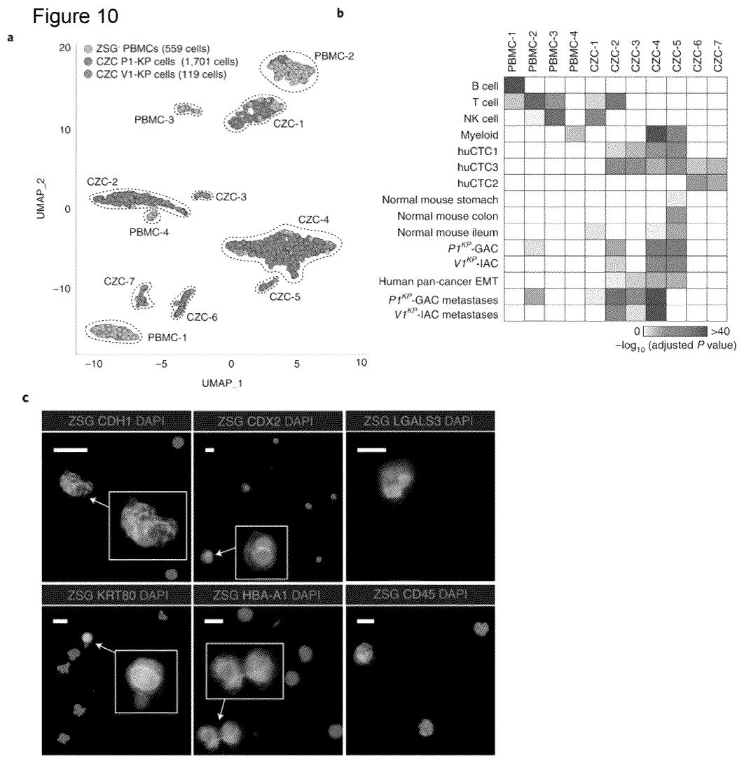

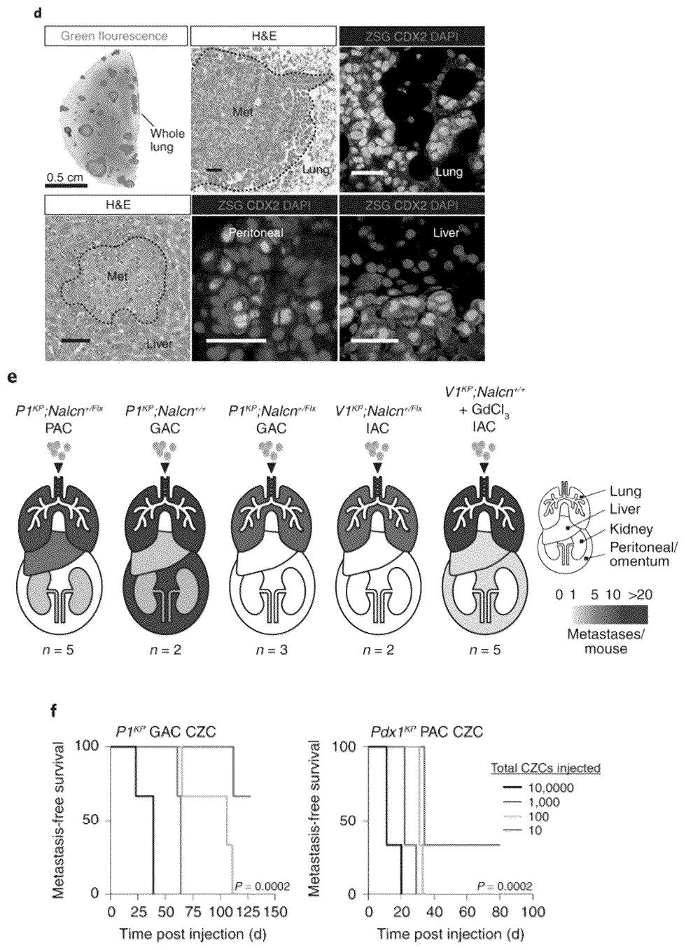

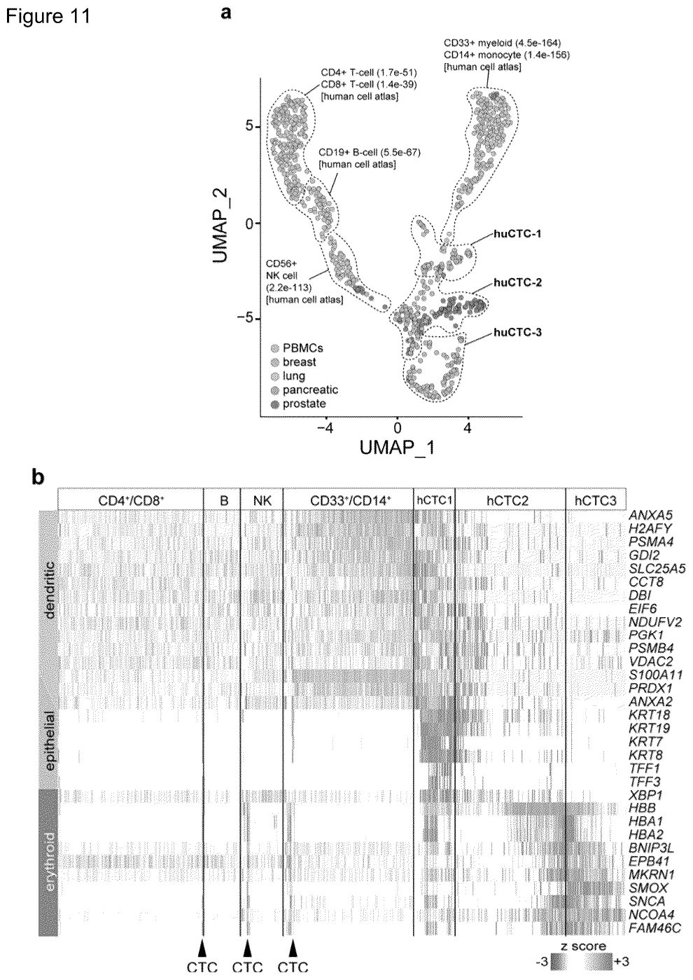

To better understand the origin of CZCs, we generated single-cell RNA sequence profiles of CZCs isolated from mice with P1^{KP}-GAC (n=1,701 cells) or V1KP-IAC (n=119), as well as peripheral blood mononuclear cells (PBMCs, n=559; FIG. 10a), and compared these with published single-cell RNA sequence profiles of human breast, lung, pancreatic and prostate CTCs (n=360) and PBMCs (n=500)31,32,33,34,35,36. Human CTCs comprised three overlapping clusters (FIG. 11 a-c and Rahrmann et al 2022—Supplementary Tables 14 and 15): ‘huCTC1’ (enriched with cancer metastasis, EMT and epithelial gene sets); huCTC3 (enriched with early-erythroid and EMT gene sets); and huCTC2 (sharing profiles of huCTC1 and huCTC3). huCTC1-3 expressed p-globin (HBB)—a survival factor for human CTCs33—as well as HBA1, HBA2 and HBD. Mouse CZCs formed seven clusters whose transcriptomes significantly matched huCTC1 (mCZC2-7), huCTC2 (mCZC2, 3, 5-7) and huCTC3 (mCZC2-7), and included orthologs of HBA1, HBA2 (Hba-a1, Hba-a2), HBB (Hbb-bs, Hbb-bt), ANXA2 and LGALS3, as well as genes expressed in normal and malignant stomach and small intestine (FIG. 10a,b, and FIG. 11c-g and Rahrmann et al 2022—Supplementary Tables 16 and 17). Normalization and Uniform Manifold Approximation and Projections (UMAP) of all single-cell RNA sequence profiles also revealed significant overlap in mouse CZC and human CTC transcriptomes, especially those enriched for CD71+ erythroid genes (FIG. 11e-g and Rahrmann et al 2022—Supplementary Table 18). Coimmunofluorescence of peripheral blood smears taken from mice with V1KP-IAC and P1^{KP}-GAC confirmed CZC expression of HBA-A1, LGALS3, and epithelial cell markers (KRT80, CDH1) and CDX2 that marks intestinal epithelium (FIG. 10c). PBMCs did not express these markers but did express markers of PBMCs (for example, CD45).

To test directly whether CZCs possess metastatic potential, we injected separate aliquots of 25,000 CZCs isolated from mice with P1^{KP}-PAC, P1^{KP}-GAC or V1^{KP}-IAC into the tail veins of immunocompromised mice. Within 75 d, all mice developed numerous ZSG⁺ metastases in the lungs, liver, kidneys and/or peritoneum (FIG. 10d,e and Rahrmann et al 2022—Supplementary Table 19). Similar studies with increasing cell dilutions showed that as few as ten CZCs were required to generate metastasis (FIG. 1 Of and Rahrmann et al 2022—Supplementary Table 19). Thus, CZCs are highly enriched for CTCs that recapitulate the transcriptome of human CTCs and are shed into the peripheral blood through a process regulated by Nalcn.

### Example 9—NALCN and Circulating Noncancer Cells

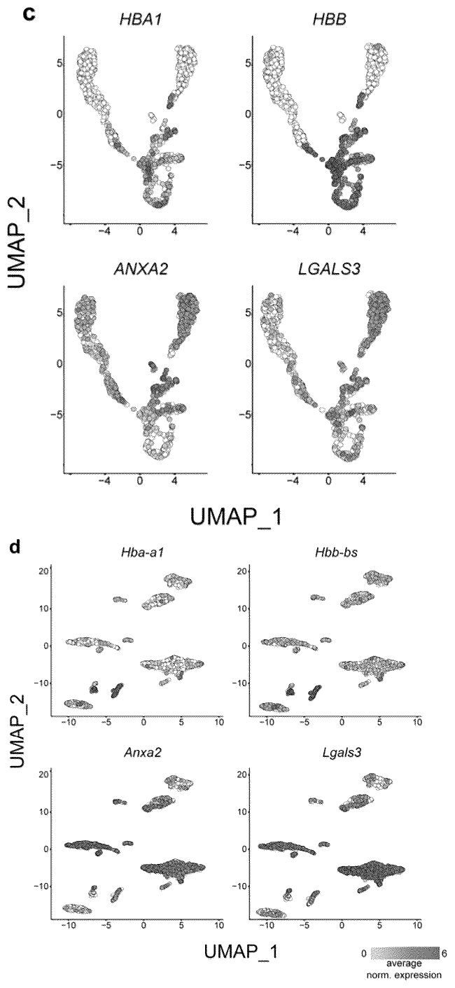

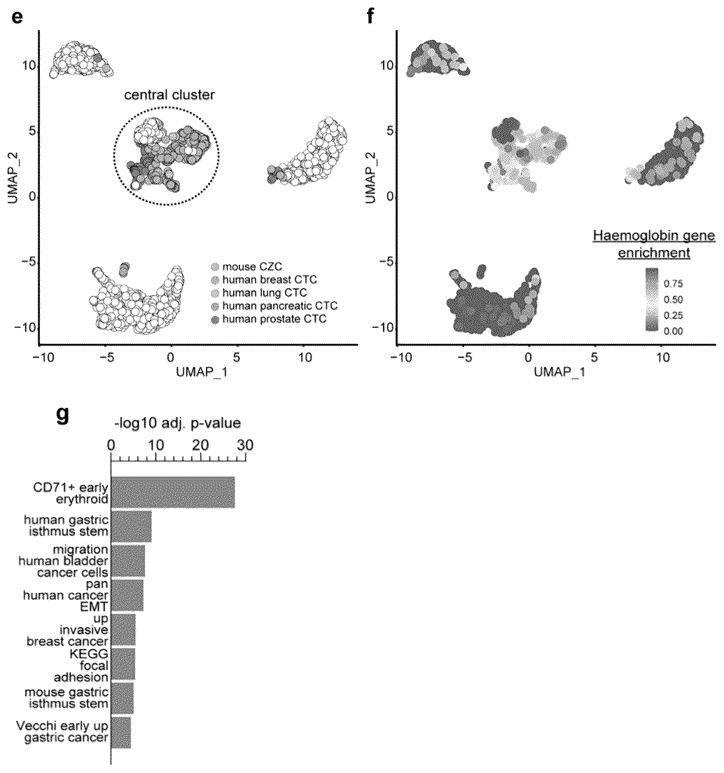

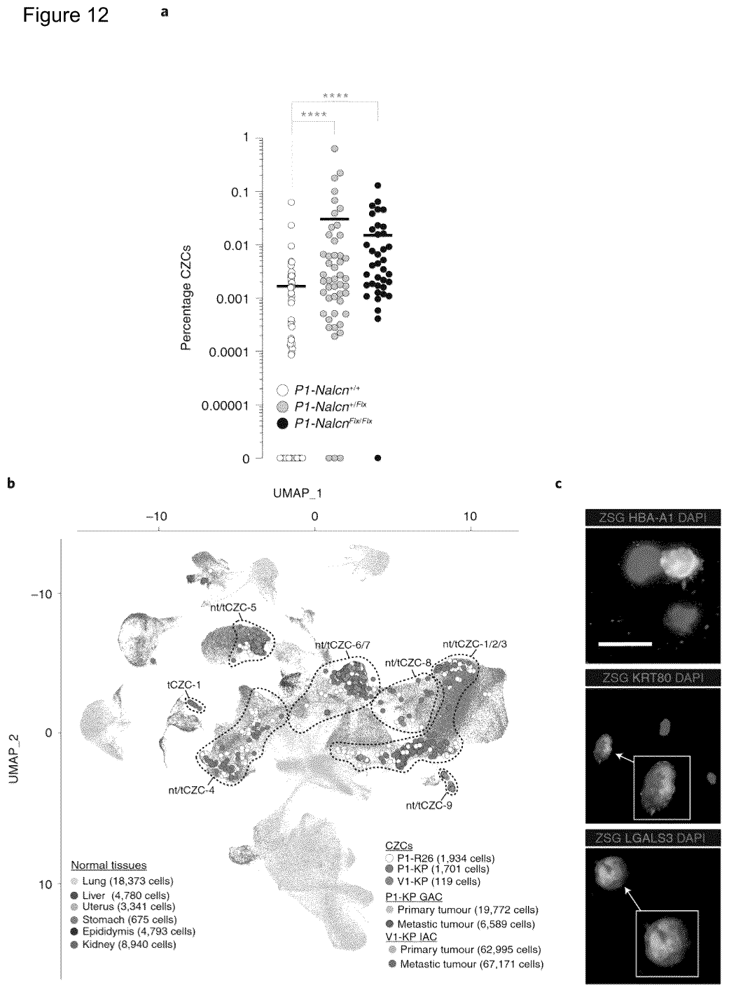

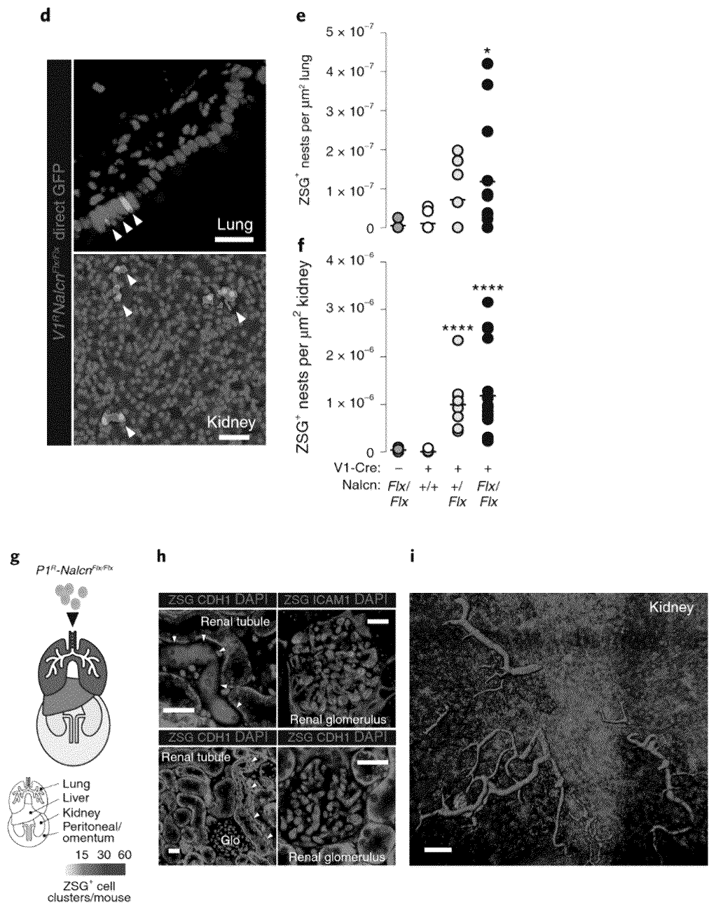

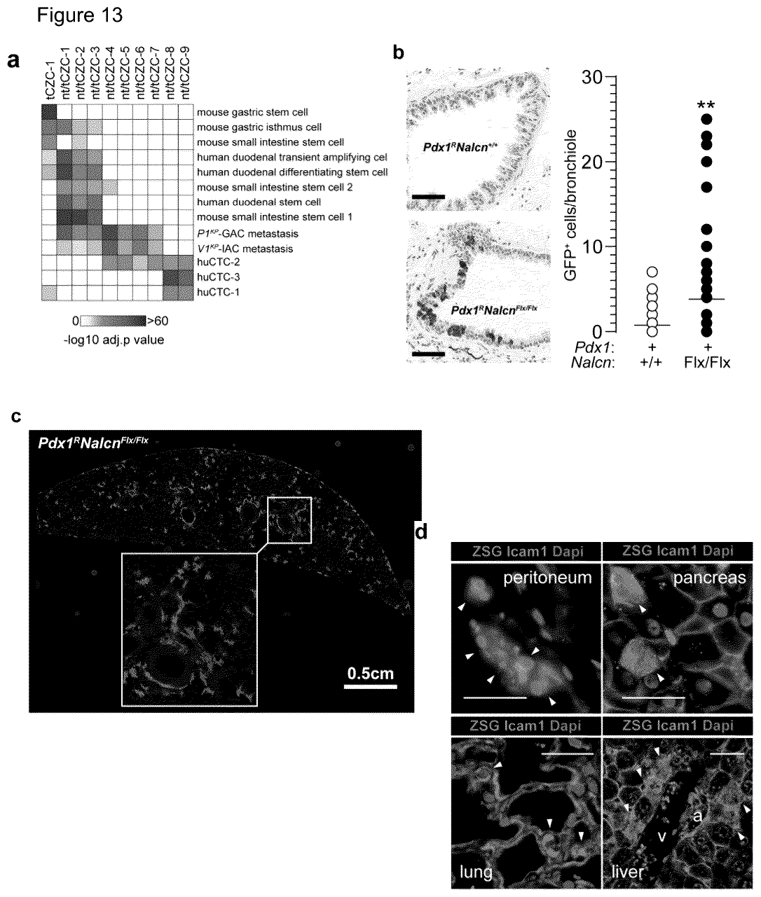

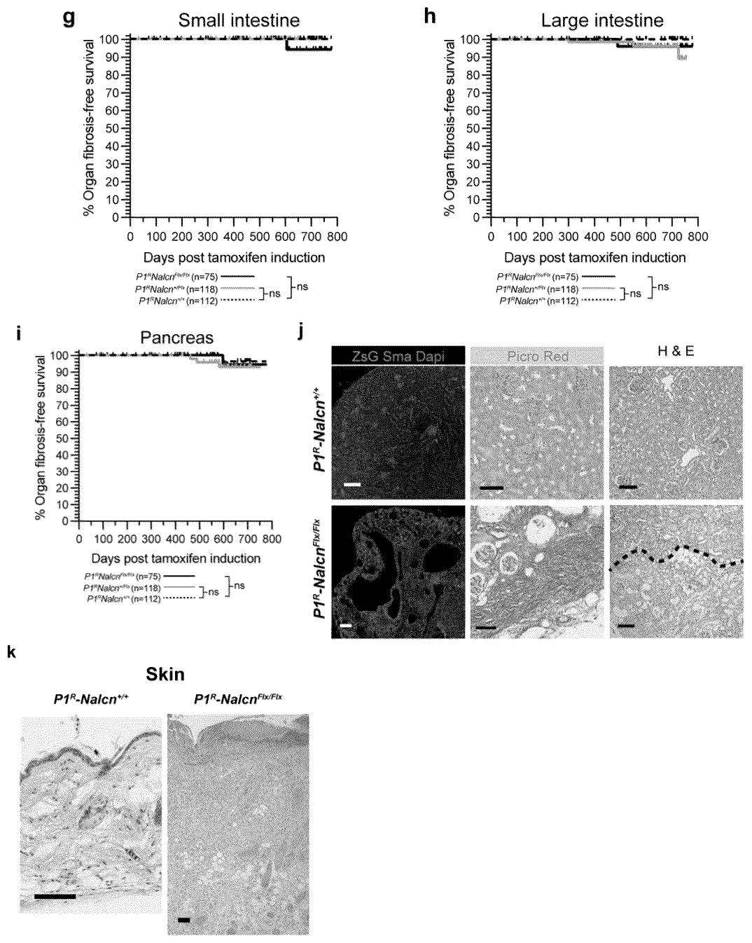

Preventing CTC shedding into the peripheral blood could stop metastasis, but disentangling this process from the complex cascade of tumorigenesis has proved challenging. Deletion of Nalcn from freshly isolated P1; Nalcn^{Flx/Flx} gastric stem cells that lacked oncogenic alleles, rapidly upregulated genes associated with invasion (for example, Mmp7, Mmp9, Mmp10 and Mmp19) and gastric EMT (for example, Zeb1, Fstl1, Sparc, Sfrp4, Cdh6 and Timp3; Rahrmann et al 2022—Supplementary Tables 20 and 21), suggesting NALCN might regulate cell shedding from solid tissues independent of transformation. To test this, we looked for CZCs in the peripheral blood of Prom1^{CreERT2/Laz}; Rosa26^{ZSG}; Nalcn^{+/+} (P1^{R}Nalcn^{+/+}, n=87), P1^{R}Nalcn^{+/Flx} (n=50) and P1^{R}Nalcn^{Flx/Flx} (n=37) mice that lacked oncogenic alleles and never developed tumors (Rahrmann et al 2022—Supplementary Table 13). Remarkably, deletion of Nalcn increased the numbers of CZCs in these mice to levels similar to those observed in tumor-bearing animals (FIGS. 12b,c and 15a). Single-cell RNA sequencing (SCS) profiles of CZCs isolated from nontumor-bearing (ntCZC) mice co-clustered with CZCs from tumor-bearing animals (tCZC; FIG. 13b). The great majority of tCZC and ntCZC SCSs did not cluster with SCS profiles of primary IACs, GACs or normal tissues, but with SCS profiles of metastases (FIG. 13b and Rahrmann et al 2022—Supplementary Table 22). SCS profiles of both tCZCs and ntCZCs matched those of human CTCs and, similar to human CTCs2, expressed genes associated with stem and progenitor cells; although tCZCs were relatively more enriched for metastasis and invasion-associated gene sets (FIG. 13a and Rahrmann et al 2022—Supplementary Tables 23 and 24). Coimmunofluorescence of blood smears confirmed that both ntCZCs and tCZCs share markers of huCTCs, including HBA-A1 (FIGS. 13c and 15c).

To understand the fate of ntCZCs, we looked for ZSG⁺ cells in the lungs and kidneys of aged V1^{R} and Pdx1^{R} Nalcn^{+/+}, Nalcn^{+/Flx} and/or Nalcn^{Flx/Flx} mice. Remarkably, ZSG⁺ cell clusters were readily detected in these organs in Nalcn-deleted animals, but were absent or detected at significantly lower levels in Nalcn^{+/+} mice, suggesting that ntCZCs traffic to, and embed within, distant organs (FIG. 12d-f and FIG. 13b,c). To test this more directly, we injected separate aliquots of 25,000 ntCZCs isolated from P1^{R}Nalcn^{Flx/Flx} mice into the tail veins of six immunocompromised mice. All recipient mice remained clinically well after an average of 100 d, but contained numerous ZSG⁺/Cdh1⁺/Icam1⁺ donor-cell clusters within their lungs, liver, kidneys and peritoneum at a frequency similar to metastatic tumors formed by tail-vein injections of tCZCs (FIGS. 10e, 12g-i and FIG. 10d). Trafficked ntCZCs formed apparently normal structures in target organs, the most extreme example being kidney glomeruli and tubules (FIG. 12h,i). Thus, NALCN regulates cell shedding from solid tissues independent of cancer, divorcing this process from tumorigenesis and unmasking an oncogene-independent metastatic pathway.

### Example 10—NALCN-Blockade Causes Systemic Fibrosis

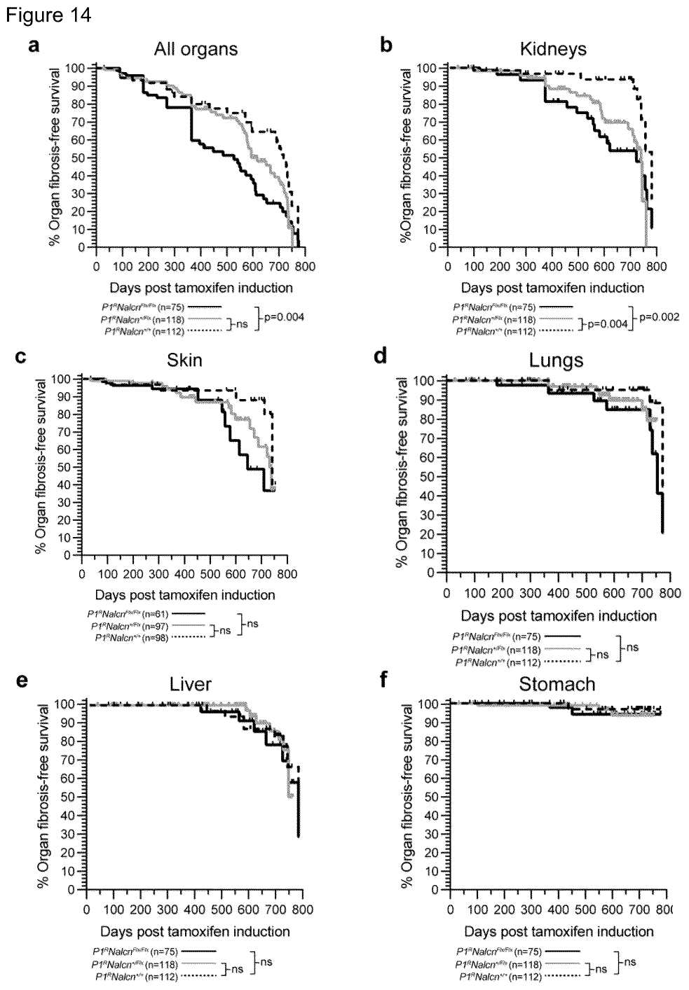

Although P1^{R}Nalcn^{+/Flx} (n=118) and P1^{R}Nalcn^{Flx/Flx} (n=112) mice did not develop cancer, whole-body autopsy of these mice revealed severe kidney and skin fibrosis relative to P1^{R}Nalcn^{+/+} (n=65) mice (Rahrmann et al 2022—Supplementary Table 25 and FIG. 14). This pathology arose after 2400 d and replicated that of gadolinium-induced systemic fibrosis (GISF), a debilitating condition manifested by severe organ fibrosis following administration of gadolinium-based contrast agents37. How gadolinium-based contrast agents cause GISF is unknown, but suggested mechanisms include tissue retention of gadolinium-based contrast agents and the mobilization and recruitment of bone marrow-derived fibrocytes38. Our data suggest strongly that blockade of the NALCN channel by gadolinium mobilizes epithelial cells in a variety of epithelial tissues that traffic to the kidney and other organs, eventually eliciting a fibrotic response, causing GISF.

Developing antimetastatic therapies has proven difficult because targets in primary tumors that drive metastasis have proved hard to find2. By divorcing the process of CTC shedding from ‘upstream’ tumorigenesis, our data unmask manipulation of NALCN function as a promising new approach to block metastasis. In particular, drugs capable of reopening the NALCN channel might be effective antimetastatic therapies. Precedent for this approach is provided by drugs that open the chloride channel mutated in cystic fibrosis39. If successful, such agents may also be useful for treating GISF.

It is important to note that our observations are based on deleting Nalcn from mouse tissues, whereas NALCN in human cancers is affected predominantly by nonsynonymous mutations. Although our in silico modeling suggests strongly that these cancer-associated mutations close the NALCN channel, it will be important to demonstrate this functionally by modeling nonsynonymous Nalcn mutations in vivo. These studies should also include testing in patient-derived xenografts of gastric, colon and other cancers to confirm that NALCN regulates trafficking of human as well as mouse cells.

Loss-of-function mutations in NALCN may also help explain various enigmatic features of human cancer. Metastases can emerge many years after removal of a localized cancer40, or in the absence of a primary tumor4l. Loss of NALCN function in our mice caused an abundant and persistent shedding of cells that embed in distant organs, even in the absence of a primary tumor. Because human epithelial tissues contain fields of phenotypically normal cells that harbor oncogenic mutations42,43, then loss of NALCN function in these cells could provide a source of CTCs that form metastases in the absence of a primary tumor, or long after a primary tumor has been removed. It is likely that such cells would need to acquire additional mutations to form tumors at the metastatic site, compatible with the relative rarity of these phenomena. Our data may also explain why CTCs have been found in the bone marrow of patients who lack metastases. Although these cells could represent ‘dormant’ CTCs, as previously suggested3, equivalent to ntCZCs in our mice, they may be shed from nontransformed epithelia that have lost NALCN function, but not gained the ability to form metastatic tumors. Our serial analysis of CZCs in mice suggest that cell shedding following NALCN loss-of-function is a late, rather than early, event; although NALCN mutations could promote both linear and parallel progression models of cancer44.

Our data also provide clues as to how NALCN might regulate epithelial cell shedding. We observed upregulation of genes associated with EMT and invasion within 72 h of deleting Nalcn from normal gastric stem cells; suggesting that this channel might regulate gene transcription in a similar manner to that reported for calcium ion channels6,45. Our electrophysiology studies demonstrate that GAC cells possess a NALCN-mediated current. However, more detailed electrophysiology studies are required to determine the precise mechanism by which NALCN regulates gene expression and cell shedding and whether this involves maintenance of the resting membrane potential.

The development of renal and skin fibrosis reminiscent of GISF in aged Nalcn-deleted mice, pinpoint NALCN channel blockade as the likely cause of this debilitating condition. P1^{KP} mice succumbed to cancer well before the onset of organ fibrosis in P1R mice, and Nalcn deletion in P1R mice did not induce stomach, intestine, lung, pancreas or liver fibrosis-principal sites of primary and metastatic tumors in P1^{KP} mice. Thus, fibrosis is unlikely to have contributed to metastasis in Nalcn-deleted mice. However, because limited exposure to gadolinium can induce GISF in humans, it is a note of concern that gadolinium-contrast imaging of cancer patients could accelerate metastasis.

### Methods

### Culture of Stomach Stem Cells

Gastric glands were isolated46 by perfusing mice with 30 mM EDTA/PBS, stomach removal and scraping pyloric mucosa into 10 mM EDTA/PBS at 4° C. Dissociated, filtered and resuspended cells were placed in Matrigel (catalog number 354230, BD Biosciences) and culture medium: advanced DMEM/F12 (catalog number 31330038, Thermo Fisher Scientific), B27 (catalog number 12587010, Thermo Fisher Scientific), N2 (catalog number A1370701, Thermo Fisher Scientific), N-acetylcysteine (catalog number A9165, Sigma-Aldrich) and 10 nM gastrin (catalog number G9145, Sigma-Aldrich) containing growth factors (50 ng ml⁻¹ EGF (PeproTech), 1 mg ml⁻¹ R-spondin1 (catalog number 120-38, PeproTech), 100 ng ml⁻¹ Noggin (catalog number 250-38, PeproTech), 100 ng ml⁻¹ FGF10 (catalog number 100-26, PeproTech) and Wnt3A conditioned media (L Wnt-3A, catalog number ATCC-CRL-2647, American Type Culture Collection). Gastric spheres were passaged by dispase (catalog number D4818, Sigma-Aldrich) digestion and dissociation into single cells (StemPro Accutase, Life Technologies). Gadolinium (catalog number 439770, Sigma-Aldrich) was diluted in the culture medium and overlaid on Matrigel embedded cells (Rahrmann et al 2022—Supplementary Tables 26 and 27).

### Lentiviral Production and Transduction

Nalcn-shRNA lentivirus was produced as described previously47. Three shRNAs per target (two open reading frames one 3′-untranslated region) were cloned into pFUGWH1-RFPTurbo and cotransfected with plasmids pVSV-G and pCMVd8.9 into 293FT (Thermo Fisher Scientific, catalog number R70007) cells. NALCN cDNA (NM_052867) was from OriGene (catalog number RC217074). In total 2×10⁴ gastric cells were mixed with lentiviruses (20 particles per cell) plated in Matrigel. Transduced red fluorescence⁺ (shRNA) or green fluorescence⁺ (cDNA) cells were sorted using a Becton Dickinson Aria II Cell Sorter (Rahrmann et al 2022—Supplementary Tables 26 and 28).

### Whole-Cell Electrophysiology

The NALCN channel current was measured as reported48. Whole-cell recordings were obtained from stomach tumor cells on 12-mm cover slips coated with Matrigel at a density of 25,000 cells per ml and superfused (2-3 ml min⁻¹) with warm (30-32° C.) recording solution containing 120 mM NaCl, 5 mM CsCl, 2.5 mM KCl, 2 mM CaCl₂), 2 mM MgCl₂, 1.25 mM NaH₂PO₄, 26 mM NaHCO₃, 20 mM glucose and 1 M tetrodotoxin (300-310 mOsm), with 95% O₂/5% CO₂. Patch pipettes (open pipette resistance, 3-4 MO) were filled with an internal solution containing 125 mM CsMeSO₃, 2 mM CsCl, 10 mM HEPES, 0.1 mM EGTA, 4 mM MgATP, 0.3 mM NaGTP, 10 mM Na₂ creatine phosphate, 5 mM QX-314 and 5 mM tetraethylammonium Cl (pH 7.4, adjusted with CsOH, 290-295 mOsm). Tetrodotoxin and QX-314 were included to block voltage-sensitive sodium channels in recorded cells, whereas cesium and tetraethylammonium Cl blocked voltage-sensitive potassium channels. Voltage-clamp recordings were made using a Multiclamp 700B (Molecular Devices), digitized (10 kHz; DigiData 1322A, Molecular Devices) and recorded using pCLAMP v.10.0 software (Molecular Devices). In all experiments, membrane potentials were corrected for a liquid junction potential of −10 mV. After forming a gigaseal onto a cell and rupturing the cell membrane, tumor cell membrane potential was held at −70 mV. Cell membrane capacitance, membrane resistance and pipette access resistance were then measured with the pCLAMP cell membrane test function. Recordings were excluded if pipette access resistance was higher than 20 MO or if access resistance changed by more than 20% during the experiment. After cell membrane resistance had stabilized, membrane potential was then stepped to 0 mV for 100 ms followed by a series of 250 ms voltage steps from −80 mV to +80 mV in 20-mV increments and the current response to these voltage steps was recorded. GdCl₃ (100 μM) was then applied to the bath solution to eliminate the voltage-independent ‘leak’ current associated with Nalcn. Calculation of the Nalcn current was performed offline by subtracting the current response in GdCl₃ from the previous GdCl3-free current recording. Tumor cell Nalcn current density was determined by dividing the Nalcn current by cell membrane capacitance. To verify successful expression of the RFP⁺ (Nalcn^{shRNA}) or GFP⁺ (NALCN^{cDNA}) construct, cells were imaged with two-photon laser scanning microscopy (Prairie Technologies) using a Ti:sapphire Chameleon Ultra femtosecond-pulsed laser (Coherent), and ×60 (0.9 NA) water-immersion infrared objective (Olympus). Red fluorescent protein was visualized using an excitation wavelength of 1030 nM, whereas green fluorescent protein (GFP) was visualized using an excitation wavelength of 820 nM (Rahrmann et al 2022—Supplementary Tables 26 and 28).

### Gastric Adenocarcinoma Allografts

P1^{KP}-GAC orthotopic and flank allografts were generated under protocols approved by the Institutional Animal Care and Use Committee of St. Jude Children's Research Hospital (IACUC-SJ). For orthotopic grafts, a longitudinal abdominal incision was made to expose the pyloric valve of CD-Foxn1^{NU} mice and 2×10⁵ freshly dissociated P1^{KP}-GAC cells suspended in Matrigel and fast green (Santa Cruz) were injected into the pyloric stomach epithelium. The wound was closed and mice were monitored daily for tumor development. Under veterinary guidance and IACUC-SJ approved measures, animals reaching humane end points were immediately euthanized and a full autopsy completed (Rahrmann et al 2022—Supplementary Tables 26 and 29).

Generation of Nalcn^{Flx} allele

Mice were derived from targeted embryonic stem cells (ESCs) (UCDAVIS KOMP Repository Knockout Mouse Project clone EPD0383_5_C01). ESCs were screened using KOMP PCR strategies for Nalcntm1a(KOMP)Wstsi. ESCs were implanted into recipient C57/Bl6 mice in accordance with protocols approved by IACUC-SJ. Wild-type Nalcn and NalcnFlx alleles were detected using standard PCR and primers (UCDAVIS KOMP Repository Knockout Mouse Project clone EPD0383_5_C01). Nalcn RNA expression was quantified by quantitative PCR (qPCR) with reverse transcription and a Bio-Rad CFX96 Touch Real-Time PCR Detection System with primers (see Rahrmann et al 2022—Supplementary Tables 26 and 29-31 for details on animals and oligonucleotide sequences).

### Tumorigenesis and Surveillance

All animal studies within the United Kingdom (UK) were performed under the Animals (Scientific Procedures) Act 1986 in accordance with UK Home Office licenses (Project License 70-8823, P47AE7E47, PP7834816) and approved by the Cancer Research UK (CRUK) Cambridge Institute Animal Welfare and Ethical Review Board. Mice were housed in individually ventilated cages with wood chip bedding and nestlets with environmental enrichment (cardboard fun tunnels and chew blocks) under a 12 h light/dark cycle at 21±2° C. and 55%±10% humidity. Diet was irradiated LabDiet 5R58 with ad libitum water. Animals carrying the modified Nalcn allele were bred to RosaFLPe-expressing mice to remove LacZ and Neo cassette. Animals with complete recombination were bred with: Prom1C-L29; Nestin-cre49; Rosa-CreERT50; villin-CreER25; Pdx1-cre28; RosaZSG51; and KrasG12D/+52, Trp53f/x53. Cre-recombination was activated by dosing with 1 mg of tamoxifen per 40 g (body weight) at P3 or 8 mg tamoxifen per 40 g (body weight) at P60. Mice were maintained for up to 2 years and full-body autopsy was performed as described4 at humane end points or the indicated time point, whichever was first. All tissues were inspected for macroscopic tumors with direct green fluorescence detection. Tissues were formalin fixed, paraffin embedded with portions also snap frozen or used for tissue dissociation for sequencing (Rahrmann et al 2022—Supplementary Tables 26 and 29).

### Histology

Hematoxylin and eosin (H&E) staining was performed using standard procedures (catalog number 7221, 7111, Thermo Fisher Scientific). Fibrosis was assessed using modified Masson's trichrome and Picrosirius Red stains. Immunohistochemistry was performed using standard procedures and primary antibodies: Ki67 (catalog number IHC-00375, Bethyl Laboratories, 1:1,000), ZSG (catalog number 632474, Clontech, 1:2,000), pan cytokeratin (AE1/AE3) (catalog number 901-011-091620, BioCare Medical, 1:100), CK5 (catalog number ab52635, Abcam, 1:100), vimentin (catalog number 5741 S, Cell Signaling Technology, 1:200), cleaved caspase 3 (catalog number 9664, Cell Signaling Technology, 1:200), CD31 (catalog number 77699, Cell Signaling Technology, 1:100), a-smooth muscle actin (catalog number ab5694, Abcam 1:500), CD45 (catalog number ab25386, Abcam, 5 μg ml⁻¹). Secondary antibodies were antirabbit poly-horseradish peroxidase-IgG (included in kit) or rabbit antirat (catalog number A110-322A, Bethyl Laboratories, 1:250). Digital images of entire tissue sections were captured using the Leica Aperio AT2 digital scanner (×40, resolution 0.25 μM per pixel), viewed using the Leica Aperio Image Scope v.12.3.2.8013 and quantified by HALO (Indica Labs) image analysis (Rahrmann et al 2022—Supplementary Tables 28 and 33).

For immunofluorescence, tissue sections were incubated with primary antibodies: rhodamine-labeled DBA (catalog number RL-1032, Vector Laboratories, 1:100), rhodamine-labeled UEA I (catalog number RL-1062, Vector Laboratories, 1:100), ZSG (catalog number TA180002, Origene, 1:1,000), CK7 (catalog number ab181598, Abcam, 1:200), CK20 (catalog number ab97511, Abcam, 1:200), E-cadherin (catalog number AF748, R&D Systems, 1:100), N-cadherin (catalog number 13116, Cell Signaling Technology, 1:100), Icam1 (catalog number ab179707, Abcam, 1:100), Cdx2 (catalog number ab76541, Abcam, 1:100), Krt80 (catalog number 16835-1-AP, ProteinTech, 1:100), Hba-a1 (catalog number ab92492, Abcam, 1:100), Lgals3 (catalog number ab209344, Abcam, 1:200), CD45 (catalog number ab10558, Abcam, 1:200). Secondary antibodies included Alexa 488, 594 and 647 (catalog numbers A-11055, A-21207 and A-31571, Thermo Fisher Scientific, 1:500). Sections were counterstained (4,6-diamidino-2-phenylindole (DAPI); catalog number 4083, Cell Signaling, 1:10,000) and images captured using a Zeiss ImagerM2 and Apotome microscope or Zeiss Axioscan.Z1 (Zeiss) at x40 magnification and processed using ZEN2.3 (Zeiss) software (Rahrmann et al 2022—Supplementary Tables 28 and 33). Nalcn RNA expression was detected in formalin-fixed, paraffin-embedded sections using the Advanced Cell Diagnostics (ACD) RNAscope 2.5 LS Reagent Kit-RED (ACD, catalog number 322150) and RNAscope 2.5 LS Mm Nalcn (ACD, catalog number 415168). Probe hybridization and signal amplification were performed according to the manufacturer's instructions. Fast Red detection of mouse Nalcn was performed was performed on the Bond Rx using the Bond Polymer Refine Red Detection Kit (Leica Biosystems, catalog number DS9390) according to the manufacturer's protocol. Whole-tissue sections were imaged on the Aperio AT2 (Leica Biosystems) and analyzed as for immunohistochemistry using HALO (Indica Labs) imaging analysis software. p-Galactosidase staining was performed exactly as described4 (Rahrmann et al 2022—Supplementary Tables 26, 28 and 30).

Histological review, primary and metastatic tumor classification were performed by performed by expert pathologists (P. Vogel and B. Mahler-Araujo) blinded to mouse genotype and clinical history. The numbers of ZSG⁺ cell clusters or metastases were counted in each organ in each mouse. Tissue fibrosis was assessed by expert pathologist R. Nazarian using sections stained with H&E, Masson's trichrome and Picrosirius Red.

### Whole-Tissue Imaging

Kidneys were exsanguinated, perfused with PBS and 4% PFA by PBS washes and immersion reagent 1 a (150 g of ultrapure water, 20 g of Triton X-100 (catalog number 10254583, Thermo Fisher Scientific), 10 g of 100% solution of N,N,N′,N′-tetrakis (2-hydroxypropyl)ethylenediamine (catalog number 122262, Sigma), 20 g of urea (catalog number 140750010, ACROS Organics), 1 ml of 5 M NaCl) containing 10 μM DAPI (catalog number 4083; Cell Signaling Technology) at 37° C. and 80 r.p.m. The solution was exchanged every 2 d until the tissue was cleared. Cleared tissues were washed and immersed in 50% PBS/50% reagent 2 (15 g of ultrapure water, 50 g of sucrose (catalog number 220900010, ACROS Organics), 25 g of urea (catalog number 140750010, ACROS Organics), 10 g of 2,2,2-nitrilotriethanol (catalog number 90279, Sigma)) for 6 h (room temperature, with gentle shaking) followed by immersion in 100% reagent 2 (10 ml) for 1 d (room temperature). Tissues were mounted and scanned on a TCS SP5 confocal laser scanning microscope (Leica) at ×10 objective for DAPI and endogenous expression of ZSG. Images were processed using Imaris x64 v.9.3.0 software (Oxford Instruments) (Rahrmann et al 2022—Supplementary Tables 26, 28 and 30).

Serial two-photon tomography imaging was performed on a TissueCyte 1000 instrument (TissueVision) in which a series of mosaic two-dimensional images are taken of the tissue, followed by physical sectioning with a vibratome and a subsequent round of imaging. This continues in an automated fashion, generating 15 μm serial two-photon tomography sections that can be mounted on standard microscopy slides, imaged by Axioscan fluorescence scanning (Zeiss) for section identification and realignment. Fiducial agarose marker beads labeled with GFP are distributed throughout the embedding medium to help in the realignment of the samples for consequent use (Rahrmann et al 2022—Supplementary Tables 26, 28 and 30).

### Harvesting and Injection of Circulating ZSG Cells

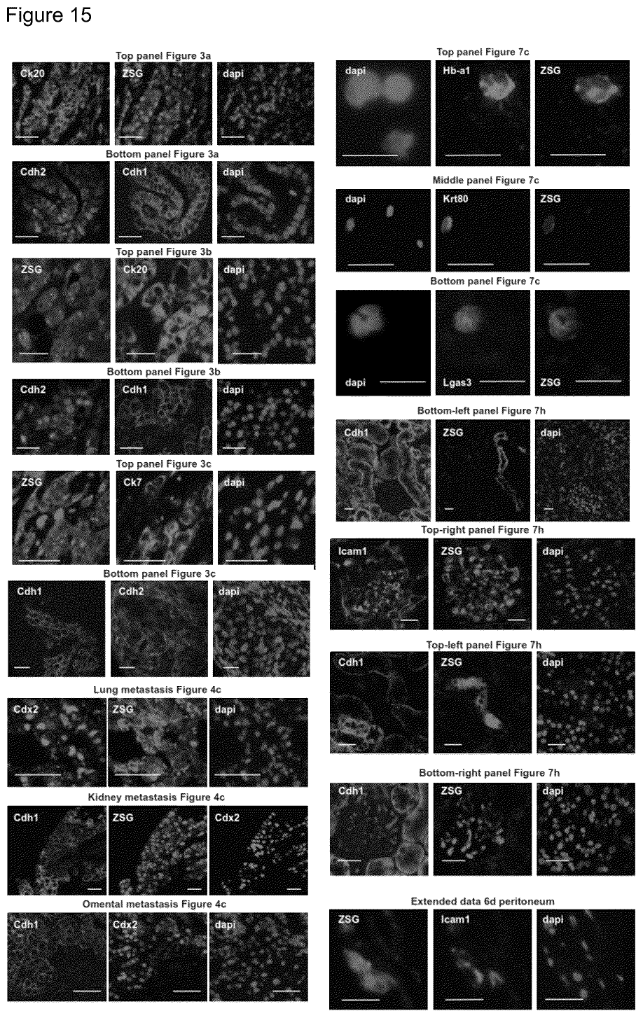

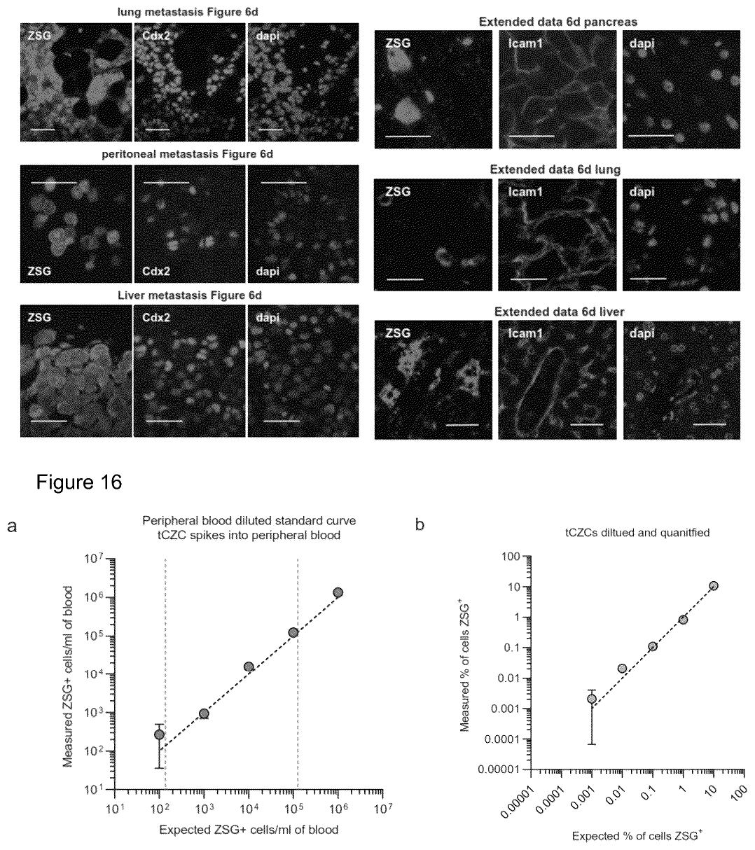

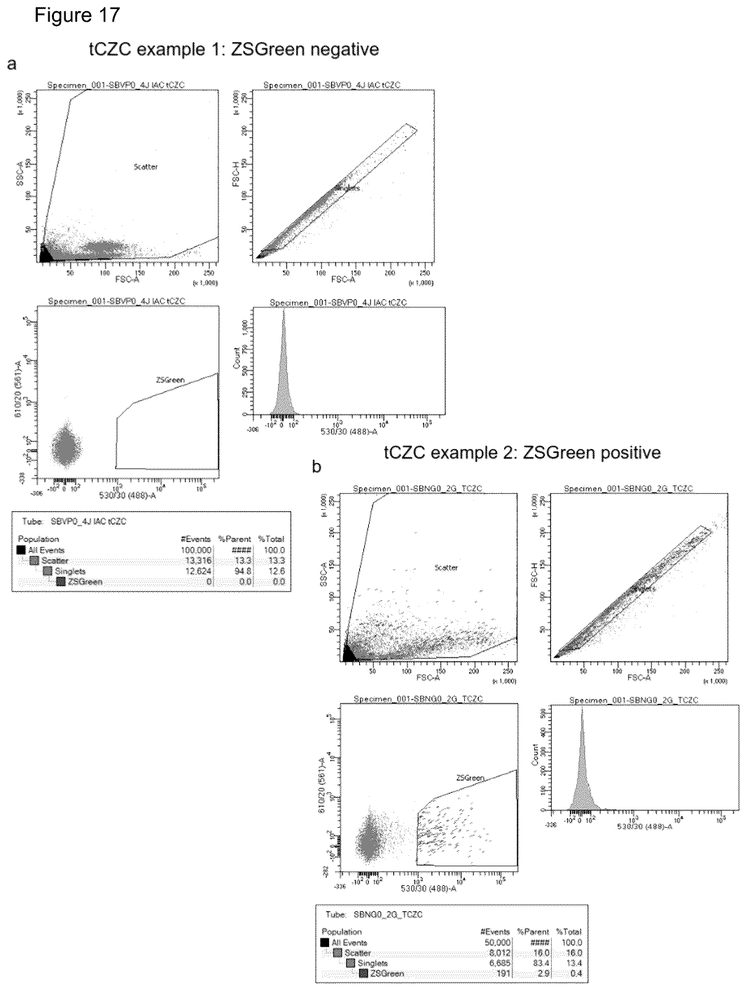

Peripheral blood (500 μl to 1 ml) was harvested from mice at autopsy into 10 μl of 0.5 M EDTA, diluted in PBS and assessed by MACSQuant Analyzer (Miltenyi Biotech Inc.) for ZSG expression (525/50 nm (FITC) versus 614/50 nm (propidium iodide)). Cells for SCS and tail-vein injection were sorted using a BD FACSAria II Cell Sorter (BD Biosciences) excitation at 525/50 nm (FITC) versus 614/50 nm (propidium iodide). Nontamoxifen-induced mouse peripheral blood served as a negative control to set gate parameters (FIGS. 16 and 17). Some 25,000 ZSG⁺ cells were sorted and injected into recipient NOD SCID gamma mice (Charles River) and aged. For serial dilution assessment of tCZC metastasis initiation, tCZCs were isolated from donor tumor-bearing animals via FACS based on ZSG expression and placed into culture medium. Culture medium was as follows: Advanced DMEM/F12 (catalog number 31330038, Thermo Fisher Scientific), 2 mM L-glutamine (catalog number 25030024, Thermo Fisher Scientific), B27 (catalog number 12587010, Thermo Fisher Scientific) and N2 (catalog number A1370701, Thermo Fisher Scientific), containing growth factors (50 ng ml⁻¹ epidermal growth factor (PeproTech), 100 ng ml⁻¹ basic fibroblast growth factor (catalog number 100-18c, PeproTech) and 1% FBS (catalog number 10500064, Thermo Fisher Scientific). Cells were grown at 37° C. in 5% CO₂.

Recipient NOD SCID gamma mice (Charles River) were injected with either 10, 100, 1,000 or 10,000 tCZCs via tail-vein injection and aged. Full autopsy and tissue harvesting were performed as described above. Full autopsy and tissue harvesting were performed as described above (Rahrmann et al 2022—Supplementary Tables 26, 28 and 29).

### Bulk RNA Sequencing

Total RNA was extracted from tissues using Maxwell RSC miRNA Tissue Kit (catalog number AS1460, Promega). RNA quality was assessed using TapeStation System (catalog number 5067-5579, Agilent). RNA libraries and downstream sequencing were carried out as previously described54. The Illumina TruSeq stranded messenger RNA kit (catalog number 20020595, Illumina) was used to prepare RNA libraries and RNA quality confirmed using TapeStation (Agilent) and quantified using a KAPA qPCR library quantification kit for Illumina platforms (catalog number KK4873, KAPA Biosystems). Samples were normalized using the Agilent Bravo, pooled and sequenced on Illumina NovaSeq SP flowcell to generate single-end 50 bp reads at 20 million reads per sample.

Single-end 50 bp RNA reads were aligned to GRCm38 with HISAT2 (with default parameters). Each sample was sequenced across several lanes; per-lane BAM files were merged into per-sample BAM files. Quality control metrics were collected for each file, including duplication statistics and number of reads assigned to genes. Reads were counted on annotated features with subreads featureCounts, providing ‘total’, ‘aligned to the genome’ and ‘assigned to a gene’ (that is, included in the analysis) counts. Percentages of aligned bases were computed for several categories: coding, untranslated region, intronic and intergenic. Other quality control metrics were the percentage of reads on the correct strand, median coefficient of variation of coverage, median 5′ bias, median 3′ bias and the ratio of 5′ to 3′ coverage. Quality control also included an expression heatmap drawn using log 2-transformed counts. The log 2-transformed counts were generated from normalized counts using the log 2 function in R and counts function from DEseq2. Genes were regarded as displaying differential expression between sample cohorts if they displayed of ≥1 or ≤−1 log(fold difference) in expression levels with an adjusted P≤0.05 (Rahrmann et al 2022—Supplementary Tables 26, 28 and 30).

### Single-Cell RNA Sequencing

Animals were perfused with PBS followed by 100 U ml⁻¹ of collagenase type IV in HBSS with Ca²⁺ and Mg²⁺ (Life Technologies) media containing 3 mM CaCl₂. Whole organs were dissected, dissociated and placed into 2 ml of the appropriate dissociation buffer: lung and stomach were dissociated with 200 U ml⁻¹ of collagenase type IV (Sigma) and 100 μg μl⁻¹ of DNAse I (Roche) in HBSS with Ca²⁺ and Mg²⁺ (Life Technologies) media containing 3 mM CaCl₂); liver was dissociated with collagenase type 1 (100 U ml⁻¹), dispase (2.4 U ml⁻¹) DNAse 1 (100 μg ml⁻¹) in HBSS with Ca²⁺ and Mg²⁺ (Life Technologies) media containing 3 mM CaCl₂; kidney was dissociated with papain (20 U ml⁻¹) and DNAse I (100 mg ml⁻¹) in DMEM high glucose, 2 mM L-glutamine (Life Technologies) with 1× Pen-Strep and 10% FBS; uterus and epididymis were dissociated with collagenase type I (100 U ml⁻¹) and DNAse I (100 mg ml⁻¹) in in HBSS with Ca²⁺ and Mg²⁺ (Life Technologies) media containing 3 mM CaCl₂). Cells suspensions were filtered washed with HBSS without calcium and magnesium and centrifuged for 5 min at 300 g at 4° C. for 5 min.

Single-cell suspensions of solid tissues were multiplexed and labeled with Cell Hashing conjugates: antimouse hashtags from 0301 to 0315 (BioLegend) before sequencing. All nucleated cells and ZSG⁺ cells isolated from peripheral blood were not multiplexed but placed into a 10× Genomics pipeline. SCS libraries were prepared using Chromium Single Cell 3′ Library & Gel Bead Kit v.3, Chromium Chip B Kit and Chromium Single Cell 3′ Reagent Kits v.3 User Guide (manual CG000183 Rev A; 10× Genomics). Cell suspensions were loaded on the Chromium instrument with the expectation of collecting gel-bead emulsions containing single cells. RNA from the barcoded cells for each sample was subsequently reverse-transcribed in a C1000 Touch thermal cycler (Bio-Rad) and all subsequent steps to generate single-cell libraries were performed according to the manufacturer's protocol with no modifications (for most of the samples 12 cycles was used for cDNA amplification, 16 for samples with very low cell concentration). cDNA quality and quantity were measured with Agilent TapeStation 4200 (High Sensitivity D5000 ScreenTape) after which 25% of material was used for preparation of the gene expression library. Library quality was confirmed with Agilent TapeStation 4200 (High Sensitivity D1000 ScreenTape to evaluate library sizes) and Qubit 4.0 Fluorometer (Qubit dsDNA HS Assay Kit (Thermo Fisher Scientific) to evaluate double-stranded DNA quantity). Each sample was normalized and pooled in equal molar concentrations. To confirm concentration pools underwent qPCR using KAPA Library Quantification Kit on QuantStudio 6 Flex before sequencing. Pools were sequenced on an Illumina NovaSeq6000 sequencer with the following parameters: 28 bp, read 1; 8 bp, 7 index; and 91 bp, read 2.

Raw RNA reads were processed with cellranger using mm10 from 10× as the reference genome to create filtered gene expression matrixes. Cell barcodes detected by cellranger were used as input to CITESeq for hashtagged sequence data (solid organs) generating a counts matrix with cell barcodes and hashtag oligo sequences per cell. The HTODemux function from Seurat was then used to identify clusters and classify cells according to their barcodes, including negative and doublet cells. Quality control metrics were generated using Scater followed by single-cell object conversion to Seurat objects, merging of objects and then analyses run using the standard Seurat pipeline (Rahrmann et al 2022—Supplementary Tables 26, 28 and 30).

SCS profiles of human CTCs (GSE75367; GSE74639; GSE60407; GSE67980; GSE114704; GSE144494) and 500 cells from Illumina 10× for human PBMC raw counts were merged in python v.3.7.3 using the pandas library. Only common genes between datasets were analyzed. Seurat objects were created from PBMCs and CTCs. Following this step, data were analyzed using the standard Seurat pipeline (Rahrmann et al 2022—Supplementary Table 33).

For direct comparison of human CTCs and mouse tCZCs, 15,328 orthologs were identified and profiles processed through the standard Seurat workflow that includes a per-cell normalization of each gene expression count. Enrichment of a hemoglobin gene expression was carried out in UCell and enrichment scores generated with a two-tailed Mann-Whitney U statistic.

### REFERENCES FOR EXAMPLES 6 TO 10

1. Dillekås, H., Rogers, M. S. & Straume, O. Are 90% of deaths from cancer caused by metastases? Cancer Med. 8, 5574-5576 (2019).
2. Ganesh, K. & Massagué, J. Targeting metastatic cancer. Nat. Med. 27, 34-44 (2021).
3. Massagué, J. & Obenauf, A. C. Metastatic colonization by circulating tumour cells. Nature 529, 298-306 (2016).
4. House, C. D. et al. Voltage-gated Na+ channel SCN5A is a key regulator of a gene transcriptional network that controls colon cancer invasion. Cancer Res. 70, 6957-6967 (2010).
5. Sheth, M. & Esfandiari, L. Bioelectric dysregulation in cancer initiation, promotion, and progression. Front. Oncol. 12, 846917 (2022).
6. Wang, T. et al. The calcium pump PMCA4 prevents epithelial-mesenchymal transition by inhibiting NFATc1-ZEB1 pathway in gastric cancer. Biochim. Biophys. Acta Mol. Cell. Res. 1867, 118833 (2020).
7. Nguyen, B. et al. Genomic characterization of metastatic patterns from prospective clinical sequencing of 25,000 patients. Cell 185, 563-575.e11 (2022).
8. Ganesh, K. et al. L1 CAM defines the regenerative origin of metastasis-initiating cells in colorectal cancer. Nat. Cancer 1, 28-45 (2020).
9. Laughney, A. M. et al. Regenerative lineages and immune-mediated pruning in lung cancer metastasis. Nat. Med. 26, 259-269 (2020).
10. Pastushenko, I. et al. Identification of the tumour transition states occurring during EMT. Nature 556, 463-468 (2018).
11. Chua, H. C., Wulf, M., Weidling, C., Rasmussen, L. P. & Pless, S. A. The NALCN channel complex is voltage sensitive and directly modulated by extracellular calcium. Sci. Adv. 6, eaaz3154 (2020).
12. Kschonsak, M. et al. Structure of the human sodium leak channel NALCN. Nature 587, 313-318 (2020).
13. Lu, B. et al. The neuronal channel NALCN contributes resting sodium permeability and is required for normal respiratory rhythm. Cell 129, 371-383 (2007).
14. Bend, E. G. et al. NALCN channelopathies: distinguishing gain-of-function and loss-of-function mutations. Neurology 87, 1131-1139 (2016).
15. Zhu, L. et al. Multi-organ mapping of cancer risk. Cell 166, 1132-1146 (2016).
16. Ren, D. Sodium leak channels in neuronal excitability and rhythmic behaviors. Neuron 72, 899-911 (2011).
17. Cochet-Bissuel, M., Lory, P. & Monteil, A. The sodium leak channel, NALCN, in health and disease. Front. Cell. Neurosci. 8, 132 (2014).
18. Martincorena, I. et al. Universal patterns of selection in cancer and somatic tissues. Cell 171, 1029-1041 (2017).
19. Weinstein, J. N. et al. The Cancer Genome Atlas Pan-Cancer analysis project. Nat. Genet. 45, 1113-1120 (2013).
20. Joerger, A. C. & Fersht, A. R. The p53 pathway: origins, inactivation in cancer, and emerging therapeutic approaches. Annu. Rev. Biochem. 85, 375-404 (2016).
21. Smart, O. S., Neduvelil, J. G., Wang, X., Wallace, B. A. & Sansom, M. S. P. OLE: a program for the analysis of the pore dimensions of ion channel structural models. J. Mol. Graph. 14, 354-360 (1996).
22. Newport, T. D., Sansom, M. S. P. & Stansfeld, P. J. The MemProtMD database: a resource for membrane-embedded protein structures and their lipid interactions. Nucleic Acids Res. 47, D390-D397 (2019).
23. Stansfeld, P. J. et al. MemProtMD: automated insertion of membrane protein structures into explicit lipid membranes. Structure 23, 1350-1361 (2015).
24. Koplev, S., Lin, K., Dohlman, A. B. & Ma'ayan, A. Integration of pan-cancer transcriptomics with RPPA proteomics reveals mechanisms of epithelial-mesenchymal transition. PLoS Comput. Biol. 14, e1005911 (2018).
25. El Marjou, F. et al. Tissue-specific and inducible Cre-mediated recombination in the gut epithelium. Genesis 39, 186-193 (2004).
26. Jackstadt, R. et al. Epithelial NOTCH signaling rewires the tumor microenvironment of colorectal cancer to drive poor-prognosis subtypes and metastasis. Cancer Cell 36, 319-336 (2019).
27. Magnuson, M. A. & Osipovich, A. B. Pancreas-specific Cre driver lines and considerations for their prudent use. Cell Metab. 18, 9-20 (2013).
28. Hingorani, S. R. et al. Preinvasive and invasive ductal pancreatic cancer and its early detection in the mouse. Cancer Cell 4, 437-450 (2003).
29. Zhu, L. et al. Prominin 1 marks intestinal stem cells that are susceptible to neoplastic transformation. Nature 457, 603-607 (2009).
30. Pantel, K. & Alix-Panabieres, C. Liquid biopsy and minimal residual disease—latest advances and implications for cure. Nat. Rev. Clin. Oncol. 16, 409-424 (2019).
31. Miyamoto, D. T. et al. RNA-Seq of single prostate CTCs implicates noncanonical Wnt signaling in antiandrogen resistance. Science 349, 1351-1356 (2015).
32. Ting, D. T. et al. Single-cell RNA sequencing identifies extracellular matrix gene expression by pancreatic circulating tumor cells. Cell Rep. 8, 1905-1918 (2014).
33. Zheng, Y. et al. Expression of p-globin by cancer cells promotes cell survival during blood-borne dissemination. Nat. Commun. 8, 14344 (2017).
34. Ebright, R. Y. et al. Deregulation of ribosomal protein expression and translation promotes breast cancer metastasis. Science 367, 1468-1473 (2020).
35. Jordan, N. V. et al. HER2 expression identifies dynamic functional states within circulating breast cancer cells. Nature 537, 102-106 (2016).
36. Dimitrov-Markov, S. et al. Discovery of new targets to control metastasis in pancreatic cancer by single-cell transcriptomics analysis of circulating tumor cells. Mol. Cancer Ther. 19, 1751-1760 (2020).
37. Todd, D. J. & Kay, J. Gadolinium-induced fibrosis. Annu. Rev. Med. 67, 273-291 (2016).
38. Wagner, B. et al. Nephrogenic systemic fibrosis: evidence for oxidative stress and bone marrow-derived fibrocytes in skin, liver, and heart lesions using a 5/6 nephrectomy rodent model. Am. J. Pathol. 181, 1941-1952 (2012).
39. Habib, A. R. et al. A systematic review of the clinical efficacy and safety of CFTR ModulatORS IN CYstic fibrosis. Sci. Rep. 9, 7234 (2019).
40. Pantel, K., Brakenhoff, R. H. & Brandt, B. Detection, clinical relevance and specific biological properties of disseminating tumour cells. Nat. Rev. Cancer 8, 329-340 (2008).
41. Kolling, S. et al. ‘Metastatic cancer of unknown primary’ or ‘primary metastatic cancer’?Front. Oncol. 9, 1546 (2020).
42. Martincorena, I. et al. Tumor evolution. High burden and pervasive positive selection of somatic mutations in normal human skin. Science 348, 880-886 (2015).
43. Martincorena, I. et al. Somatic mutant clones colonize the human esophagus with age. Science 362, 911-917 (2018).
44. Klein, C. A. Parallel progression of primary tumours and metastases. Nat. Rev. Cancer 9, 302-312 (2009).
45. Barbado, M., Fablet, K., Ronjat, M. & De Waard, M. Gene regulation by voltage dependent calcium channels. Biochim. Biophys. Acta 1793, 1096-1104 (2009).
46. Barker, N. et al. Lgr5(+ve) stem cells drive self-renewal in the stomach and build long-lived gastric units in vitro. Cell Stem Cell 6, 25-36 (2010).
47. Robinson, G. et al. Novel mutations target distinct subgroups of medulloblastoma. Nature 488, 43-48 (2012).
48. Lu, B. et al. Peptide neurotransmitters activate a cation channel complex of NALCN and UNC-80. Nature 457, 741-744 (2009).
49. Tronche, F. et al. Disruption of the glucocorticoid receptor gene in the nervous system results in reduced anxiety. Nat. Genet. 23, 99-103 (1999).
50. Ventura, A. et al. Restoration of p53 function leads to tumour regression in vivo. Nature 445, 661-665 (2007).
51. Madisen, L. et al. A robust and high-throughput Cre reporting and characterization system for the whole mouse brain. Nat. Neurosci. 13, 133-140 (2010).
52. Jackson, E. L. et al. Analysis of lung tumor initiation and progression using conditional expression of oncogenic K-ras. Genes Dev. 15, 3243-3248 (2001).
53. Marino, S., Vooijs, M., van Der Gulden, H., Jonkers, J. & Berns, A. Induction of medulloblastomas in p53-null mutant mice by somatic inactivation of Rb in the external granular layer cells of the cerebellum. Genes Dev. 14, 994-1004 (2000).
54. Patmore, D. M. et al. DDX3X suppresses the susceptibility of hindbrain lineages to medulloblastoma. Dev. Cell 54, 455-470.e5 (2020).
55. Gao, J. et al. Integrative analysis of complex cancer genomics and clinical profiles using the cBioPortal. Sci. Signal. 6, pl1 (2013).
56. Goldman, M. et al. Visualizing and interpreting cancer genomics data via the Xena platform. Nat. Biotechnol. 38, 675-678 (2020).
57. Monticelli, L. et al. The MARTINI coarse-grained force field: field: extension to proteins. J. Chem. Theory Comput. 4, 819-834 (2008).

1. A method for the detection or prognosis of cancer and/or metastasis comprising:
analysing a tumour sample obtained from a subject,
determining the presence of at least one mutation within sodium leak channel (NALCN) in the tumour sample, compared to a reference sample,
determining whether the at least one mutation causes a reduction in the pore size of NALCN, and
where the mutation causes a reduction in the pore size of NALCN, using the reduction in pore size to determine a risk score of cancer and/or metastasis.

2. The method according to claim 1, wherein the reference sample is a sample of germline DNA obtained from said subject, or a sample of germline DNA obtained from a healthy subject.

3. The method of claim 1 or 2, wherein computational modelling is used to determine whether the at least one mutation causes a reduction in pore size of NALCN.

4. The method according to claim 3, wherein computational modelling is performed using HOLE, CHAP, CAVER, or MOLE.

5. The method according to claim 3 or claim 4, wherein the reduction in NALCN pore size is calculated by determining the difference in size of the ion-selectivity filter radius in the NALCN variant comprising a mutation compared to the wild-type NALCN filter radius.

6. The method according to claim 3 or claim 4, wherein the reduction in NALCN pore size is calculated by determining the difference in size of the gate radius in the NALCN variant comprising a mutation compared to the wild-type NALCN gate radius.

7. A method for the detection or prognosis of cancer and/or metastasis comprising:
analysing a biological sample obtained from a subject, to assess the activity of sodium leak channel (NALCN),
providing a risk score of cancer and/or metastasis based on the level of activity of NALCN.

8. The method according to claim 7, wherein the activity of NALCN is assessed by whole-cell electrophysiology, a fluorescence assay, a membrane potential sensing dye, and/or an ion flux assay.

9. The method according to claim 7, further comprising a step of comparing the level of activity of NALCN in the biological sample with a reference value.

10. A method for the detection or prognosis of cancer and/or metastasis comprising:
analysing a biological sample to detect the presence of one or more mutations which correspond to a reduction of function of NALCN, and
providing a risk score of cancer and/or metastasis based on the presence of one or more mutations which correspond to a reduction of function of NALCN.

11. The method according to any one of claims 1 to 6 or claim 10, wherein the one or more mutations are located in the pore turret domain or voltage sensing domain of NALCN.

12. The method according to any one of claims 1 to 6 or claims 10 to 11, wherein the one or more mutations are selected from the mutations identified in Table 2.

13. The method according to any one of claims 10 to 12, wherein computational modelling is used to determine whether the one or more mutations which correspond to a reduction of function of NALCN causes a reduction in pore size of NALCN.

14. The method according to claim 13, wherein computational modelling is performed using HOLE, CHAP, CAVER, or MOLE.

15. The method according to claim 13 or claim 14, wherein the reduction in NALCN pore size is calculated by determining the difference in size of the ion-selectivity filter radius in the NALCN variant comprising a mutation compared to the wild-type NALCN filter radius.

16. The method according to claim 13 or claim 14, wherein the reduction in NALCN pore size is calculated by determining the difference in size of the gate radius in the NALCN variant comprising a mutation compared to the wild-type NALCN gate radius.

17. The method according to any preceding claim, wherein the method comprises a further step of identifying the stage of the cancer based on the one or more mutations that are identified.

18. The method according to any preceding claim, wherein the method comprises a further step of selecting a treatment.

19. The method according to any preceding claim, wherein the cancer is selected from gastric cancer, gastric adenocarcinoma, colorectal cancer, lung cancer, non-small cell lung cancer, lung adenocarcinoma, lung squamous cell carcinoma, bone cancer, pancreatic cancer, colon cancer, colorectal cancer, skin cancer, cancer of the head or neck, head and neck squamous cell carcinoma, melanoma, uterine cancer, ovarian cancer, rectal cancer, cancer of the anal region, stomach cancer, testicular cancer, breast cancer, brain cancer, hepatocellular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, kidney cancer, sarcoma of soft tissue, cancer of the urethra, cancer of the bladder, renal cancer, thymoma, urothelial carcinoma leukemia, prostate cancer, prostatic adenocarcinoma mesothelioma, adrenocortical carcinoma, lymphomas, such as such as Hodgkin's disease, non-Hodgkin's, and multiple myelomas.

20. A method for determining the activity of NALCN comprising:
analysing a biological sample to detect one or more mutations identified in Table 2, wherein the presence of one or more mutation identified in Table 2 indicates reduced activity of NALCN.

21. The method according to any of claims 1 to 6 or 10 to 20, wherein the mutations are detected via allele-specific polymerase chain reaction (PCR), high resolution melting curve analysis, genomic sequencing fluorescence in situ hybridization (FISH); comparative genomic hybridization (CGH), Restriction fragment length polymorphism RELP), amplification refractory mutation system (ARMS), reverse transcriptase PCR (RT-PCR), real-time PCR, multiplex ligation-dependent probe amplification (MLPA), denaturing gradient gel electrophoresis (DGGE), single strand conformational polymorphism (SSCP), chemical cleavage of mismatch (CCM), protein truncation test (PTT), or oligonucleotide ligation assay (OLA).

22. The method according to any preceding claim wherein the biological sample is analysed in vitro or ex vivo.

23. The method according to any preceding claim, wherein the biological sample is a tissue sample or a tumour sample.

24. A kit comprising reagents for the detection of one or more mutations in NALCN, wherein the mutation correlates to a reduction in activity of NALCN and/or a reduction in pore size of NALCN and optionally instructions for use.

25. A composition comprising reagents for the detection of one or more mutations in NALCN, wherein the mutation correlates to a reduction in activity of NALCN and/or a reduction in pore size of NALCN.

26. The kit according to claim 24 or composition according to claim 25, wherein the mutation is selected from one or more of the mutations listed in Table 2.

27. The kit according to claim 24 or composition according to claim 25, wherein the reagents are suitable for carrying out allele-specific polymerase chain reaction (PCR), high resolution melting curve analysis, genomic sequencing fluorescence in situ hybridization (FISH); comparative genomic hybridization (CGH), Restriction fragment length polymorphism RELP), amplification refractory mutation system (ARMS), reverse transcriptase PCR (RT-PCR), real-time PCR, multiplex ligation-dependent probe amplification (MLPA), denaturing gradient gel electrophoresis (DGGE), single strand conformational polymorphism (SSCP), chemical cleavage of mismatch (CCM), protein truncation test (PTT), or oligonucleotide ligation assay (OLA).

28. A computer-implemented method for determine a risk score of cancer and/or metastasis, the method comprising:
obtaining data indicating presence of at least one mutation in a sodium leak channel, NALCN, in a tumour sample;
inputting the data into a computational model of NALCN that simulates effects of mutations on NALCN;
determining, using the computational model, whether the at least one mutation causes a reduction in a pore size of NALCN; and
outputting, when the at least one mutation is determined to cause a reduction in pore size of NALCN, a risk score of cancer and/or metastasis.


SEQUENCE LISTING (30 sequences)

SEQ ID NO: 1 - protein, 1738 aa, Homo sapiens
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRFGGRTTMKSVVCKMNPMTDAASCGSEVKKWWTRQLTVESDESGDDLLDI

SEQ ID NO: 2 - protein, 173 aa, Homo sapiens
INKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTY

SEQ ID NO: 3 - protein, 57 aa, Homo sapiens
HCVVNDTKPGNVTWNSLAIPDTHCSPELEEGYQCPPGFKCMDLEDLGLSRQELGYSG

SEQ ID NO: 4 - protein, 74 aa, Homo sapiens
FNEIGTSIFTVYEAASQEGWVFLMYRAIDSFPRWRSYFYFITLIFFLAWLVKNVFIAVIIETFAEIRVQFQQMW

SEQ ID NO: 5 - skipped

SEQ ID NO: 6 - protein, 115 aa, Homo sapiens
ACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKIS

SEQ ID NO: 7 - protein, 11 aa, Homo sapiens
PALEDFVYKIF

SEQ ID NO: 8 - protein, 105 aa, Homo sapiens
GPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELD

SEQ ID NO: 9 - skipped

SEQ ID NO: 10 - protein, 10 aa, Homo sapiens
EDLKKLKQLK

SEQ ID NO: 11 - protein, 158 aa, Homo sapiens
EHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLV

SEQ ID NO: 12 - protein, 12 aa, Homo sapiens
PQMRKVVRELFS

SEQ ID NO: 13 - protein, 58 aa, Homo sapiens
KLAKCNDPNIIRREDCNGIFRINVSVSKNLNLKLRPGEKKPGFWVPRVWANPRNFNFD

SEQ ID NO: 14 - protein, 64 aa, Homo sapiens
NVGNAMLALFEVLSLKGWVEVRDVIIHRVGPIHGIYIHVFVFLGCMIGLTLFVGVVIANFNENK

SEQ ID NO: 15 - skipped

SEQ ID NO: 16 - protein, 33 aa, Homo sapiens
GTALLTVDQRRWEDLKSRLKIAQPLHLPPRPDN

SEQ ID NO: 17 - protein, 123 aa, Homo sapiens
DGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHV

SEQ ID NO: 18 - protein, 15 aa, Homo sapiens
TLKMLLLTVVVSMYK

SEQ ID NO: 19 - protein, 19 aa, Homo sapiens
FGTVKYGENINRHANFSSA

SEQ ID NO: 20 - protein, 23 aa, Homo sapiens
KAITVLFRIVTGEDWNKIMHDCM

SEQ ID NO: 21 - skipped

SEQ ID NO: 22 - protein, 20 aa, Homo sapiens
VQPPFCTPDEFTYWATDCGN

SEQ ID NO: 23 - protein, 142 aa, Homo sapiens
LLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQ

SEQ ID NO: 24 - DNA, 17 bp, synthetic construct
  source 1..17: mol_type = other DNA
gccctcagcccccaaac

SEQ ID NO: 25 - DNA, 20 bp, synthetic construct
  source 1..20: mol_type = other DNA
ggaagctgtgtctggcatgg

SEQ ID NO: 26 - DNA, 21 bp, synthetic construct
  source 1..21: mol_type = other DNA
aggtcggtgtgaacggatttg

SEQ ID NO: 27 - DNA, 23 bp, synthetic construct
  source 1..23: mol_type = other DNA
tgtagaccatgtagttgaggtca

SEQ ID NO: 28 - DNA, 25 bp, synthetic construct
  source 1..25: mol_type = other DNA
attgtccgtgagattgctcatcacc

SEQ ID NO: 29 - DNA, 25 bp, synthetic construct
  source 1..25: mol_type = other DNA
gcaccagctatatgtccctctcacg

SEQ ID NO: 30 - DNA, 27 bp, synthetic construct
  source 1..27: mol_type = other DNA
ggaaaatgaccacttcctagcagaagc